(19) United States
(12) Patent Application Publication
(10) Pub. No.: US 2026/0337193 A1
(43) Pub. Date: Oct. 29, 2026

(54) SMALL MOLECULE INHIBITORS OF UBIQUITIN SPECIFIC PROTEASE 1 (USP1) AND USES THEREOF

(71) Applicant: Insilico Medicine IP Limited, Central, Hong Kong (CN)

(72) Inventors: Jianping Wu, Pudong New Area, Shanghai (CN); Jinxin Liu, Pudong New Area, Shanghai (CN); Luoheng Qin, Pudong New Area, Shanghai (CN)

(21) Appl. No.: 19/485,062

(22) PCT Filed: May 16, 2024
(86) PCT No.: PCT/IB2024/054769

§ 371 Date: Nov. 17, 2025

(30) Foreign Application Priority Data
May 18, 2023 (WO) PCT/CN2023/095070

(51) Int. Cl. C07D 471/04; A61K 31/427; A61K 31/444; A61K 31/497; A61K 31/506; A61K 31/519; A61P 35/00; C07D 401/14; C07D 487/04

(52) U.S. Cl. CPC C07D 471/04; A61K 31/427; A61K 31/444; A61K 31/497; A61K 31/506; A61K 31/519; A61P 35/00; C07D 401/14; C07D 487/04

(57) ABSTRACT

The disclosure provides for small molecules inhibitory compounds of ubiquitin specific protease 1 (USP1) and compositions comprising the same. The disclosure further provides methods for targeting ubiquitin specific protease 1 (USP1) and methods of treating diseases or disorders related to USP1, such as cancer.

### CROSS-REFERENCE TO RELATED APPLICATION

This application claims priority to PCT patent application no. PCT/CN2023/095070, filed May 18, 2023, the entire contents of which are incorporated herein by reference.

### BACKGROUND

Ubiquitin specific protease 1 (USP1) is a gene that plays a role in a DNA damage repair. Compounds and pharmaceutical compositions targeting USP1, and methods of treatment for USP1-related diseases and disorders, like certain cancers, have not been widely developed. Therefore, there remains a need to address methods of treating USP1-related diseases.

### SUMMARY

The present disclosure addresses the above need and provides additional advantages as well.

In one aspect, described herein is a compound having the structure of Formula (I), or a salt thereof,

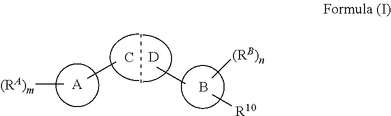

wherein,
ring A is aryl or heteroaryl;
ring B is phenyl, phenyl isostere, or heteroaryl;
ring C is an optionally substituted 5 to 6 membered heteroaryl;
ring D is an optionally substituted 5 to 6 membered heterocycloalkyl or an optionally substituted 5 to 6 membered cycloalkyl;
each of R^{A} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{A1};
or two R^{A} are taken together with the intervening atoms to which they are attached to form a cycloalkyl, heterocycloalkyl, aryl, or heteroaryl, wherein each of which independently optionally substituted with one or more R^{A1};
each R^{A1} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl; wherein each of the alkyl, heteroalkyl, alkenyl, alkynyl, cycloalkyl, heterocycloalkyl, aryl, and heteroaryl is independently optionally substituted with one or more R;
m is 0, 1, 2, 3, 4, or 5;
each of R^{B} is independently selected from halogen, —CN, —NO₂, —OH, —OR^{a}, —OC(═O)R^{a}, —OC(═O)OR, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{B1};
n is 0, 1, 2, 3, or 4;
each R^{B1} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl; wherein each of the alkyl, heteroalkyl, alkenyl, alkynyl, cycloalkyl, heterocycloalkyl, aryl, and heteroaryl is independently optionally substituted with one or more R;
R¹⁰ is cycloalkyl, heterocycloalkyl, aryl, or heteroaryl, each of which is independently optionally substituted with one or more R^{B1};
each R^{a} is independently C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and each R is independently halogen, —CN, —OH, —SF₅, —SH, —S(═O)C₁-C₃ alkyl, —S(═O)₂C₁-C₃ alkyl, —S(═O)₂NH₂, —S(═O)₂NHC₁-C₃ alkyl, —S(═O)₂N(C₁-C₃ alkyl)₂, —S(═O)(═NC₁-C₃ alkyl)(C₁-C₃ alkyl), —NH₂, —NHC₁-C₃ alkyl, —N(C₁-C₃ alkyl)₂, —N═S(═O)(C₁-C₃ alkyl)₂, —C(═O)C₁-C₃ alkyl, —C(═O)OH, —C(═O)OC₁-C₃ alkyl, —C(═O)NH₂, —C(═O)NHC₁-C₃ alkyl, —C(═O)N(C₁-C₃ alkyl)₂, —P(═O)(C₁-C₃ alkyl)₂, C₁-C₃ alkyl, C₁-C₃alkoxy, C₁-C₃ haloalkyl, C₁-C₃ haloalkoxy, C₁-C₃ hydroxyalkyl, C₁-C₃ aminoalkyl, C₁-C₃ heteroalkyl, or C₃-C₆cycloalkyl;
or two R on the same atom form an oxo.

In some embodiments, the compound has a structure of Formula (IIA), or a pharmaceutically acceptable salt thereof,

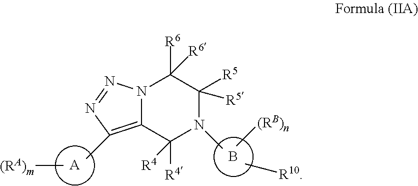

In some embodiments, the compound has a structure of Formula (IIB), or a pharmaceutically acceptable salt thereof,

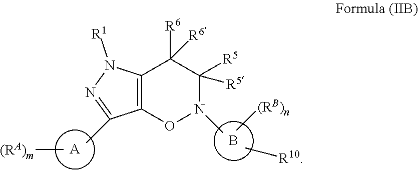

In one aspect, described herein is a compound having the structure of Formula (IIC), or a pharmaceutically acceptable salt thereof,

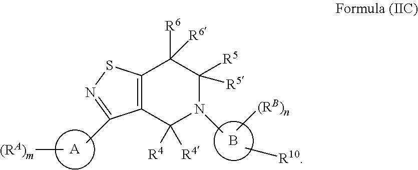

In one aspect, described herein is a compound having the structure of Formula (IID), or a pharmaceutically acceptable salt thereof,

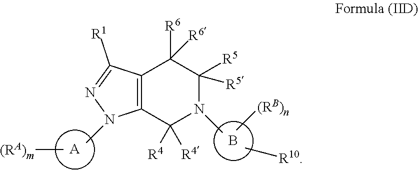

In one aspect, described herein is a compound having the structure of Formula (IID′), or a pharmaceutically acceptable salt thereof,

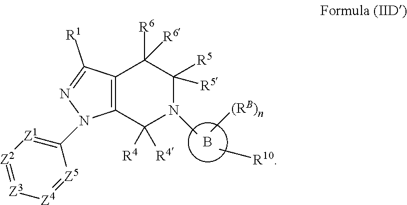

In one aspect, described herein is a compound having the structure of Formula (IID″), or a pharmaceutically acceptable salt thereof,

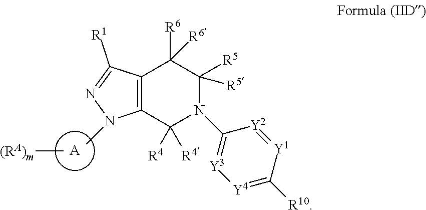

In one aspect, described herein is a compound having the structure of Formula (IIE), or a pharmaceutically acceptable salt thereof,

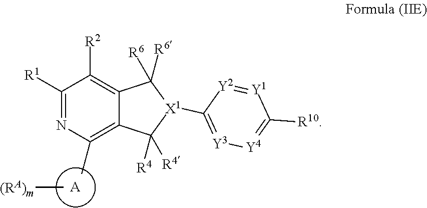

In one aspect, described herein is a compound having the structure of Formula (IIF), or a pharmaceutically acceptable salt thereof,

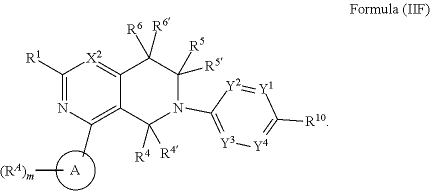

In one aspect, described herein is a compound having the structure of Formula (IIG), or a pharmaceutically acceptable salt thereof,

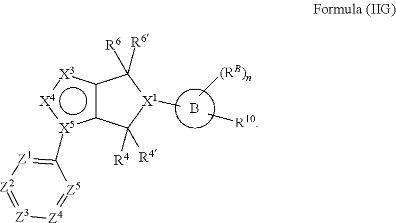

In one aspect, described herein is a pharmaceutical composition comprising a compound described herein or a pharmaceutically acceptable salt thereof, and a pharmaceutically acceptable carrier or excipient.

In one aspect, described herein is a method of modulating ubiquitin specific protease 1 (USP1) in a subject, the method comprising administering to a subject a compound described herein, or a pharmaceutically acceptable salt thereof, or a pharmaceutical composition of a compound described herein.

In one aspect, described herein is a method of inhibiting ubiquitin specific protease 1 (USP1) in a subject, the method comprising administering to the subject a compound described herein, or a pharmaceutically acceptable salt thereof, or a pharmaceutical composition of a compound described herein.

In one aspect, described herein is a method of inhibiting or reducing DNA repair activity modulated by ubiquitin specific protease 1 (USP1) in a subject, the method comprising administering to the subject in need thereof an effective amount of a compound described herein, or a pharmaceutically acceptable salt thereof, or a pharmaceutical composition of a compound described herein.

In one aspect, described herein is a method of treating a disease or disorder associated with ubiquitin specific protease 1 (USP1) in a subject, the method comprising administering to the subject a compound described herein, or a pharmaceutically acceptable salt thereof, or a pharmaceutical composition of a compound described herein.

In one aspect, described herein is a method of treating a disease or disorder associated with modulation of ubiquitin specific protease 1 (USP1) in a subject, the method comprising administering to the subject a compound described herein, or a pharmaceutically acceptable salt thereof, or a pharmaceutical composition of a compound described herein. In some embodiments, the disease or disorder is cancer.

In one aspect, described herein is a method of treating cancer in a subject, the method comprising administering to the subject in need thereof an effective amount of a compound described herein, or a pharmaceutically acceptable salt thereof, or a pharmaceutical composition of a compound described herein. In some embodiments, the cancer is selected from the group consisting of lung cancer, non-small cell lung cancer (NSCLC), colon cancer, bladder cancer, osteosarcoma, ovarian cancer, skin cancer, and breast cancer. In some embodiments, the cancer is ovarian cancer. In some embodiments, the cancer is a breast cancer. In some embodiments, the cancer is ovarian cancer or breast cancer.

In some embodiments, the cancer comprises cancer cells with elevated levels of RAD 18. In some embodiments, the cancer is a DNA damage repair pathway deficient cancer. In some embodiments, the cancer is a PARP inhibitor resistant or refractory cancer. In some embodiments, the cancer is a BRCA1 mutant cancer and/or a BRCA2 mutant cancer. In some embodiments, the cancer is a BRAC1-deficient cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

While various embodiments of the disclosure have been shown and described herein, it will be obvious to those skilled in the art that such embodiments are provided by way of example only. Numerous variations, changes, and substitutions can occur to those skilled in the art without departing from the disclosure. It should be understood that various alternatives to the embodiments of the disclosure described herein can be employed.

### A. Definitions

Unless defined otherwise, all technical and scientific terms used herein have the same meaning as is commonly understood by one of skill in the art to which this disclosure belongs. All patents and publications referred to herein are incorporated by reference.

“Alkyl” refers to an optionally substituted straight-chain, or optionally substituted branched-chain saturated hydrocarbon mono-radical, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). Whenever it appears herein, a numerical range such as “C₁-C₃ alkyl” means that the alkyl group consists of 1 carbon atom, 2 carbon atoms, or 3 carbon atoms. In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). In other embodiments, examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. The alkyl is attached to the rest of the molecule by a single bond. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, —NO₂, or —C≡CH. In some embodiments, the alkyl is optionally substituted with oxo, halogen, —CN, —CF₃, —OH, or —OMe. In some embodiments, the alkyl is optionally substituted with halogen such as F.

As used herein, C₁-Cₓ(or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

“Alkoxy” refers to a radical bonded through an oxygen atom of the formula —O-alkyl, where alkyl is an alkyl chain as defined above. Unless stated otherwise specifically in the specification, an alkoxy group can be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Alkenyl” refers to an optionally substituted straight or branched hydrocarbon chain radical group containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The group can be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group can consist of 2 carbon atoms, 3 carbon atoms, 4 carbon atoms, 5 carbon atoms, or 6 carbon atoms. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight or branched hydrocarbon chain radical group containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). Whenever it appears herein, a numerical range such as “C₂-C₆ alkynyl” means that the alkynyl group can consist of 2 carbon atoms, 3 carbon atoms, 4 carbon atoms, 5 carbon atoms, or 6 carbon atoms. The alkynyl is attached to the rest of the molecule by a single bond, for example, ethynyl, propynyl, butynyl, pentynyl, hexynyl, 2-propynyl, 2-butynyl, 1,3-butadiynyl, and the like. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkylene” or “alkylene chain” refers to an optionally substituted straight or branched divalent hydrocarbon chain linking the rest of the molecule to a radical group containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene group can be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Aryl” refers to a radical derived from a hydrocarbon ring system comprising at least one aromatic ring. In some embodiments, an aryl comprises hydrogens and 6 to 30 carbon atoms.

The aryl radical can be a monocyclic, bicyclic, tricyclic, or tetracyclic ring system, which can include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, indane, indene, naphthalene, phenalene, phenanthrene, pleiadene, pyrene, and triphenylene. In some embodiments, the aryl is phenyl. Unless stated otherwise specifically in the specification, an aryl can be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆ alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂.

“Aralkyl” refers to a radical of the formula —R^{c}-aryl where R^{c} is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R is an alkynylene chain as defined above.

“Carbocycle” refers to a saturated, unsaturated or aromatic rings in which each atom of the ring is carbon. Carbocycle can include 3- to 10-membered monocyclic rings and 6- to 12-membered bicyclic rings (such as spiro, fused, or bridged rings). Each ring of a bicyclic carbocycle can be selected from saturated, unsaturated, and aromatic rings. An aromatic ring, e.g., phenyl, can be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. In an exemplary embodiment, an aromatic ring, e.g., phenyl, can be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle includes any combination of ring sizes such as 4-5 fused ring systems, 5-5 fused ring systems, 5-6 fused ring systems, 6-6 fused ring systems, 5-7 fused ring systems, 6-5 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, and naphthyl. The term “unsaturated carbocycle” refers to carbocycles with at least one degree of unsaturation and excluding aromatic carbocycles. Examples of unsaturated carbocycles include cyclohexadiene, cyclohexene, and cyclopentene. The term “saturated cycloalkyl” as used herein refers to a saturated carbocycle. Exemplary carbocycles include cyclopropyl, cyclopentyl, cyclohexyl, cyclohexenyl, adamantyl, phenyl, indanyl, norborane, and naphthyl. Carbocycles can be optionally substituted by one or more substituents such as those substituents described herein.

As used herein, “phenyl isostere” refers to a moiety or a functional group that exhibits similar physical, biological and/or chemical properties as a phenyl group. Exemplary phenyl isosteres include, without limitation, cubane, bicyclo[1.1.1]pentane (BCP), bicyclo[2.2.1]heptane, bicyclo[2.1.1]hexane, bicyclo[2.2.2]octane, adamantane, norbornene, closo-1,2-carborane, closo-1,7-carborane, closo-1,12-carborane, and ethynyl group. In some embodiments, the phenyl isostere is cubane. In some embodiments, the phenyl isostere is an ethynyl group.

“Cycloalkyl” refers to a stable, partially or fully saturated, monocyclic or polycyclic carbocyclic ring, which can include fused (when fused with an aryl or a heteroaryl ring, the cycloalkyl is bonded through a non-aromatic ring atom), bridged, or spiro ring systems.

Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihalomethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen can be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

The term “heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a stable 3- to 24-membered partially or fully saturated ring radical comprising 2 to 23 carbon atoms and at least one ring heteroatoms. In some embodiments, a heterocycloalkyl contains from one to 8 heteroatoms selected from the group consisting of nitrogen, oxygen, phosphorous, and sulfur. Unless stated otherwise specifically in the specification, the heterocycloalkyl radical can be a monocyclic, bicyclic, tricyclic, or tetracyclic ring system, which can include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical can be optionally oxidized; the nitrogen atom can be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heterocycle” or “heterocyclyl” refers to a saturated, unsaturated or aromatic ring comprising one or more ring heteroatoms. Exemplary heteroatoms include N, O, Si, P, B, and S atoms. Heterocycles include e.g., 3- to 10-membered monocyclic rings and 6- to 12-membered bicyclic rings (such as spiro, fused, or bridged rings). Unless stated otherwise specifically in the specification, the heterocyclyl radical is a monocyclic, bicyclic, tricyclic or tetracyclic ring system, which optionally includes fused, bridged, or spirocyclic ring systems. The heteroatoms in the heterocyclyl radical are optionally oxidized. One or more nitrogen atoms, if present, are optionally quaternized. The heterocyclyl radical can be partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted. For example, a heterocyclyl can be optionally substituted by one or more substituents by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—CN, —R^{b}—O—R^{e}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R is independently a direct bond or a straight or branched alkylene or alkenylene chain, and R is a straight or branched alkylene or alkenylene chain, and where each of the above substituents is unsubstituted unless otherwise indicated.

“Heteroaryl” or “aromatic heterocycle” refers to a ring system radical comprising carbon atom(s) and one or more ring heteroatoms (e.g., selected from the group consisting of nitrogen, oxygen, phosphorous, silicon, and sulfur), and at least one aromatic ring. In some embodiments, a heteroaryl is a 5- to 14-membered ring system radical comprising one to thirteen carbon atoms, one to six heteroatoms selected from the group consisting of nitrogen, oxygen, phosphorous, and sulfur. The heteroaryl radical can be a monocyclic, bicyclic, tricyclic, or tetracyclic ring system, which can include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical can be optionally oxidized; the nitrogen atom can be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen can have hydrogen substituents and/or any permissible substituents of organic compounds described herein which satisfy the valences of the heteroatoms.

In some embodiments, substituents can include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which can be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), SF⁵, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, can be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

As used in the specification and claims, the singular form “a”, “an” and “the” includes plural references unless the context clearly dictates otherwise.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder can refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, can include alleviating, abating or ameliorating a disease or condition symptoms, preventing additional symptoms, ameliorating the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group can be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 can comprise 1 to 10, 1 to 20, 1 to 30, and 1 to 40 in one direction, or 50 to 40, 50 to 30, 50 to 20, and 50 to 10 in the other direction.

### B. Compounds of the Disclosure

In one aspect, the disclosure provides a compound represented by Formula (I), or a pharmaceutically acceptable salt thereof:

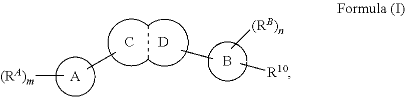

wherein,
ring A is aryl or heteroaryl;
ring B is phenyl, phenyl isostere, or heteroaryl;
ring C is an optionally substituted 5 to 6 membered heteroaryl;
ring D is an optionally substituted 5 to 6 membered heterocycloalkyl or an optionally substituted 5 to 6 membered cycloalkyl;
each of R^{A} is independently selected from halogen, —CN, —NO₂, —OH, —OR^{a}, —OC(═O)R^{a}, —OC(═O)OR, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{A1};
or two R^{A} are taken together with the intervening atoms to which they are attached to form a cycloalkyl, heterocycloalkyl, aryl, or heteroaryl, wherein each of which independently optionally substituted with one or more R^{A1};
each R^{A1} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl; wherein each of the alkyl, heteroalkyl, alkenyl, alkynyl, cycloalkyl, heterocycloalkyl, aryl, and heteroaryl is independently optionally substituted with one or more R;
m is 0, 1, 2, 3, 4, or 5;
each of R^{B} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{B1};
n is 0, 1, 2, 3, or 4;
each R^{B1} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl; wherein each of the alkyl, heteroalkyl, alkenyl, alkynyl, cycloalkyl, heterocycloalkyl, aryl, and heteroaryl is independently optionally substituted with one or more R;
R¹⁰ is cycloalkyl, heterocycloalkyl, aryl, or heteroaryl, each of which is independently optionally substituted with one or more R^{B1};
each R^{a} is independently C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃ alkyl, —S(═O)₂C₁-C₃ alkyl, —S(═O)₂NH₂, —S(═O)₂NHC₁-C₃ alkyl, —S(═O)₂N(C₁-C₃ alkyl)₂, —S(═O)(═NC₁—C₃ alkyl)(C₁-C₃ alkyl), —NH₂, —NHC₁-C₃ alkyl, —N(C₁-C₃ alkyl)₂, —N═S(═O)(C₁-C₃ alkyl)₂, —C(═O)C₁-C₃ alkyl, —C(═O)OH, —C(═O)OC₁—C₃ alkyl, —C(═O)NH₂, —C(═O)NHC₁—C₃ alkyl, —C(═O)N(C₁-C₃ alkyl)₂, —P(═O)(C₁-C₃ alkyl)₂, C₁-C₃ alkyl, C₁-C₃alkoxy, C₁-C₃ haloalkyl, C₁-C₃ haloalkoxy, C₁-C₃ hydroxyalkyl, C₁-C₃ aminoalkyl, C₁-C₃ heteroalkyl, or C₃-C₆cycloalkyl;
or two R on the same atom form an oxo,

In some embodiments of Formula (I),

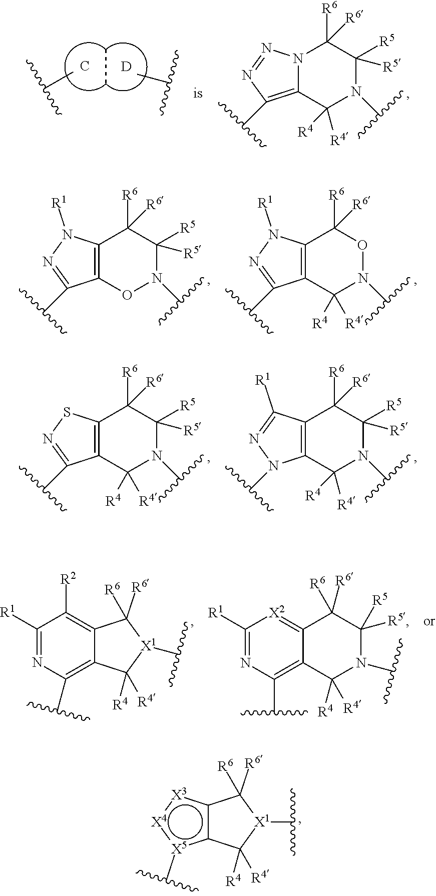

wherein
X¹ is N or CR^{X};
R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R;
X² is N or CR²;
X³ is N, NR^{1N}, CR¹, S or O;
X⁴ is N, NR^{2N}, CR², S or O;
X⁵ is N or C;
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

In some embodiments,

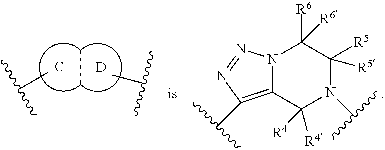

In some embodiments,

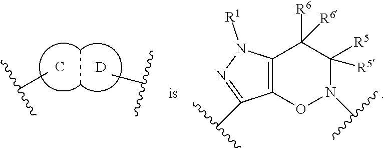

In some embodiments,

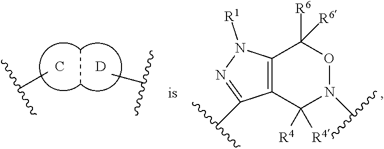

In some embodiments,

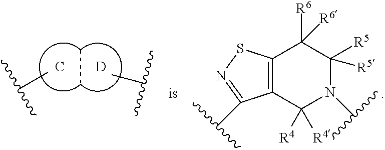

In some embodiments,

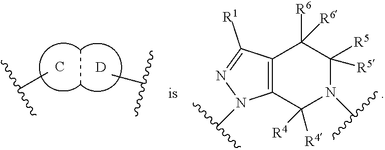

In some embodiments,

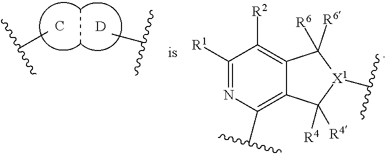

In some embodiments,

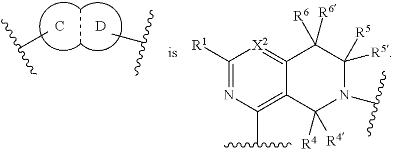

In some embodiments,

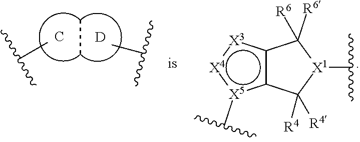

In some embodiments of a compound,

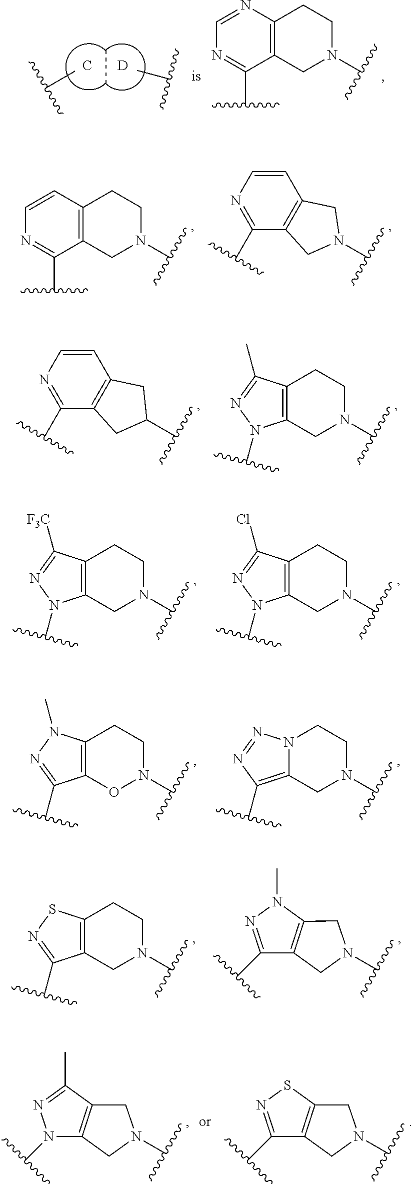

In some embodiments,

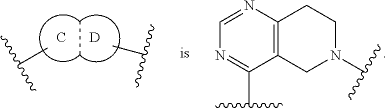

In some embodiments,

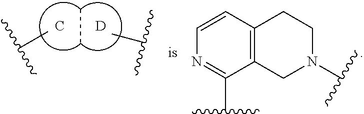

In some embodiments,

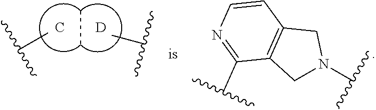

In some embodiments,

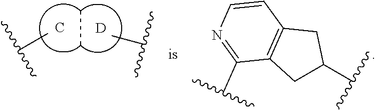

In some embodiments,

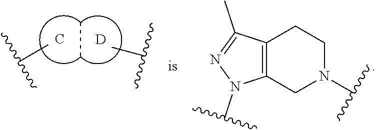

In some embodiments,

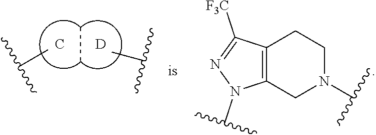

In some embodiments,

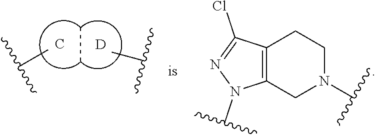

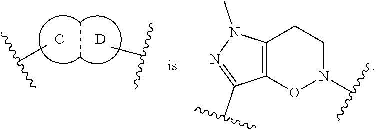

In some embodiments,

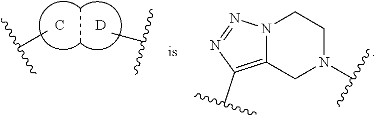

In some embodiments,

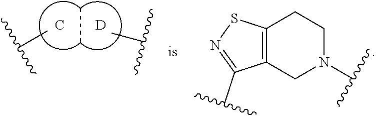

In some embodiments,

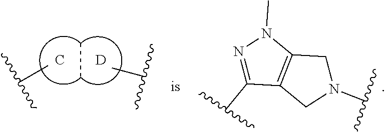

In some embodiments,

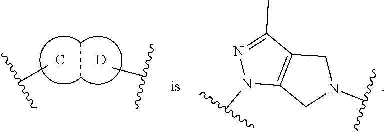

In some embodiments,

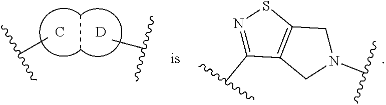

In some embodiments,

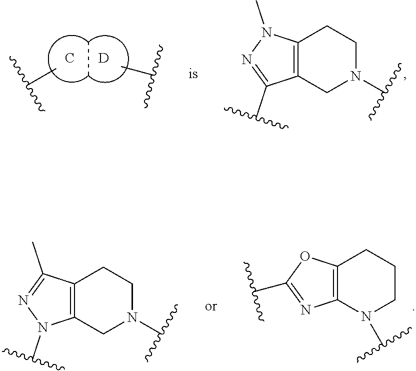

In some embodiments, ring C is a 5 membered heteroaryl, wherein the heteroaryl is optionally substituted. In some embodiments, ring C is optionally substituted 5 membered heteroaryl.

In some embodiments, ring C is 5 membered heteroaryl and ring D is 6 membered heteroaryl. In some embodiments, ring C is 5 membered heteroaryl and ring D is 6 membered heterocycloalkyl.

In some embodiments, each of ring C and ring D is independently optionally substituted with one or more substituents selected from halo, —CN, —OR^{a}, —SH, —SR^{a}, —NR^{c}R^{d}, optionally substituted C₁₋₆ alkyl, optionally substituted C₁₋₆ heteroalkyl, optionally substituted C₂₋₆ alkenyl, and optionally substituted C₂₋₆ alkynyl, and wherein the alkyl, heteroalkyl, alkenyl, or alkynyl is optionally substituted with one or more substituents independently selected from: halogen, amino, oxo, —OH, —NO₂, —CN, and C₁₋₃ alkoxyl.

In some embodiments, ring D is an aromatic, saturated or partially saturated 6 membered carbocycle or heterocycle, wherein each of the carbocycle or heterocycle is optionally substituted. In some embodiments, ring D is an optionally substituted aromatic 6 membered carbocycle. In some embodiments, ring D is an optionally substituted aromatic 6 membered heterocycle. In some embodiments, ring D is an optionally substituted saturated 6 membered carbocycle. In some embodiments, ring D is an optionally substituted saturated 6 membered heterocycle. In some embodiments, ring D is an optionally substituted partially saturated 6 membered carbocycle. In some embodiments, ring D is an optionally substituted partially saturated 6 membered heterocycle.

In some embodiments of a compound of Formula (I),

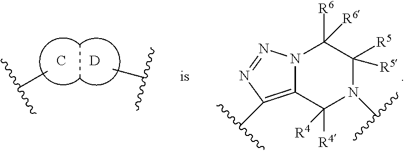

In one aspect, the disclosure provides a compound represented by Formula (IIA), or a pharmaceutically acceptable salt thereof:

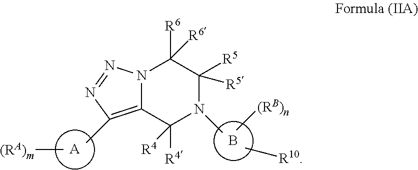

In some embodiments of a compound of Formula (I),

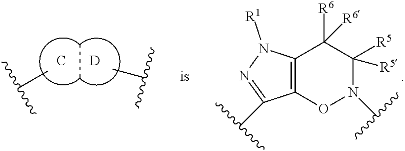

In one aspect, the disclosure provides a compound represented by Formula (IIB), or a pharmaceutically acceptable salt thereof:

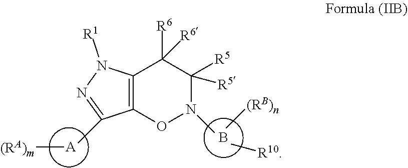

In some embodiments of a compound of Formula (I), is

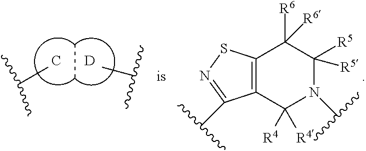

In one aspect, the disclosure provides a compound represented by Formula (IIC), or a pharmaceutically acceptable salt thereof:

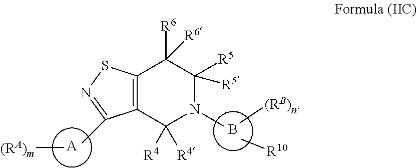

In some embodiments of a compound of Formula (I),

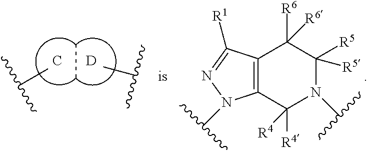

In one aspect, the disclosure provides a compound represented by Formula (IID), or a pharmaceutically acceptable salt thereof:

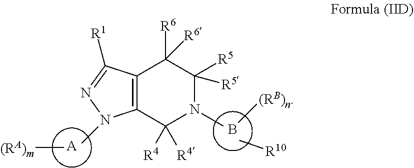

In one aspect, the disclosure provides a compound represented by Formula (IID′), or a pharmaceutically acceptable salt thereof:

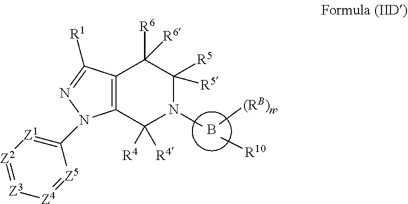

wherein,
Z¹ is N or CR^{Z1};
Z² is N or CR^{Z2};
Z³ is N or CR^{Z3};
Z⁴ is N or CR^{Z4}.
Z⁵ is N or CR^{Z4};
each of R^{Z1}, R^{Z2}, R^{Z3}, R^{Z4} and R^{Z5} is independently hydrogen or R^{A};
R¹ is halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

In some embodiments of a compound of Formula (IID′), each of R^{Z1}, R^{Z2}, R^{Z3}, R^{Z4} and R^{Z5} is independently hydrogen or R^{A}, provided that when Z¹ is CR^{Z1} and Z⁵ is CR^{Z5}, at least one of R^{Z1} and R^{Z5} is selected from R^{A}.

In one aspect, the disclosure provides a compound represented by Formula (IID″), or a pharmaceutically acceptable salt thereof:

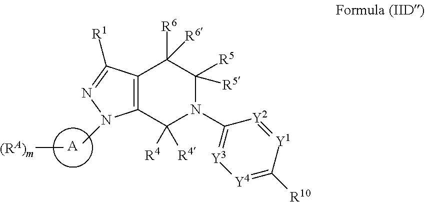

ring A is 5-membered heteroaryl;
Y¹ is N or CR^{Y1};
Y² is N or CR^{Y2};
Y³ is N or CR^{Y3};
Y⁴ is N or CR^{Y4};
each of R^{Y1}, R^{Y2}, R^{Y3} and R^{Y4} is independently hydrogen or R^{B};
R¹ is hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

In some embodiments of a compound of Formula (I),

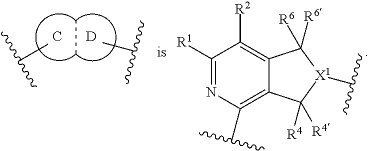

In one aspect, the disclosure provides a compound represented by Formula (IIE), or a pharmaceutically acceptable salt thereof:

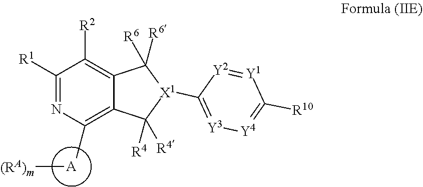

Y¹ is N or R^{Y1};
Y² is N or CR^{Y2};
Y³ is N or CR^{Y3};
Y⁴ is N or R^{Y4};
each of R^{Y1}, R^{Y2}, R^{Y3} and R^{Y4} is independently hydrogen or R^{B};
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
X¹ is N or CR^{X};
R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

In some embodiments of a compound of Formula (I),

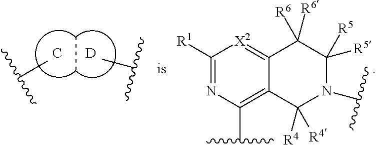

In one aspect, the disclosure provides a compound represented by Formula (IIF), or a pharmaceutically acceptable salt thereof:

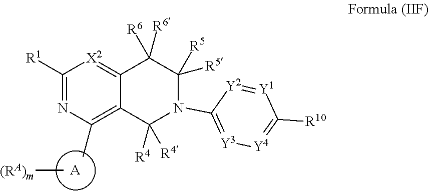

Y¹ is N or CR^{Y1};
Y² is N or CR^{Y2};
Y³ is N or CR^{Y3};
Y⁴ is N or CR^{Y4};
X² is N or CR²;
each of R^{Y1}, R^{Y2}, R^{Y3} and R^{Y4} is independently hydrogen or R^{B};
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

In some embodiments of a compound of Formula (I),

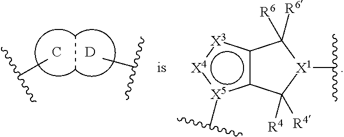

In one aspect, the disclosure provides a compound represented by Formula (IIG), or a pharmaceutically acceptable salt thereof:

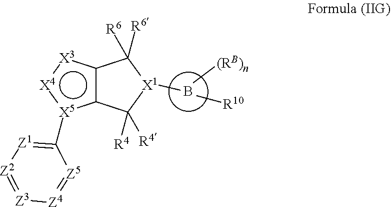

Z¹ is N or CR^{Z1};
Z² is N or CR^{Z2};
Z³ is N or CR^{Z3};
Z⁴ is N or CR^{Z4};
Z⁵ is N or CR^{Z4};
each of R^{Z1}, R^{Z2}, R^{Z3}, R^{Z4} and R^{Z5} is independently hydrogen or R^{A};
X³ is N, NR^{1N}, CR¹, S or O;
X⁴ is N, NR^{2N}, CR², S or O;
X⁵ is N or C;
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl;
X¹ is N or CR^{X};
R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

In some embodiments, each of R^{Z1}, R^{Z2}, R^{Z3}, R^{Z4} and R^{Z5} is independently hydrogen or R^{A}, provided that at least one of R^{Z1} and R^{Z5} is present and is selected from R^{A}.

In one aspect, the disclosure provides a compound represented by Formula (IIH), or a pharmaceutically acceptable salt thereof:

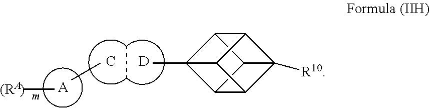

In some embodiments,

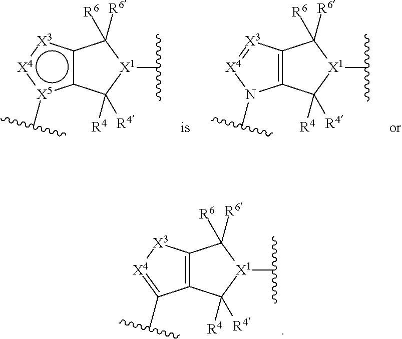

In some embodiments,

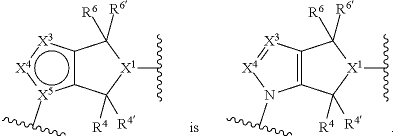

In some embodiments,

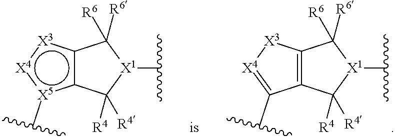

In some embodiments, X³ is N, NR^{1N}, CR¹, S, or O. In some embodiments, X³ is N, NR^{1N}, or CR¹. In some embodiments, X³ is N. In some embodiments, X³ is NR^{1N}. In some embodiments, X³ is CR¹. In some embodiments, X³ is S. In some embodiments, X³ is O.

In some embodiments, R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl. In some embodiments, R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R. In some embodiments, R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl. In some embodiments, R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl.

In some embodiments, RIN is hydrogen, C₁₋₆ alkyl, or C₁-C₆ haloalkyl, wherein the alkyl is optionally substituted with one or more R. In some embodiments, RIN is hydrogen, C₁₋₆ alkyl, or C₁-C₆ haloalkyl. In some embodiments, R^{1N} is hydrogen. In some embodiments, R^{1N} is C₁₋₆ alkyl. In some embodiments, R^{1N} is CH₃. In some embodiments, R^{1N} is C₁-C₆ haloalkyl.

In some embodiments, R^{2N} is hydrogen, C₁₋₆ alkyl, or C₁-C₆ haloalkyl, wherein the alkyl is optionally substituted with one or more R. In some embodiments, R^{2N} is hydrogen, C₁₋₆ alkyl, or C₁-C₆ haloalkyl. In some embodiments, R^{2N} is hydrogen. In some embodiments, R^{2N} is C₁₋₆ alkyl. In some embodiments, R^{2N} is CH₃. In some embodiments, R^{2N} is C₁-C₆ haloalkyl.

In some embodiments, R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R. In some embodiments, R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl.

In some embodiments, R¹ is halogen, C₁₋₆ alkyl, or C₁-C₆ haloalkyl, wherein the alkyl is optionally substituted with one or more R. In some embodiments, R¹ is halogen, C₁₋₆ alkyl, or C₁-C₆ haloalkyl. In some embodiments, R¹ is halogen. In some embodiments, R¹ is C₁₋₆ alkyl. In some embodiments, R¹ is CH₃. In some embodiments, R¹ is halogen. In some embodiments, R¹ is F. In some embodiments, R¹ is Cl. In some embodiments, R¹ is C₁-C₆ haloalkyl. In some embodiments, R¹ is CF₃.

In some embodiments, R² is halogen, C₁₋₆ alkyl, or C₁-C₆ haloalkyl, wherein the alkyl is optionally substituted with one or more R. In some embodiments, R² is halogen, C₁₋₆ alkyl, or C₁-C₆ haloalkyl. In some embodiments, R² is halogen. In some embodiments, R² is C₁₋₆ alkyl. In some embodiments, R² is CH₃. In some embodiments, R² is halogen. In some embodiments, R² is F. In some embodiments, R² is Cl. In some embodiments, R² is C₁-C₆ haloalkyl. In some embodiments, R² is CF₃.

In some embodiments, R¹ is halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R. In some embodiments, R¹ is halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂-6 alkynyl, cycloalkyl, or heterocycloalkyl.

In some embodiments, R² is halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R. In some embodiments, R² is halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl.

In some embodiments, X⁴ is N, NR^{2N}, CR², S, or O. In some embodiments, X⁴ is N, NR^{2N}, or CR². In some embodiments, X⁴ is N. In some embodiments, X⁴ is NR^{2N}. In some embodiments, X⁴ is CR². In some embodiments, X⁴ is S.

In some embodiments, X⁴ is O. In some embodiments, X⁵ is N or C. In some embodiments, X⁵ is N. In some embodiments, X⁵ is C.

In some embodiments, X¹ is N or CR^{X}. In some embodiments, X¹ is N. In some embodiments, X¹ is CR^{X}.

In some embodiments, R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R. In some embodiments, R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl. In some embodiments, R^{X} is hydrogen. In some embodiments, R^{X} is halogen. In some embodiments, R^{X} is C₁₋₆ alkyl. In some embodiments, R^{X} is C₁-C₆ haloalkyl. In some embodiments, R^{X} is C₁-C₆ hydroxyalkyl. In some embodiments, R^{X} is C₁-C₆ aminoalkyl. In some embodiments, R^{X} is C₁-C₆ heteroalkyl.

In some embodiments, Z¹ is N or CR^{Z1}. In some embodiments, Z¹ is N. In some embodiments, Z¹ is CR^{Z1}. In some embodiments, Z² is N or CR^{Z2}. In some embodiments, Z² is N. In some embodiments, Z² is CR^{Z2}. In some embodiments, Z³ is N or CR^{Z3}. In some embodiments, Z³ is N. In some embodiments, Z³ is CR^{Z3}. In some embodiments, Z⁴ is N or CR^{Z4}. In some embodiments, Z⁴ is N. In some embodiments, Z⁴ is CR^{Z4}. In some embodiments, Z⁵ is N or CR^{Z5}. In some embodiments, Z⁵ is N. In some embodiments, Z⁵ is CR^{Z5}.

In some embodiments, each of R^{Z1}, R^{Z2}, R^{Z3}, R^{Z4} and R^{Z5} is independently hydrogen or R^{A}. In some embodiments, each R^{Z1}, R^{Z2}, R^{Z3} and R^{Z4} is hydrogen.

In some embodiments, R^{Z1} is hydrogen or R^{A}. In some embodiments, R^{Z1} hydrogen. In some embodiments, R^{Z1} is R^{A}. In some embodiments, R^{Z1} is R^{A}, wherein R^{A} is selected from halogen, —OR^{a}, C₁-C₆ alkyl, C₁-C₆ haloalkyl, and cycloalkyl, wherein the alkyl or cycloalkyl is optionally substituted with one or more R^{A1}. In some embodiments, R^{Z1} is R^{A}, wherein R^{A} is selected from halogen, —OR^{a}, C₁-C₆ alkyl, C₁-C₆ haloalkyl, and cycloalkyl. In some embodiments, R^{A} is C₁-C₆ alkyl. In some embodiments, R^{A} is CH₃. In some embodiments, R^{A} is CH(CH₃)₂. In some embodiments, R^{A} is —OR^{a}. In some embodiments, R^{A} is —OCH₃. In some embodiments, R^{A} is —OCF₂H. In some embodiments, R^{A} is cycloalkyl. In some embodiments, R^{A} is

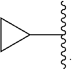 [SMILES: *C1CC1]

In some embodiments, R^{Z2} is hydrogen or R^{A}. In some embodiments, R^{Z2} is hydrogen. In some embodiments, R^{Z2} is R^{A}. In some embodiments, R^{Z2} is R^{A}, wherein R^{A} is selected from halogen, —OR^{a}, C₁-C₆ alkyl, C₁-C₆ haloalkyl, and cycloalkyl, wherein the alkyl or cycloalkyl is optionally substituted with one or more R^{A1}. In some embodiments, R^{Z2} is R^{A}, wherein R^{A} is selected from halogen, —OR^{a}, C₁-C₆ alkyl, C₁-C₆ haloalkyl, and cycloalkyl. In some embodiments, R^{A} is C₁-C₆ alkyl. In some embodiments, R^{A} is CH₃. In some embodiments, R^{A} is CH(CH₃)₂. In some embodiments, R^{A} is —OR^{a}. In some embodiments, R^{A} is —OCH₃. In some embodiments, R^{A} is —OCF₂H. In some embodiments, R^{A} is cycloalkyl. In some embodiments, R^{A} is

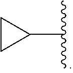 [SMILES: *C1CC1]

In some embodiments, R^{Z3} is hydrogen or R^{A}. In some embodiments, R^{Z3} is hydrogen. In some embodiments, R^{Z3} is R^{A}. In some embodiments, R^{Z3} is R^{A}, wherein R^{A} is selected from halogen, —OR^{a}, C₁-C₆ alkyl, C₁-C₆ haloalkyl, and cycloalkyl, wherein the alkyl or cycloalkyl is optionally substituted with one or more R^{A1}. In some embodiments, R^{Z3} is R^{A}, wherein R^{A} is selected from halogen, —OR^{a}, C₁-C₆ alkyl, C₁-C₆ haloalkyl, and cycloalkyl. In some embodiments, R^{A} is C₁-C₆ alkyl. In some embodiments, R^{A} is CH₃. In some embodiments, R^{A} is CH(CH₃)₂. In some embodiments, R^{A} is —OR^{a}. In some embodiments, R^{A} is —OCH₃. In some embodiments, R^{A} is —OCF₂H. In some embodiments, R^{A} is cycloalkyl. In some embodiments, R^{A} is

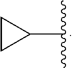 [SMILES: *C1CC1]

In some embodiments, R^{Z4} is hydrogen or R^{A}. In some embodiments, R^{Z4} is hydrogen. In some embodiments, R^{Z4} is R^{A}. In some embodiments, R^{Z4} is R^{A}, wherein R^{A} is selected from halogen, —OR^{a}, C₁-C₆ alkyl, C₁-C₆ haloalkyl, and cycloalkyl, wherein the alkyl or cycloalkyl is optionally substituted with one or more R^{A1}. In some embodiments, R^{Z4} is R^{A}, wherein R^{A} is selected from halogen, —OR^{a}, C₁-C₆ alkyl, C₁-C₆ haloalkyl, and cycloalkyl. In some embodiments, R^{A} is C₁-C₆ alkyl. In some embodiments, R^{A} is CH₃. In some embodiments, R^{A} is CH(CH₃)₂. In some embodiments, R^{A} is —OR^{a}. In some embodiments, R^{A} is —OCH₃. In some embodiments, R^{A} is —OCF₂H. In some embodiments, R^{A} is cycloalkyl. In some embodiments, R^{A} is

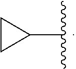 [SMILES: *C1CC1]

In some embodiments, R^{Z5} is hydrogen or R^{A}. In some embodiments, R^{Z5} is hydrogen. In some embodiments, R^{Z5} is R^{A}. In some embodiments, R^{Z5} is R^{A}, wherein R^{A} is selected from halogen, —OR^{a}, C₁-C₆ alkyl, C₁-C₆ haloalkyl, and cycloalkyl, wherein the alkyl or cycloalkyl is optionally substituted with one or more R^{A1}. In some embodiments, R^{Z5} is R^{A}, wherein R^{A} is selected from halogen, —OR^{a}, C₁-C₆ alkyl, C₁-C₆ haloalkyl, and cycloalkyl. In some embodiments, R^{A} is C₁-C₆ alkyl. In some embodiments, R^{A} is CH₃. In some embodiments, R^{A} is CH(CH₃)₂. In some embodiments, R^{A} is —OR^{a}. In some embodiments, R^{A} is —OCH₃. In some embodiments, R^{A} is —OCF₂H. In some embodiments, R^{A} is cycloalkyl. In some embodiments, R^{A} is

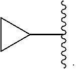 [SMILES: *C1CC1]

In some embodiments, Y¹ is N or CR^{Y1}. In some embodiments, Y¹ is N. In some embodiments, Y¹ is CR^{Y1}. In some embodiments, Y² is N or CR^{Y2}. In some embodiments, Y² is N. In some embodiments, Y² is CR^{Y2}. In some embodiments, Y³ is N or CR^{Y3}. In some embodiments, Y³ is N. In some embodiments, Y³ is CR^{Y3}. In some embodiments, Y⁴ is N or CR^{Y4}. In some embodiments, Y⁴ is N. In some embodiments, Z⁴ is CR^{Y4}.

In some embodiments, each of R^{Y1}, R^{Y2}, R^{Y3}, and R^{Z4} is independently hydrogen or R^{B}. In some embodiments, each R^{Y1}, R^{Y2}, R^{Y3} and R^{Y4} is hydrogen.

In some embodiments, R^{Y1} is hydrogen or R^{B}. In some embodiments, R^{Y1} hydrogen. In some embodiments, R^{Y1} is R^{B}. In some embodiments, R^{Y1} is R^{B}, wherein R^{B} is selected from halogen, C₁-C₆ alkyl, and C₁-C₆ haloalkyl, wherein the alkyl is optionally substituted with one or more R^{B1}. In some embodiments, R^{B} is F.

In some embodiments, R^{Y2} is hydrogen or R^{B}. In some embodiments, R^{Y2} is hydrogen. In some embodiments, R^{Y2} is R^{B}. In some embodiments, R^{ZY} is R^{B}, wherein R^{B} is selected from halogen, C₁-C₆ alkyl, and C₁-C₆ haloalkyl, wherein the alkyl is optionally substituted with one or more R^{B1}. In some embodiments, R^{B} is F.

In some embodiments, R^{Y3} is hydrogen or R^{B}. In some embodiments, R^{Y3} is hydrogen. In some embodiments, R^{Y3} is R^{B}. In some embodiments, R^{Y3} is R^{B}, wherein R^{B} is selected from halogen, C₁-C₆ alkyl, and C₁-C₆ haloalkyl, wherein the alkyl is optionally substituted with one or more R^{B1}. In some embodiments, R^{B} is F.

In some embodiments, R^{Y4} is hydrogen or R^{B}. In some embodiments, R^{Y4} is hydrogen. In some embodiments, R^{Y4} is R^{B}. In some embodiments, R^{Y4} is R^{B}, wherein R^{B} is selected from halogen, C₁-C₆ alkyl, and C₁-C₆ haloalkyl, wherein the alkyl is optionally substituted with one or more R^{B1}. In some embodiments, R^{B} is F.

In some embodiments, R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl. In some embodiments, R⁴ and R^{4′} are taken together to form an oxo. In some embodiments, R⁴ and R^{4′} are taken together with the carbon to which they are attached to form a 3-6 membered cycloalkyl or 3-6 membered heterocycloalkyl, each of which is optionally substituted with one or more R. In some embodiments, R⁴ and R^{4′} are taken together with the carbon to which they are attached to form a 3-6 membered cycloalkyl or 3-6 membered heterocycloalkyl.

In some embodiments, R⁴ is selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R⁴ is selected from hydrogen, halogen, C₁₋₆ alkyl, and C₁-C₆ haloalkyl, wherein the alkyl is optionally substituted with one or more R. In some embodiments, R⁴ is hydrogen. In some embodiments, R⁴ is halogen. In some embodiments, R⁴ is C₁₋₆ alkyl. In some embodiments, R⁴ is C₁₋₆ alkyl is optionally substituted with one or more R. In some embodiments, R⁴ is C₁-C₆ haloalkyl.

In some embodiments, R^{4′} is selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R^{4′} is selected from hydrogen, halogen, C₁₋₆ alkyl, and C₁-C₆ haloalkyl, wherein the alkyl is optionally substituted with one or more R. In some embodiments, R^{4′} is hydrogen. In some embodiments, R^{4′} is halogen. In some embodiments, R^{4′} is C₁₋₆ alkyl. In some embodiments, R^{4′} is C₁₋₆ alkyl is optionally substituted with one or more R. In some embodiments, R^{4′} is C₁-C₆ haloalkyl.

In some embodiments, R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl. In some embodiments, R⁵ and R^{5′} are taken together to form an oxo. In some embodiments, R⁵ and R^{5′} are taken together with the carbon to which they are attached to form a 3-6 membered cycloalkyl or 3-6 membered heterocycloalkyl, each of which is optionally substituted with one or more R. In some embodiments, R⁵ and R^{5′} are taken together with the carbon to which they are attached to form a 3-6 membered cycloalkyl or 3-6 membered heterocycloalkyl.

In some embodiments, R⁵ is selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R⁵ is selected from hydrogen, halogen, C₁₋₆ alkyl, and C₁-C₆ haloalkyl, wherein the alkyl is optionally substituted with one or more R. In some embodiments, R⁵ is hydrogen. In some embodiments, R⁵ is halogen. In some embodiments, R⁵ is C₁₋₆ alkyl. In some embodiments, R⁵ is C₁₋₆ alkyl is optionally substituted with one or more R. In some embodiments, R⁵ is C₁-C₆ haloalkyl.

In some embodiments, R^{5′} is selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R^{5′} is selected from hydrogen, halogen, C₁₋₆ alkyl, and C₁-C₆ haloalkyl, wherein the alkyl is optionally substituted with one or more R. In some embodiments, R^{5′} is hydrogen. In some embodiments, R^{5′} is halogen. In some embodiments, R^{5′} is C₁₋₆ alkyl. In some embodiments, R^{5′} is C₁₋₆ alkyl is optionally substituted with one or more R. In some embodiments, R^{5′} is C₁-C₆ haloalkyl.

In some embodiments, R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl. In some embodiments, R⁶ and R^{6′} are taken together to form an oxo. In some embodiments, R⁶ and R^{6′} are taken together with the carbon to which they are attached to form a 3-6 membered cycloalkyl or 3-6 membered heterocycloalkyl, each of which is optionally substituted with one or more R. In some embodiments, R⁶ and R^{6′} are taken together with the carbon to which they are attached to form a 3-6 membered cycloalkyl or 3-6 membered heterocycloalkyl.

In some embodiments, R⁶ is selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R⁶ is selected from hydrogen, halogen, C₁₋₆ alkyl, and C₁-C₆ haloalkyl, wherein the alkyl is optionally substituted with one or more R. In some embodiments, R⁶ is hydrogen. In some embodiments, R⁶ is halogen. In some embodiments, R⁶ is C₁₋₆ alkyl. In some embodiments, R⁶ is C₁₋₆ alkyl is optionally substituted with one or more R. In some embodiments, R⁶ is C₁-C₆ haloalkyl.

In some embodiments, R^{6′} is selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R. In some embodiments, R^{6′} is selected from hydrogen, halogen, C₁₋₆ alkyl, and C₁-C₆ haloalkyl, wherein the alkyl is optionally substituted with one or more R. In some embodiments, R^{6′} is hydrogen. In some embodiments, R^{6′} is halogen. In some embodiments, R^{6′} is C₁₋₆ alkyl. In some embodiments, R^{6′} is C₁₋₆ alkyl is optionally substituted with one or more R. In some embodiments, R^{6′} is C₁-C₆ haloalkyl.

In some embodiments, R^{A} is selected from halogen, —CN, —NO₂, —OH, —OR^{a}, —OC(═O)R^{a}, —OC(═O)OR, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{A1}. In some embodiments, R^{A} is selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl. In some embodiments, two R^{A} are taken together with the intervening atoms to which they are attached to form a cycloalkyl, heterocycloalkyl, aryl, or heteroaryl, wherein each of which independently optionally substituted with one or more R^{A1}. In some embodiments, R^{A} is selected from halogen, —OR^{a}, C₁-C₆ alkyl, C₁-C₆ haloalkyl, and cycloalkyl. In some embodiments, R^{A} is C₁-C₆ alkyl. In some embodiments, R^{A} is CH₃. In some embodiments, R^{A} is CH(CH₃)₂. In some embodiments, R^{A} is —OR^{a}. In some embodiments, R^{A} is —OCH₃. In some embodiments, R^{A} is —OCF₂H. In some embodiments, R^{A} is cycloalkyl. In some embodiments, R^{A} is

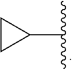 [SMILES: *C1CC1]

In some embodiments, R^{A} is not C₁₋₆ alkoxyl. In some embodiments, R^{A} is not C₁. In some embodiments, R^{A} is not halogen.

In some embodiments, each R^{A} is independently selected from halogen, OH, —NO₂, oxo, —CN, C₁₋₆ alkyl, C₁₋₆ alkoxyl, C₁₋₆ haloalkyl, C₁₋₆ heteroalkyl, and C₃₋₆ cycloalkyl. In some embodiments, each R^{A} is independently selected from halogen, C₁₋₆ alkyl, C₁₋₆ alkoxyl, C₁₋₆ haloalkyl, and C₃₋₆ cycloalkyl. In some embodiments, each R^{A} is independently selected from C₁₋₆ alkyl, C₁₋₆ alkoxyl, C₁₋₆ haloalkoxyl, C₁₋₆ haloalkyl, and C₃₋₆ cycloalkyl. In some embodiments, each R^{A} is independently selected from halogen, C₁₋₆ alkyl, C₁₋₆ alkoxyl, and C₃₋₆ cycloalkyl, wherein the alkyl, alkoxyl and cycloalkyl is optionally substituted with one or more halogen (e.g., 1-3 fluorine). In some embodiments, each R^{A} is independently selected from methyl, ethyl, propyl, butyl, —O-methyl, —O-ethyl, —O-propyl, —O-butyl, cyclopropyl, CN, OH, —O—CHF₂, —O—CH₂F, CHF₂, CH₂F, and CF₃. In some embodiments, R^{A} is halogen. In some embodiments, R^{A} is —NO₂. In some embodiments, R^{A} is oxo. In some embodiments, R^{A} is —CN. In some embodiments, R^{A} is optionally substituted C₁₋₆ alkyl. In some embodiments, R^{A} is C₁₋₆ alkyl. In some embodiments, R^{A} is C₂ alkyl. In some embodiments, R^{A} is C₃ alkyl. In some embodiments, R^{A} is optionally substituted C₁₋₆ heteroalkyl. In some embodiments, R^{A} is C₃ heteroalkyl. In some embodiments, R^{A} is optionally substituted C₃₋₈ cycloalkyl. In some embodiments, R^{A} is C₃ cycloalkyl. In some embodiments, R^{A} is optionally substituted C₂₋₇ heterocycloalkyl. In some embodiments, R^{A} is C₂ heterocycloalkyl.

In some embodiments, each R^{A} is independently substituted with one or more substituents independently selected from: halogen, —OH, —NO₂, amino, —CN, C₁₋₆ alkoxyl, C₁₋₆ alkyl, C₁₋₆ haloalkyl, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, amino, —NO₂, oxo, —CN, C₁₋₆ alkyl, C₁₋₆ alkoxy, and C₁₋₆ haloalkyl.

In some embodiments, R^{A} is independently substituted with one or more substituents independently selected from: halogen, —OH, —NO₂, amino, —CN, C₁₋₆ alkoxyl, C₁₋₆ alkyl, C₁₋₆ haloalkyl, C₃₋₆ carbocycle, and 3- to 6-membered heterocycle, wherein the C₃₋₆ carbocycle and 3- to 6-membered heterocycle is optionally substituted with one or more substituents independently selected from halogen, —OH, amino, —NO₂, oxo, —CN, C₁₋₆ alkyl, C₁₋₆ alkoxy, and C₁₋₆ haloalkyl.

In some embodiments, each of R^{A} is independently selected from halogen, —NO₂, oxo, CN, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₈ cycloalkyl, or optionally substituted C₂₋₇ heterocycloalkyl. In some embodiments, R^{A} is halogen. In some embodiments, R^{A} is —NO₂. In some embodiments, R^{A} is oxo. In some embodiments, R^{A} is CN. In some embodiments, R^{A} is optionally substituted C₁₋₆ alkyl. In some embodiments, R^{A} is optionally substituted C₁₋₃ alkyl. In some embodiments, R^{A} is methyl, ethyl, propyl, iso-propyl, n-butyl, iso-butyl, sec-butyl, t-butyl, —CF₃, —CH₂CF₃, or —CH₂CH₂F. In some embodiments, R^{A} is optionally substituted C₂₋₆ alkenyl. In some embodiments, R^{A} is optionally substituted C₂₋₆ alkynyl. In some embodiments, R^{A} is optionally substituted C₁₋₆ heteroalkyl. In some embodiments, R^{A} is optionally substituted C₃₋₈ cycloalkyl. In some embodiments, R^{A} is optionally substituted C₃₋₆ cycloalkyl, e.g., cyclopropyl. In some embodiments, R^{A} is

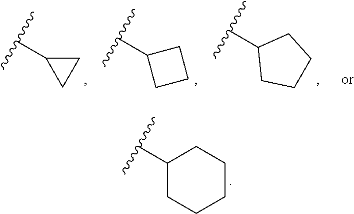

In some embodiments, R^{A} is optionally substituted C₂₋₇ heterocycloalkyl. In some embodiments, R^{A} is optionally substituted C₂₋₅ heterocycloalkyl. In some embodiments, R^{A} is —O—C₁₋₃ alkyl. In some embodiments, R^{A} is —OCH₃, —OCH₂CH₃, —OCH₂OMe, —OCH₂CH₂OH, —OC(CH₃)₃, or —OCH₂CH₂OCH₃. In some embodiments, R^{A} is —OCH₃. In some embodiments, R^{A} is

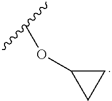 [SMILES: *OC1CC1]

In some embodiments, —OCH₃ is —OCD₃.

In some embodiments, each R^{A} is independently OH, C₁₋₆ alkoxyl (e.g., —OCH₃), C₁₋₆ alkyl, C₁₋₆ haloalkyl, or C₃-C₆ cycloalkyl (e.g., cyclopropyl).

In some embodiments, each R^{A} is independently OH, C₁₋₃ alkyl, C₁₋₃ alkoxyl (e.g., —OCH₃), C₁₋₃ haloalkyl, or C₃-C₆ cycloalkyl (e.g., cyclopropyl). In some embodiments, each R^{A} is independently C₁₋₃ alkoxyl (e.g., —OCH₃) or C₃-C₆ cycloalkyl (e.g., cyclopropyl). In some embodiments, each R^{A} is independently —OCH₃, C₁₋₃ alkyl, C₁₋₃ haloalkyl, or cyclopropyl. In some embodiments, each R^{A} is independently C₁₋₃ alkoxyl, C₁₋₃ alkyl, C₁₋₃ haloalkyl, or cyclopropyl. In some embodiments, —OCH₃ is —OCD₃.

In some embodiments, R^{A1} is selected from halogen, —CN, —NO₂, —OH, —OR^{a}, —OC(═O)R^{a}, —OC(═O)OR, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl; wherein each of the alkyl, heteroalkyl, alkenyl, alkynyl, cycloalkyl, heterocycloalkyl, aryl, and heteroaryl is independently optionally substituted with one or more R. In some embodiments, R^{A1} is selected from halogen, —CN, —NO₂, —OH, —OR^{a}, —OC(═O)R^{a}, —OC(═O)OR, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl. In some embodiments, R^{A1} is halogen, C₁-C₆ alkyl, cycloalkyl, wherein the alkyl or cycloalkyl is optionally substituted with one or more R.

In some embodiments of a compound of Formula (I), ring A aryl or heteroaryl. In some embodiments, ring A is phenyl or 5-6 membered monocyclic heteroaryl. In some embodiments, ring A is phenyl or 5-6 membered monocyclic heteroaryl. In some embodiments, Ring A is pyridine, pyrimidine, pyrazine, pyridazine, triazine, imidazole, pyrazole, triazole, oxazole, isoxazole, thiophene, or thiazole.

In some embodiments,

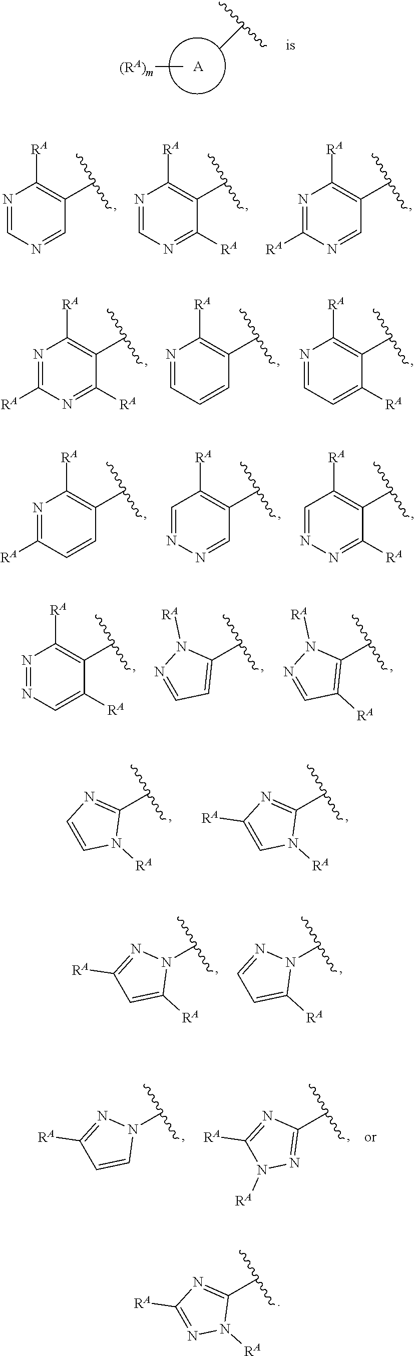

In some embodiments,

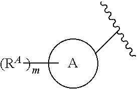

is selected from:

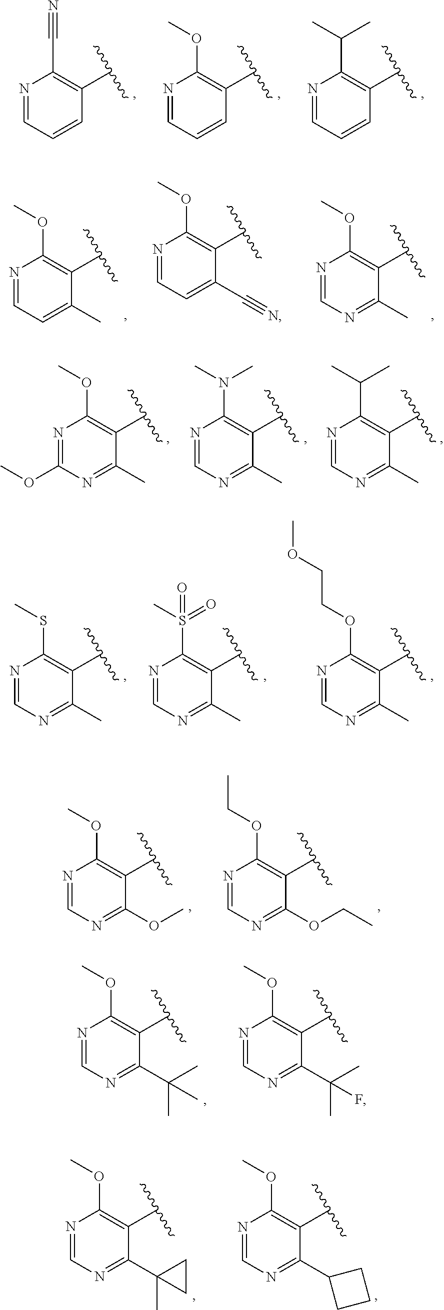

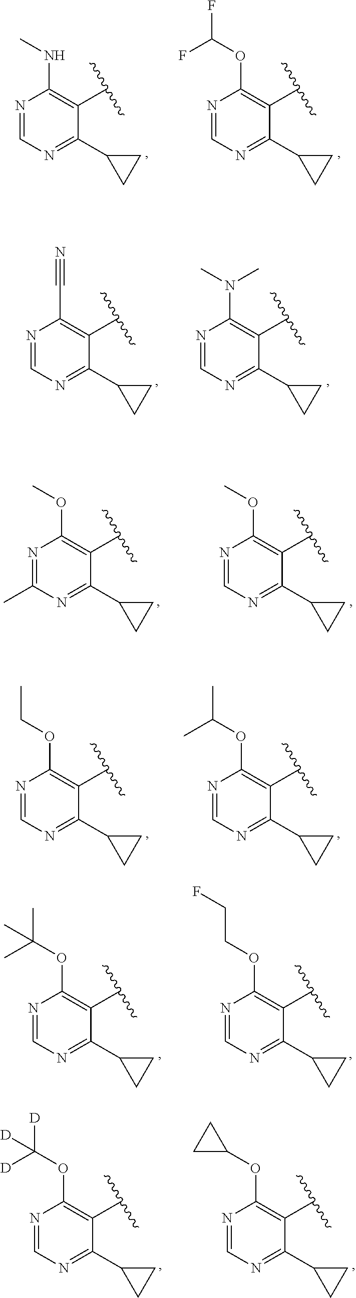

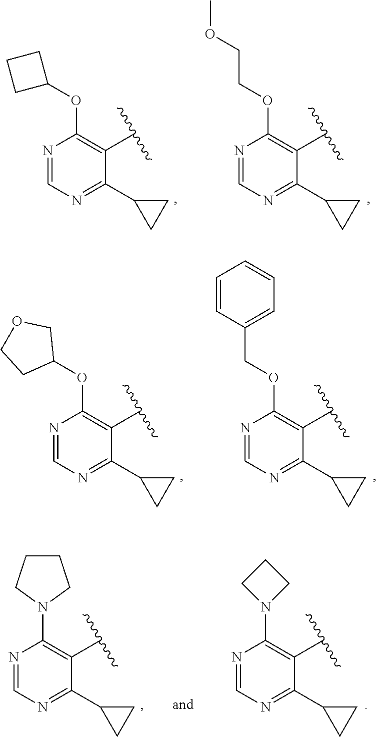 [SMILES: *c1c(OC2CCC2)ncnc1C1CC1 ; *c1c(OCCOC)ncnc1C1CC1 ; *c1c(OC2CCOC2)ncnc1C1CC1 ; *c1c(OCc2ccccc2)ncnc1C1CC1 ; *c1c(C2CC2)ncnc1N1CCCC1 ; *c1c(C2CC2)ncnc1N1CCC1]

In some embodiments,

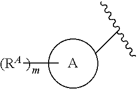

is selected from

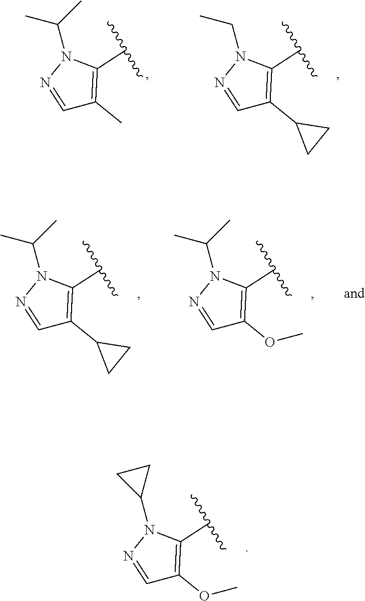

In some embodiments,

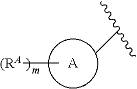

is selected from:

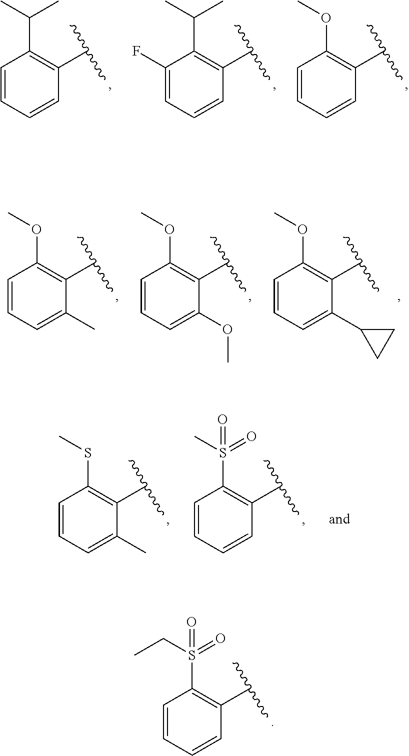

In some embodiments,

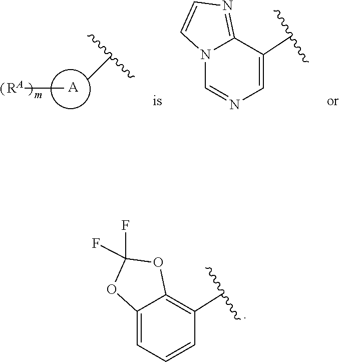

In some embodiments,

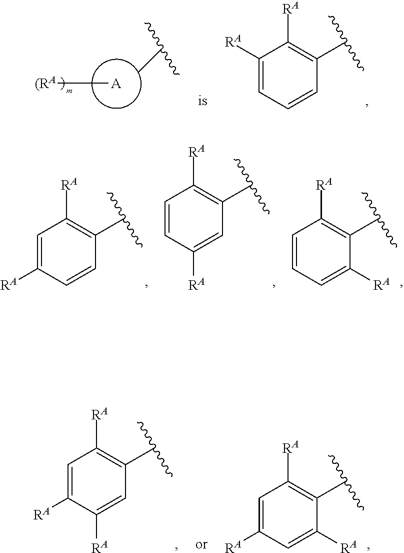

In some embodiments,

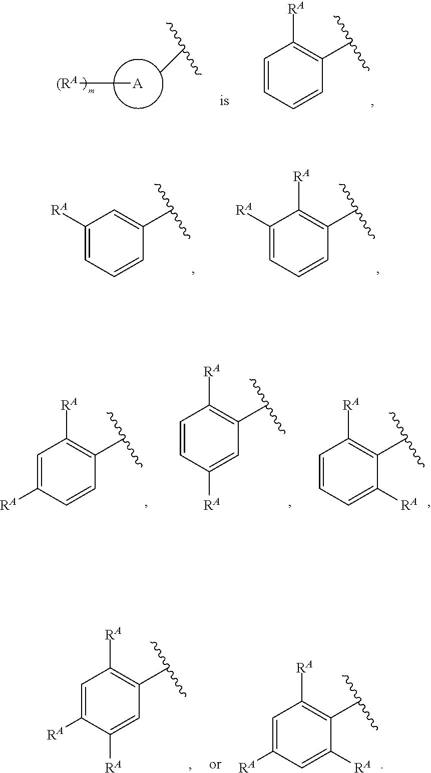

In some embodiments, ring A is unsubstituted aryl (e.g., phenyl). In some embodiments, ring A is aryl (e.g., phenyl) that is optionally substituted with 1 to 5 R^{A}. In some embodiments, ring A is aryl (e.g., phenyl) that is substituted with 1 R^{A}. In some embodiments, ring A is aryl (e.g., phenyl) that is substituted with 2 R^{A}. In some embodiments, and (IVa-2), ring A is aryl (e.g., phenyl) that is substituted with 3 R^{A}. In some embodiments, ring A is aryl (e.g., phenyl) that is substituted with 4 R^{A}. In some embodiments, ring A is aryl (e.g., phenyl) that is substituted with 5 R^{A}.

In some embodiments, ring A is monocyclic heteroaryl or bicyclic heteroaryl. In some embodiments, ring A is monocyclic heteroaryl. In some embodiments, ring A is bicyclic heteroaryl.

In some embodiments, ring A is phenyl, naphthyl, monocyclic heteroaryl, or bicyclic heteroaryl. In some embodiments, ring A is phenyl. In some embodiments, ring A is naphthyl. In some embodiments, ring A is monocyclic heteroaryl. In some embodiments, ring A is or bicyclic heteroaryl.

In some embodiments, ring A is naphthyl. In some embodiments, ring A is 5 or 6 membered monocyclic heteroaryl. In some embodiments, ring A is a 6 membered monocyclic heteroaryl containing 1-3 heteroatoms.

In some embodiments, ring A is a 5 membered monocyclic heteroaryl. In some embodiments, ring A is a 6 membered monocyclic heteroaryl. In some embodiments, ring A is a 6 membered monocyclic heteroaryl containing 1-3 heteroatoms. In some embodiments, ring A is pyridine, pyrimidine, pyrazine, pyridazine, triazine, imidazole, pyrazole, triazole, oxazole, isoxazole, or thiophene. In some embodiments,

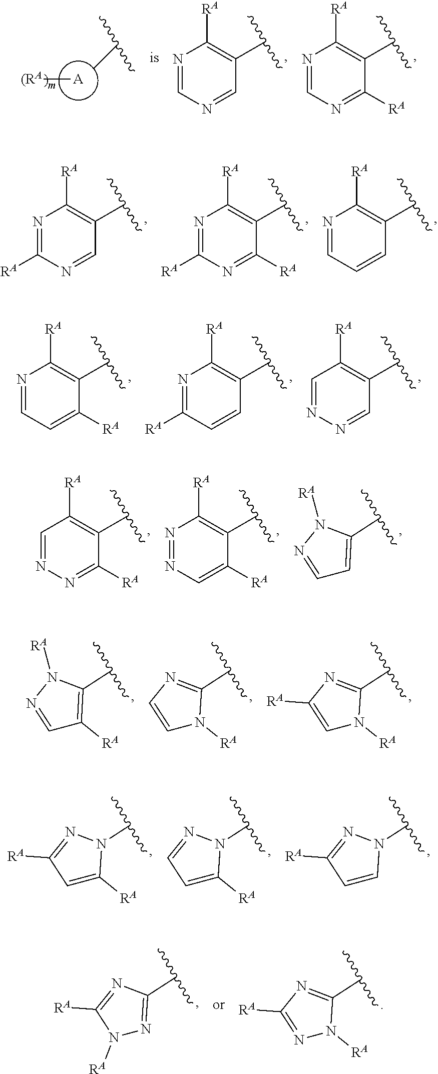

In some embodiments,

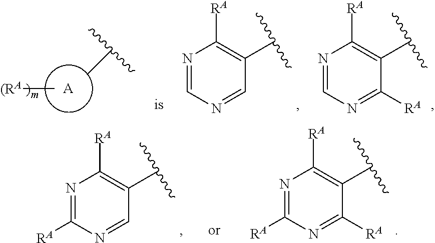

In some embodiments,

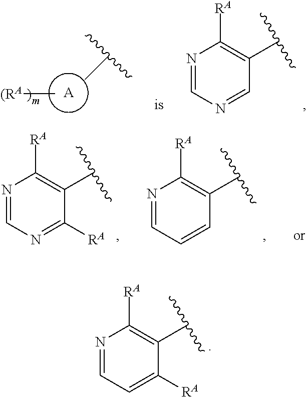

In some embodiments,

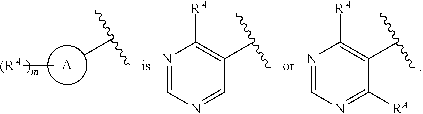

In some embodiments,

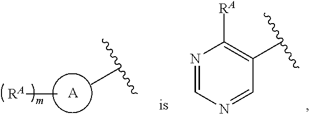

In some embodiments,

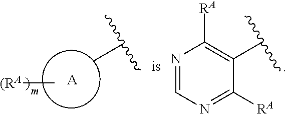

In some embodiments,

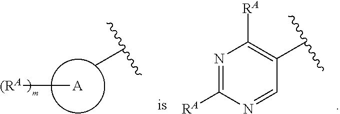

In some embodiments,

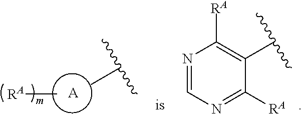

In some embodiments,

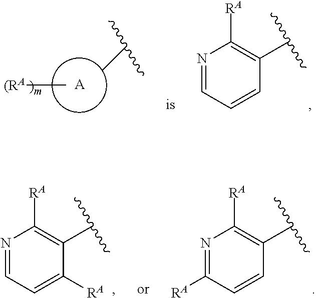

In some embodiments,

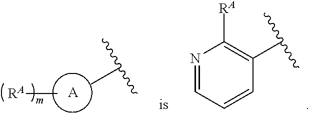

In some embodiments,

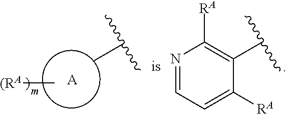

In some embodiments,

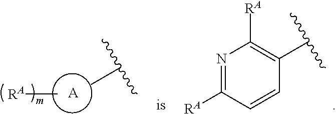

In some embodiments,

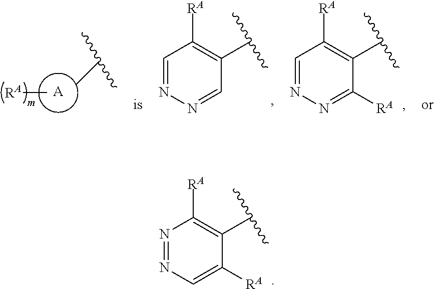

In some embodiments,

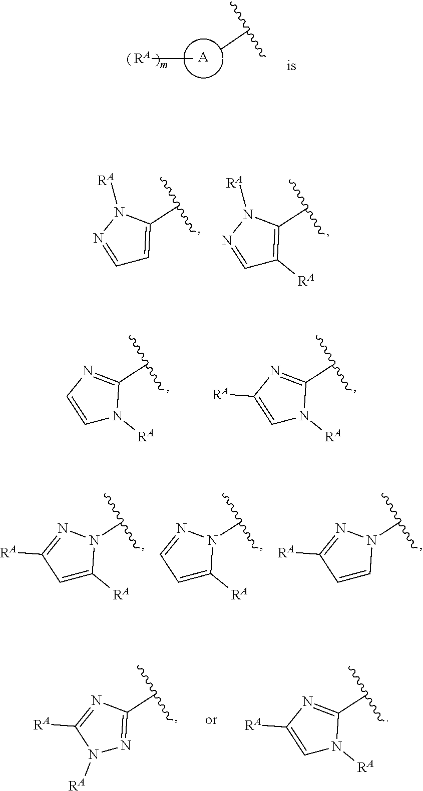

In some embodiments, ring A is bicyclic heteroaryl. In some embodiments, ring A is fused 5-6, 6-6, or 6-5 bicyclic heteroaryl.

In some embodiments,

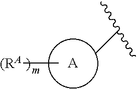

is selected from

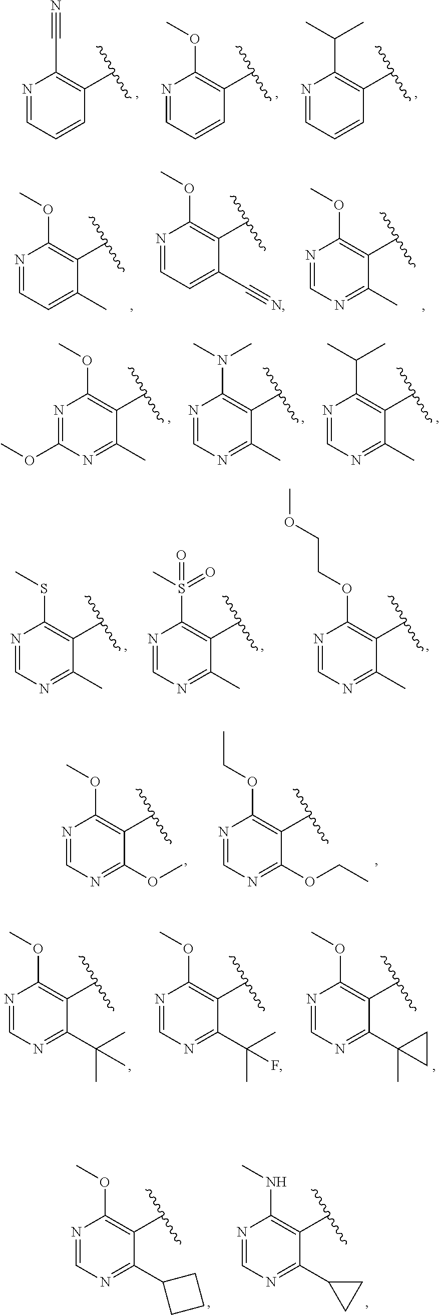

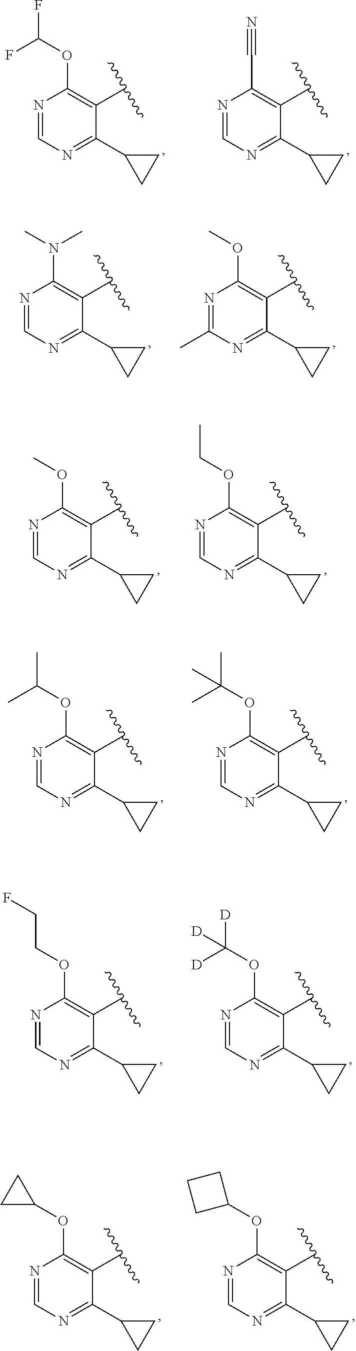

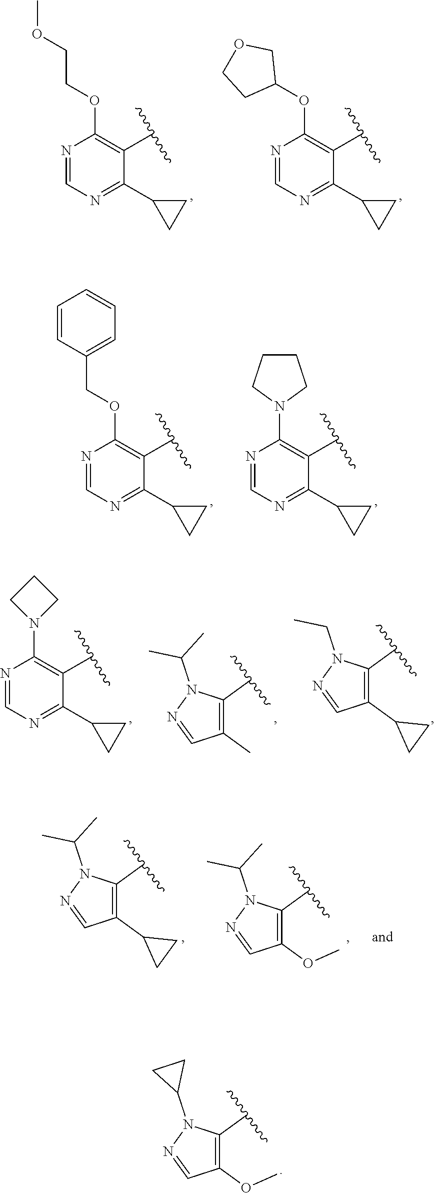

In some embodiments,

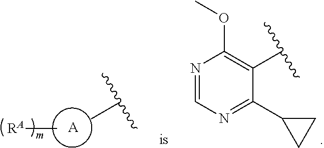

In some embodiments,

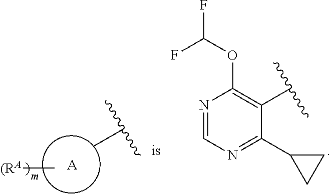

In some embodiments,

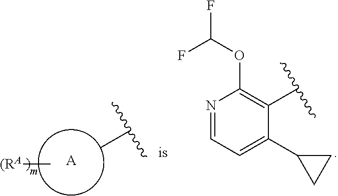

In some embodiments,

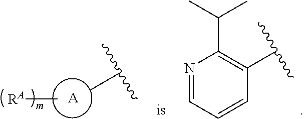

In some embodiments,

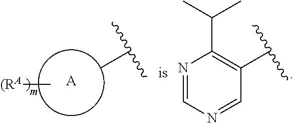

In some embodiments,

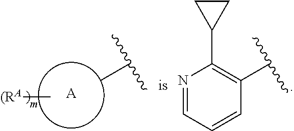

In some embodiments,

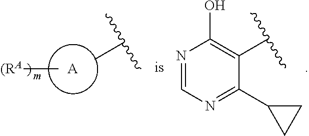

In some embodiments,

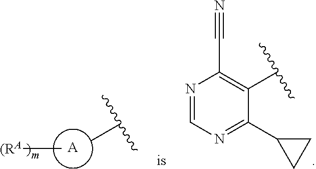

In some embodiments,

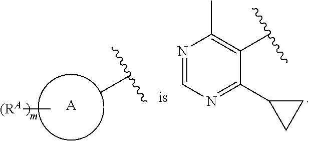

In some embodiments,

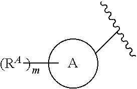

is selected from:

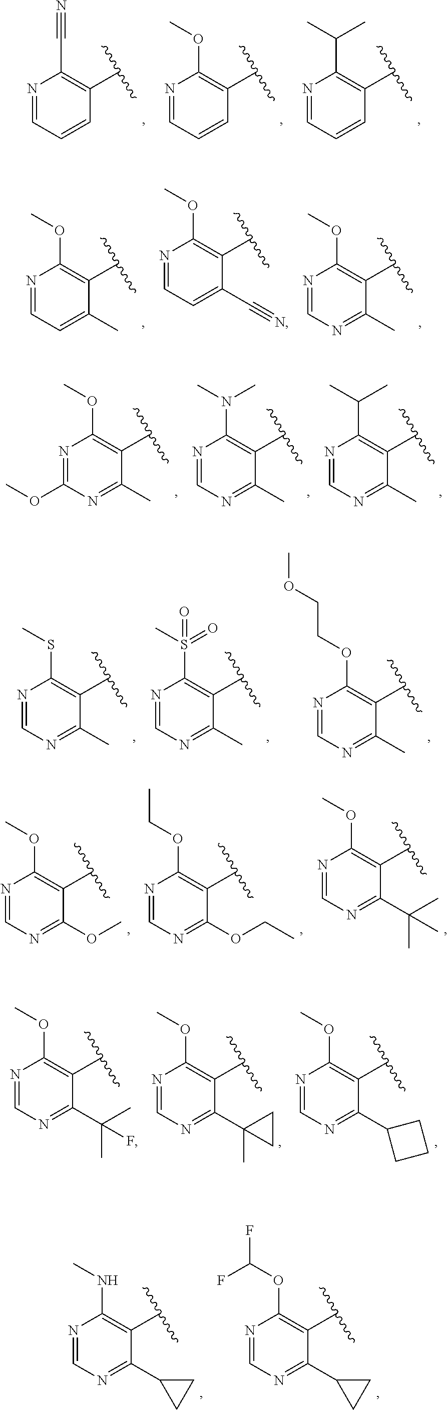

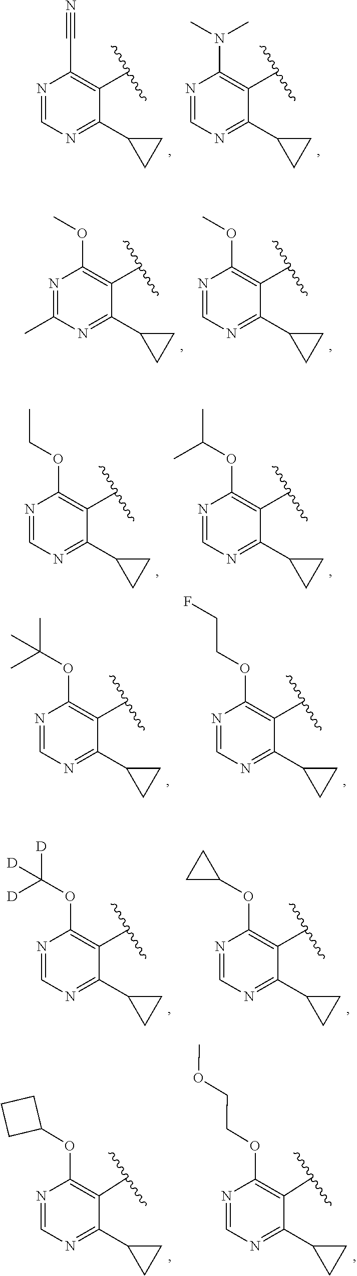

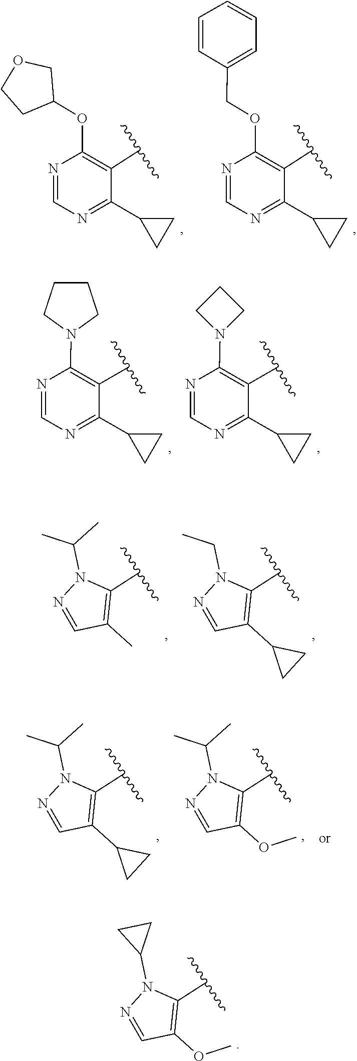

In some embodiments,

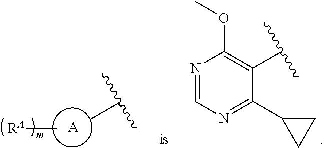

In some embodiments,

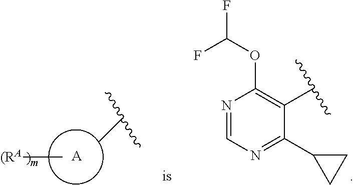

In some embodiments,

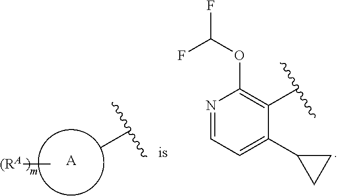

In some embodiments,

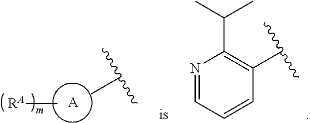

In some embodiments,

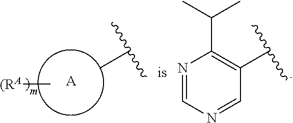

In some embodiments,

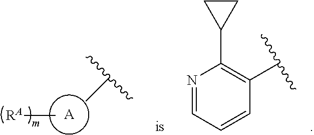

In some embodiments,

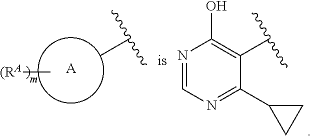

In some embodiments,

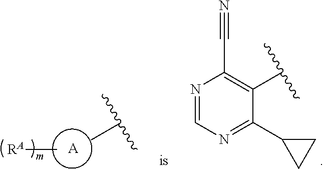

In some embodiments,

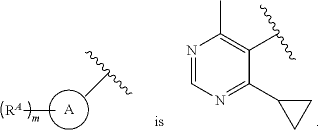

In some embodiments,

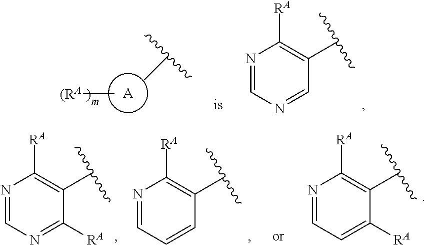

In some embodiments, ring B phenyl, phenyl isostere, or heteroaryl. In some embodiments, ring B is phenyl. In some embodiments, ring B is phenyl isostere. In some embodiments, the phenyl isostere is cubane. In some embodiments, ring B is heteroaryl. In some embodiments, ring B is 5-6 membered monocyclic heteroaryl. In some embodiments, ring B is pyridine, pyrimidine, pyrazine, pyridazine, triazine, imidazole, pyrazole, triazole, oxazole, isoxazole, thiophene, or thiazole. In some embodiments, ring B is 5-6 membered monocyclic heteroaryl. In some embodiments, ring B is pyridine. In some embodiments, ring B is pyrimidine. In some embodiments, ring B is pyrazine. In some embodiments, ring B is pyridazine. In some embodiments, ring B is triazine. In some embodiments, ring B is imidazole. In some embodiments, ring B is pyrazole. In some embodiments, ring B is triazole. In some embodiments, ring B is oxazole. In some embodiments, ring B is isoxazole. In some embodiments, ring B is thiophene. In some embodiments, ring B is thiazole.

In some embodiments,

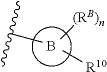

is selected from:

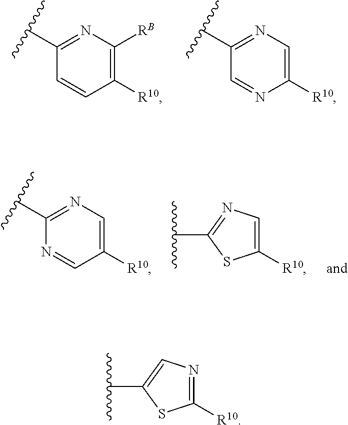

In some embodiments, R¹⁰ is cycloalkyl, heterocycloalkyl, aryl, or heteroaryl, each of which is independently optionally substituted with one or more R^{B1}. In some embodiments, R¹⁰ is heteroaryl, wherein the heteroaryl is optionally substituted with one or more R^{B1}. In some embodiments, R¹⁰ is an optionally substituted 5 membered monocyclic heteroaryl with 1 to 4 heteroatoms selected from N, O, S, and P. In some embodiments, R¹⁰ is imidazole, pyrazole, triazole, or tetrazole, each of which optionally substituted. In some embodiments, R¹⁰ is optionally substituted fused 5-6, 6-6 or 6-5 heteroaryl. In some embodiments, R¹⁰ is imidazole, pyrazole, triazole, or tetrazole, each of which optionally substituted. In some embodiments, R¹⁰ is optionally substituted fused 5-6, 6-6 or 6-5 heteroaryl.

In some embodiments, R¹⁰ is selected from:

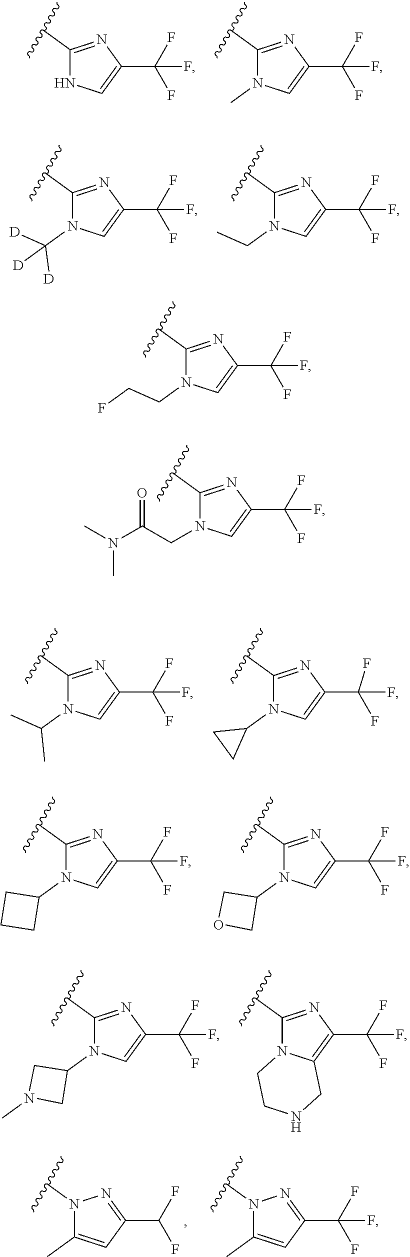

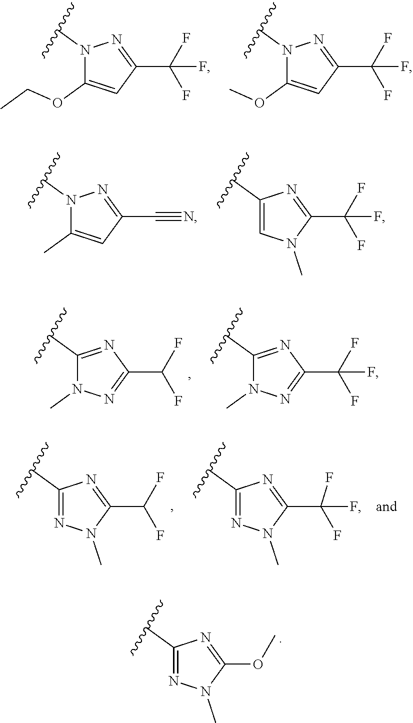

In some embodiments, R¹⁰ is selected from:

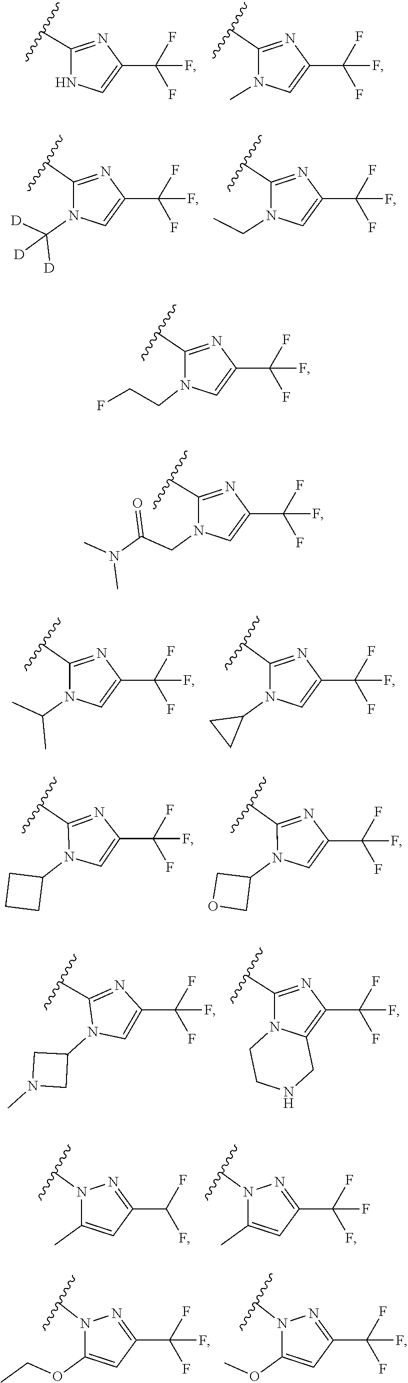

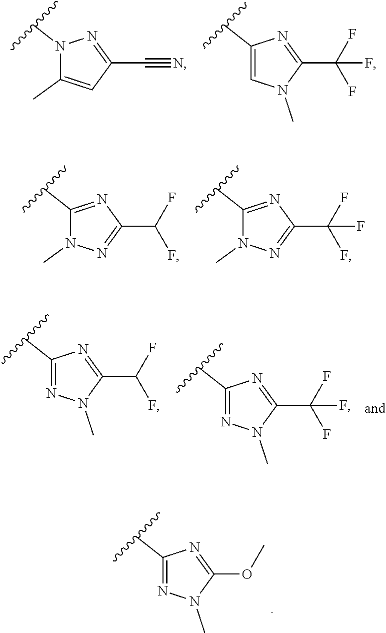

In some embodiments, R¹⁰ is

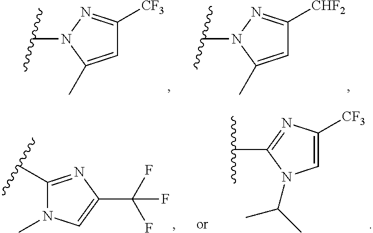

In some embodiments, R¹⁰ is

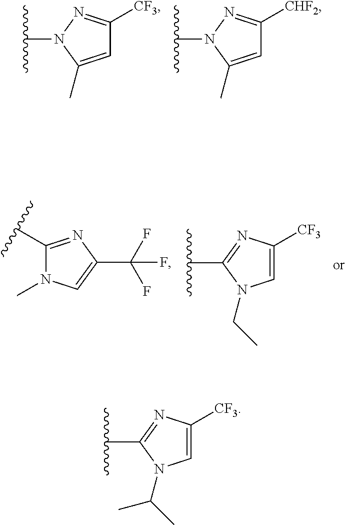

In some embodiments, R¹⁰ is

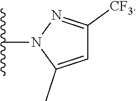 [SMILES: *n1nc(C(F)(F)F)cc1C]

In some embodiments, R¹⁰ is

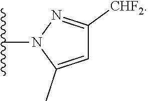 [SMILES: *n1nc(C(F)F)cc1C]

In some embodiments, R¹⁰ is

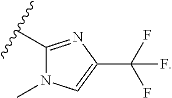 [SMILES: *c1nc(C(F)(F)F)cn1C]

In some embodiments, R¹⁰ is

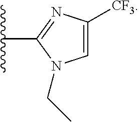 [SMILES: *c1nc(C(F)(F)F)cn1CC]

In some embodiments, R¹⁰ is

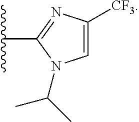 [SMILES: *c1nc(C(F)(F)F)cn1C(C)C]

In some embodiments, R¹⁰ is optionally substituted C₃₋₈ cycloalkyl or optionally substituted phenyl. In some embodiments, R¹⁰ is optionally substituted C₂₋₉ heterocycloalkyl, optionally substituted monocyclic heteroaryl, or optionally substituted bicyclic heteroaryl, each of which containing 1-4 heteroatoms selected from O, S, N, P, and Si. In some embodiments, R¹⁰ is optionally substituted C₃₋₈ cycloalkyl. In some embodiments, R¹⁰ is C₃ cycloalkyl. In some embodiments, R¹⁰ is C₅ cycloalkyl. In some embodiments, R¹⁰ is C₆ cycloalkyl. In some embodiments, R¹⁰ is optionally substituted phenyl. In some embodiments, R¹⁰ is optionally substituted C₂-9 heterocycloalkyl. In some embodiments, R¹⁰ is C₃ heterocycloalkyl. In some embodiments, R¹⁰ is C₅ heterocycloalkyl. In some embodiments, R¹⁰ is C₆ heterocycloalkyl. In some embodiments, R¹⁰ is optionally substituted monocyclic heteroaryl. In some embodiments, R¹⁰ is optionally substituted bicyclic heteroaryl. In some embodiments, R¹⁰ is imidazole, pyrazole, triazole, or tetrazole, each of which optionally substituted. In some embodiments, R¹⁰ is imidazole. In some embodiments, R¹⁰ is pyrazole. In some embodiments, R¹⁰ is triazole. In some embodiments, R¹⁰ is tetrazole. In some embodiments, R¹⁰ is optionally substituted fused 5-6, 6-6 or 6-5 heteroaryl. In some embodiments, R¹⁰ is substituted with halogen. In some embodiments, R¹⁰ is substituted with —OR¹¹. In some embodiments, R¹⁰ is substituted with —NO₂. In some embodiments, R¹⁰ is substituted with oxo. In some embodiments, R¹⁰ is substituted with —CN. In some embodiments, R¹⁰ is substituted with optionally substituted C₁₋₆ haloalkyl. In some embodiments, R¹⁰ is substituted with optionally substituted C₁₋₆ alkyl. In some embodiments, R¹⁰ is substituted with optionally substituted C₁₋₆ aminoalkyl.

In some embodiments, R¹⁰ is optionally substituted monocyclic heteroaryl, or optionally substituted bicyclic heteroaryl. In some embodiments, R¹⁰ is optionally substituted monocyclic heteroaryl (e.g., 5 membered heteroaryl). In some embodiments, R¹⁰ is substituted monocyclic heteroaryl.

In some embodiments, R¹⁰ is

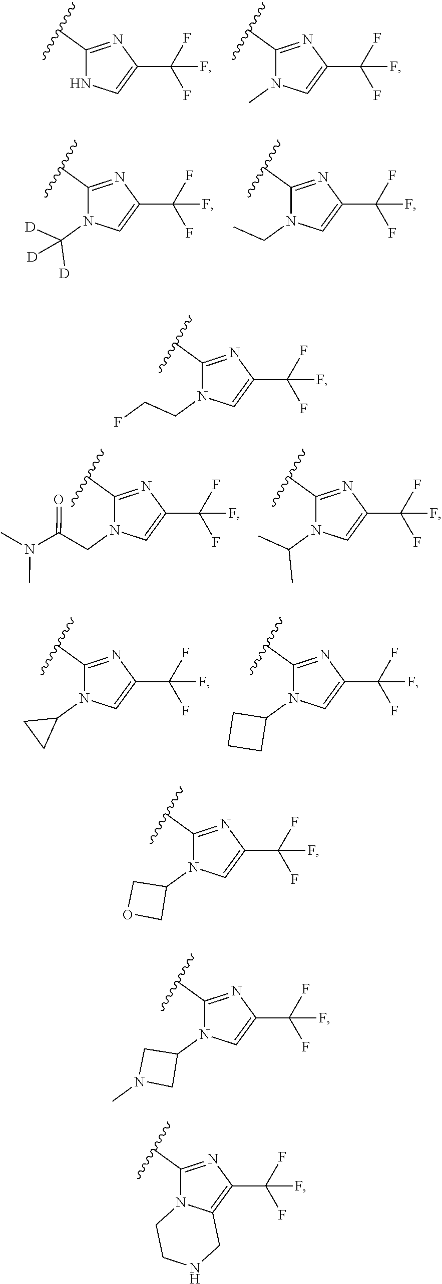

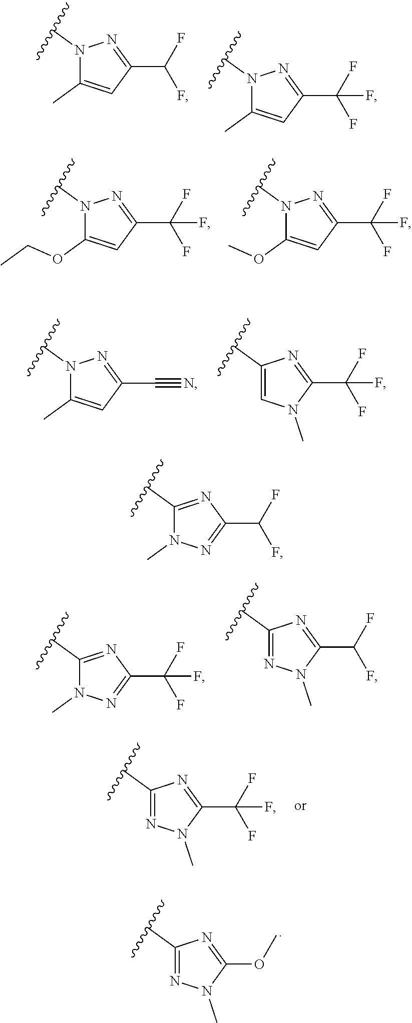

In some embodiments, R¹⁰ is

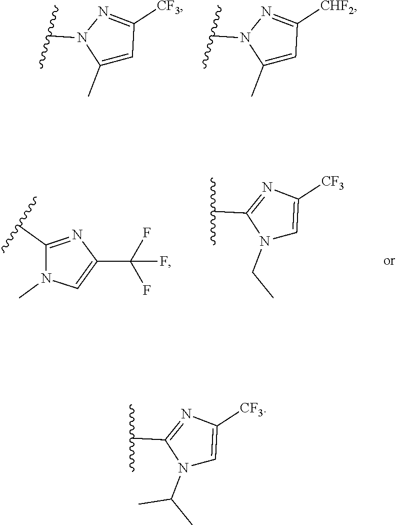

In some embodiments, R¹⁰ is

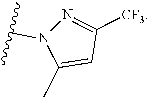 [SMILES: *n1nc(C(F)(F)F)cc1C]

In some embodiments, R¹⁰ is

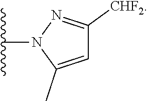 [SMILES: *n1nc(C(F)F)cc1C]

In some embodiments, R¹⁰ is

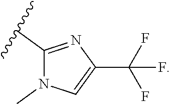 [SMILES: *c1nc(C(F)(F)F)cn1C]

In some embodiments, R¹⁰ is

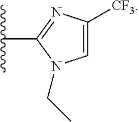 [SMILES: *c1nc(C(F)(F)F)cn1CC]

In some embodiments, R¹⁰ is

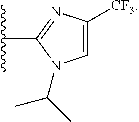 [SMILES: *c1nc(C(F)(F)F)cn1C(C)C]

In some embodiments, R¹⁰ is 5 membered heteroaryl optionally substituted with one or more substituents selected from C₁0.3 haloalkyl and C₁₋₃ alkyl

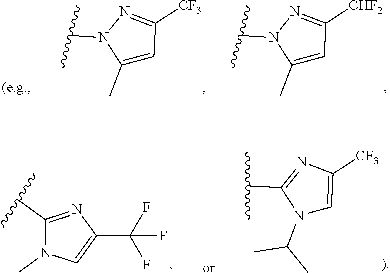

In some embodiments, R¹⁰ is substituted with 1 or 2 substituents selected from C₁₋₃ haloalkyl and C₁₋₃ alkyl.

In some embodiments, R¹⁰ is optionally substituted C₃₋₈ cycloalkyl, optionally substituted C₂₋₉ heterocycloalkyl, optionally substituted naphthyl, optionally substituted phenyl, optionally substituted monocyclic heteroaryl, or optionally substituted bicyclic heteroaryl. In some embodiments, R¹⁰ is optionally substituted C₃₋₈ cycloalkyl. In some embodiments, R¹⁰ is optionally substituted C₂₋₉ heterocycloalkyl. In some embodiments, R¹⁰ is optionally substituted 5-6 membered heterocycloalkyl. In some embodiments, R¹⁰ is optionally substituted naphthyl. In some embodiments, R¹⁰ is optionally substituted phenyl. In some embodiments, R¹⁰ is optionally substituted monocyclic heteroaryl. In some embodiments, R¹⁰ is optionally substituted bicyclic heteroaryl.

In some embodiments, R¹⁰ is optionally substituted 5 membered monocyclic heteroaryl with 1 to 4 heteroatoms selected from N, O, S and P. In some embodiments, R¹⁰ is imidazole, pyrazole, triazole, or tetrazole, each of which optionally substituted. In some embodiments, R¹⁰ is optionally substituted fused 5-6, 6-6 or 6-5 heteroaryl.

In some embodiments, R¹⁰ is optionally substituted with one or more substituents independently selected from halogen, —OR^{a}, —NO₂, oxo, —CN, optionally substituted C₁₋₆ haloalkyl, optionally substituted C₁₋₆ alkyl, optionally substituted C₁₋₆ aminoalkyl, optionally substituted C₁₋₆ hydroxyalkyl, optionally substituted C₁₋₆ heteroalkyl, optionally substituted C₃₋₈ cycloalkyl, and optionally substituted C₂₋₇ heterocycloalkyl. In some embodiments, R¹⁰ is optionally substituted with one or more substituents independently selected from halogen, —OR¹¹, —NO₂, oxo, —CN, C₁₋₃ haloalkyl, C₁₋₃ alkyl, C₁₋₃ aminoalkyl, C₁₋₃ hydroxyalkyl, optionally substituted C₁₋₄ heteroalkyl (e.g., —CH₂C(═O)N(CH₃)₂), optionally substituted C₃₋₆cycloalkyl, and optionally substituted C₂₋₅ heterocycloalkyl. In some embodiments, R¹⁰ is optionally substituted C₃₋₈ cycloalkyl. In some embodiments, R¹⁰ is C₃ cycloalkyl. In some embodiments, R¹⁰ is C₅ cycloalkyl. In some embodiments, R¹⁰ is C₆ cycloalkyl. In some embodiments, R¹⁰ is optionally substituted phenyl. In some embodiments, R¹⁰ is optionally substituted C₂₋₉ heterocycloalkyl. In some embodiments, R¹⁰ is C₃ heterocycloalkyl. In some embodiments, R¹⁰ is C₅ heterocycloalkyl. In some embodiments, R¹⁰ is C₆ heterocycloalkyl. In some embodiments, R¹⁰ is optionally substituted 5-6 membered heterocycloalkyl or heteroaryl. In some embodiments, R¹⁰ is optionally substituted monocyclic heteroaryl. In some embodiments, R¹⁰ is optionally substituted bicyclic heteroaryl. In some embodiments, R¹⁰ is imidazole, pyrazole, triazole, or tetrazole, each of which optionally substituted. In some embodiments, R¹⁰ is imidazole. In some embodiments, R¹⁰ is pyrazole. In some embodiments, R^{B} is triazole. In some embodiments, R¹⁰ is tetrazole.

In some embodiments, R^{B} is selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{B1}. In some embodiments, R^{B} is selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl. In some embodiments, R^{B} is halogen, C₁-C₆ alkyl, or C₁-C₆ haloalkyl, wherein the alkyl is optionally substituted with one or more R^{B1}. In some embodiments, R^{B} is halogen. In some embodiments, R^{B} is F. In some embodiments, R^{B} is C₁-C₆ alkyl optionally substituted with one or more R^{B1}. In some embodiments, R^{B} is C₁-C₆ alkyl. In some embodiments, R^{B} is C₁-C₆ haloalkyl.

In some embodiments, R^{B1} is selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl; wherein each of the alkyl, heteroalkyl, alkenyl, alkynyl, cycloalkyl, heterocycloalkyl, aryl, and heteroaryl is independently optionally substituted with one or more R. In some embodiments, R^{B1} is selected from halogen, —CN, —NO₂, —OH, —OR^{a}, —OC(═O)R^{a}, —OC(═O)OR, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl. In some embodiments, R^{B1} is halogen, C₁-C₆ alkyl, or C₁-C₆ haloalkyl; wherein alkyl is optionally substituted with one or more R. In some embodiments, R^{B1} is halogen. In some embodiments, R^{B1} is C₁-C₆ alkyl optionally substituted with one or more R. In some embodiments, R^{B1} is C₁-C₆ alkyl. In some embodiments, R^{B1} is CH₃. In some embodiments, R^{B1} is CH₂CH₃. In some embodiments, R^{B1} is CH(CH₃)₂. In some embodiments, R^{B1} is C₁-C₆ haloalkyl. In some embodiments, R^{B1} is CF₃.

In some embodiments, m is 0, 1, 2, 3, 4, or 5. In some embodiments, m is 1, 2, 3, 4, or 5. In some embodiments, m is 0, 1, 2, 3, or 4. In some embodiments, m is 0. In some embodiments, m is 1. In some embodiments, m is 2. In some embodiments, m is 3. In some embodiments, m is 4. In some embodiments, m is 5.

In some embodiments, n is 0, 1, 2, 3, or 4. In some embodiments, n is 1, 2, 3, or 4. In some embodiments, n is 0, 1, 2, or 3. In some embodiments, n is 0. In some embodiments, n is 1. In some embodiments, n is 2. In some embodiments, n is 3. In some embodiments, n is 4.

In some embodiments, R^{a} is C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is optionally substituted with one or more R. In some embodiments, R^{a} is hydrogen, C₁-C₆ alkyl, C₁-C₆ haloalkyl, C₁-C₆ hydroxyalkyl, C₁—C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl). In some embodiments, R^{a} is C₁-C₆ alkyl, C₁-C₆ haloalkyl, C₁-C₆ heteroalkyl, or cycloalkyl, wherein each alkyl, heteroalkyl, or cycloalkyl is optionally substituted with one or more R. In some embodiments, R^{a} is C₁-C₆ alkyl optionally substituted with one or more R. In some embodiments, R^{a} is C₁-C₆ alkyl. In some embodiments, R^{a} is CH₃.

In some embodiments, R^{a} is C₁-C₆ haloalkyl. In some embodiments, R^{a} is CHF₂. In some embodiments, R^{a} is C₁-C₆ heteroalkyl. In some embodiments, R^{a} is cycloalkyl optionally substituted with one or more R. In some embodiments, R^{a} is cycloalkyl.

In some embodiments, R^{b} is hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is optionally substituted with one or more R. In some embodiments, R^{b} is hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl). In some embodiments, R^{b} is C₁-C₆ alkyl, C₁-C₆ haloalkyl, C₁-C₆ heteroalkyl, or cycloalkyl, wherein each alkyl, heteroalkyl, or cycloalkyl is optionally substituted with one or more R. In some embodiments, R^{b} is C₁-C₆ alkyl optionally substituted with one or more R. In some embodiments, R^{b} is C₁-C₆ alkyl. In some embodiments, R^{b} is CH₃. In some embodiments, R^{b} is C₁-C₆ haloalkyl. In some embodiments, R^{b} is CHF₂. In some embodiments, R^{b} is C₁-C₆ heteroalkyl. In some embodiments, R^{b} is cycloalkyl optionally substituted with one or more R. In some embodiments, R^{b} is cycloalkyl.

In some embodiments, R^{c} and R^{d} are independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R. In some embodiments, R^{c} and R^{d} are independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl). In some embodiments, R^{c} and R^{d} are taken together with the atom to which they are attached to form a heterocycloalkyl optionally substituted with one or more R. In some embodiments, R^{c} and R^{d} are taken together with the atom to which they are attached to form a heterocycloalkyl.

In some embodiments, R^{c} is hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R. In some embodiments, R^{c} is hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl). In some embodiments, R^{c} is C₁-C₆ alkyl, C₁-C₆ haloalkyl, C₁-C₆ heteroalkyl, or cycloalkyl, wherein each alkyl, heteroalkyl, or cycloalkyl is optionally substituted with one or more R. In some embodiments, R^{c} is C₁-C₆ alkyl optionally substituted with one or more R. In some embodiments, R^{c} is C₁-C₆ alkyl. In some embodiments, R^{c} is CH₃. In some embodiments, R^{b} is C₁-C₆ haloalkyl. In some embodiments, R^{c} is CHF₂. In some embodiments, R^{c} is C₁-C₆ heteroalkyl. In some embodiments, R^{c} is cycloalkyl optionally substituted with one or more R. In some embodiments, R^{c} is cycloalkyl.

In some embodiments, R^{d} is hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R. In some embodiments, R^{d} is hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl). In some embodiments, R^{d} is C₁-C₆ alkyl, C₁-C₆ haloalkyl, C₁-C₆ heteroalkyl, or cycloalkyl, wherein each alkyl, heteroalkyl, or cycloalkyl is optionally substituted with one or more R. In some embodiments, R^{d} is C₁-C₆ alkyl optionally substituted with one or more R. In some embodiments, R^{d} is C₁-C₆ alkyl. In some embodiments, R^{d} is CH₃. In some embodiments, R^{b} is C₁-C₆ haloalkyl. In some embodiments, R^{d} is CHF₂. In some embodiments, R^{d} is C₁-C₆ heteroalkyl. In some embodiments, R^{d} is cycloalkyl optionally substituted with one or more R. In some embodiments, R^{d} is cycloalkyl.

In some embodiments, R is halogen, —CN, —OH, —SF₅, —SH, —S(═O)C₁-C₃ alkyl, —S(═O)₂C₁-C₃ alkyl, —S(═O)₂NH₂, —S(═O)₂NHC₁—C₃ alkyl, —S(═O)₂N(C₁-C₃ alkyl)₂, —S(═O)(═NC₁—C₃ alkyl)(C₁-C₃ alkyl), —NH₂, —NHC₁—C₃ alkyl, —N(C₁-C₃ alkyl)₂, —N═S(═O)(C₁-C₃ alkyl)₂, —C(═O)C₁-C₃ alkyl, —C(═O)OH, —C(═O)OC₁—C₃ alkyl, —C(═O)NH₂, —C(═O)NHC₁—C₃ alkyl, —C(═O)N(C₁-C₃ alkyl)₂, —P(═O)(C₁-C₃ alkyl)₂, C₁-C₃ alkyl, C₁-C₃alkoxy, C₁-C₃ haloalkyl, C₁-C₃ haloalkoxy, C₁-C₃ hydroxyalkyl, C₁-C₃ aminoalkyl, C₁-C₃ heteroalkyl, or C₃-C₆cycloalkyl. In some embodiments, two R on the same atom form an oxo. In some embodiments, R is halogen. In some embodiments, R is F. In some embodiments, R is —CN. In some embodiments, R is —OH. In some embodiments, R is —SF₅. In some embodiments, R is —SH. In some embodiments, R is —S(═O)C₁-C₃ alkyl. In some embodiments, R is —S(═O)₂C₁-C₃ alkyl. In some embodiments, R is —S(═O)₂NH₂. In some embodiments, R is —S(═O)₂NHC₁—C₃ alkyl. In some embodiments, R is —S(═O)₂N(C₁-C₃ alkyl)₂. In some embodiments, R is —S(═O)(═NC₁—C₃ alkyl)(C₁-C₃ alkyl). In some embodiments, R is —NH₂. In some embodiments, R is —NHC₁—C₃ alkyl. In some embodiments, R is —N(C₁-C₃ alkyl)₂. In some embodiments, R is —N═S(═O)(C₁-C₃ alkyl)₂. In some embodiments, R is —C(═O)C₁-C₃ alkyl. In some embodiments, R is —C(═O)OH. In some embodiments, R is —C(═O)OC₁—C₃ alkyl. In some embodiments, R is —C(═O)NH₂. In some embodiments, R is —C(═O)NHC₁—C₃ alkyl. In some embodiments, R is —C(═O)N(C₁-C₃ alkyl)₂. In some embodiments, R is —P(═O)(C₁-C₃ alkyl)₂. In some embodiments, R is C₁-C₃ alkyl. In some embodiments, R is C₁-C₃alkoxy. In some embodiments, R is C₁-C₃ haloalkyl. In some embodiments, R is C₁-C₃ haloalkoxy. In some embodiments, R is C₁-C₃ hydroxyalkyl. In some embodiments, R is C₁-C₃ aminoalkyl. In some embodiments, R is C₁-C₃ heteroalkyl. In some embodiments, R is or C₃-C₆cycloalkyl.

Non-limiting examples of compounds described herein, are compounds presented in Table 1, and pharmaceutically acceptable salts thereof. In some embodiments, the compound is selected from Table 1.

TABLE 1
Exemplary Compounds of the Disclosure

| Compound | | |
|---|---|---|
| No. | Structure | IUPAC |
| 1 | 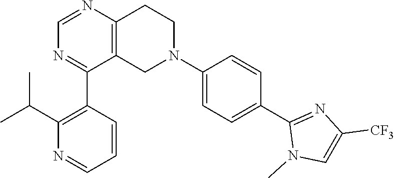 [SMILES: CC(C)c1ncccc1-c1ncnc2c1CN(c1ccc(-c3nc(C(F)(F)F)cn3C)cc1)CC2] | 4-(2-isopropylpyridin-3-yl)-6- (4-(1-methyl-4- (trifluoromethyl)-1H-imidazol- 2-yl)phenyl)-5,6,7,8- tetrahydropyrido[4,3- d]pyrimidine |
| 2 | 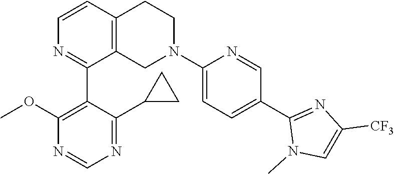 [SMILES: COc1ncnc(C2CC2)c1-c1nccc2c1CN(c1ccc(-c3nc(C(F)(F)F)cn3C)cn1)CC2] | 8-(4-cyclopropyl-6- methoxypyrimidin-5-yl)-2-(5- (1-methyl-4-(trifluoromethyl)- 1H-imidazol-2-yl)pyridin-2-yl)- 1,2,3,4-tetrahydro-2,7- naphthyridine |
| 3 | 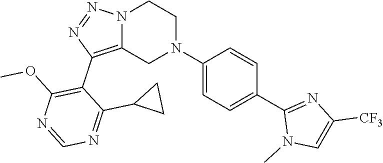 [SMILES: COc1ncnc(C2CC2)c1-c1nnn2c1CN(c1ccc(-c3nc(C(F)(F)F)cn3C)cc1)CC2] | 3-(4-cyclopropyl-6- methoxypyrimidin-5-yl)-5-(4- (1-methyl-4-(trifluoromethyl)- 1H-imidazol-2-yl)phenyl)- 4,5,6,7-tetrahydro- [1,2,3]triazolo[1,5-a]pyrazine |
| 4 | 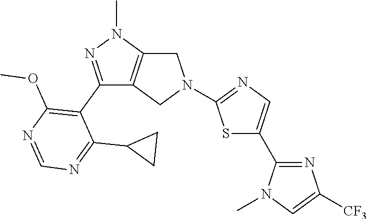 [SMILES: COc1ncnc(C2CC2)c1-c1nn(C)c2c1CN(c1ncc(-c3nc(C(F)(F)F)cn3C)s1)C2] | 2-(3-(4-cyclopropyl-6- methoxypyrimidin-5-yl)-1- methyl-4,6-dihydropyrrolo[3,4- c]pyrazol-5(1H)-yl)-5-(1- methyl-4-(trifluoromethyl)-1H- imidazol-2-yl)thiazole |
| 5 | 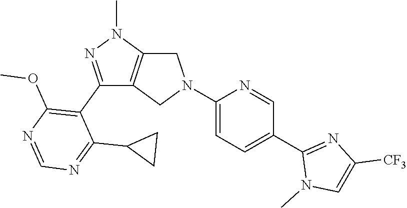 [SMILES: COc1ncnc(C2CC2)c1-c1nn(C)c2c1CN(c1ccc(-c3nc(C(F)(F)F)cn3C)cn1)C2] | 3-(4-cyclopropyl-6- methoxypyrimidin-5-yl)-1- methyl-5-(5-(1-methyl-4- (trifluoromethyl)-1H-imidazol- 2-yl)pyridin-2-yl)-1,4,5,6- tetrahydropyrrolo[3,4- c]pyrazole |
| 6 | 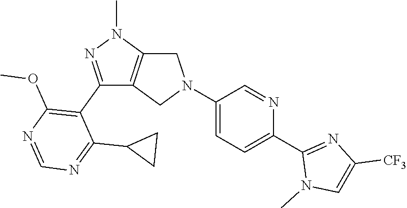 [SMILES: COc1ncnc(C2CC2)c1-c1nn(C)c2c1CN(c1ccc(-c3nc(C(F)(F)F)cn3C)nc1)C2] | 3-(4-cyclopropyl-6- methoxypyrimidin-5-yl)-1- methyl-5-(6-(1-methyl-4- (trifluoromethyl)-1H-imidazol- 2-yl)pyridin-3-yl)-1,4,5,6- tetrahydropyrrolo[3,4- clpyrazole |
| 7 | 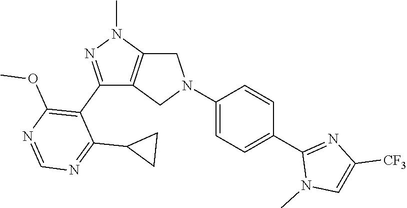 [SMILES: COc1ncnc(C2CC2)c1-c1nn(C)c2c1CN(c1ccc(-c3nc(C(F)(F)F)cn3C)cc1)C2] | 3-(4-cyclopropyl-6- methoxypyrimidin-5-yl)-1- methyl-5-(4-(1-methyl-4- (trifluoromethyl)-1H-imidazol- 2-yl)phenyl)-1,4,5,6- tetrahydropyrrolo[3,4- c]pyrazole |
| 8 | 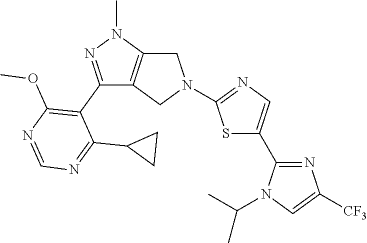 [SMILES: COc1ncnc(C2CC2)c1-c1nn(C)c2c1CN(c1ncc(-c3nc(C(F)(F)F)cn3C(C)C)s1)C2] | 2-(3-(4-cyclopropyl-6- methoxypyrimidin-5-yl)-1- methyl-4,6-dihydropyrrolo[3,4- c]pyrazol-5(1H)-yl)-5-(1- isopropyl-4-(trifluoromethyl)- 1H-imidazol-2-yl)thiazole |
| 9 | 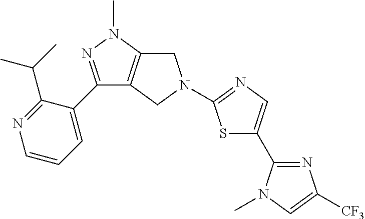 [SMILES: CC(C)c1ncccc1-c1nn(C)c2c1CN(c1ncc(-c3nc(C(F)(F)F)cn3C)s1)C2] | 2-(3-(2-isopropylpyridin-3-yl)- 1-methyl-4,6- dihydropyrrolo[3,4-c]pyrazol- 5(1H)-yl)-5-(1-methyl-4- (trifluoromethyl)-1H-imidazol- 2-yl)thiazole |
| 10 | 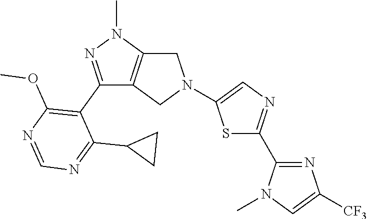 [SMILES: COc1ncnc(C2CC2)c1-c1nn(C)c2c1CN(c1cnc(-c3nc(C(F)(F)F)cn3C)s1)C2] | 5-(3-(4-cyclopropyl-6- methoxypyrimidin-5-yl)-1- methyl-4,6-dihydropyrrolo[3,4- c]pyrazol-5(1H)-yl)-2-(1- methyl-4-(trifluoromethyl)-1H- imidazol-2-yl)thiazole |
| 11 | 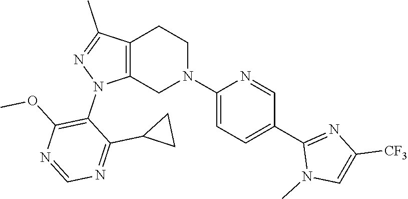 [SMILES: COc1ncnc(C2CC2)c1-n1nc(C)c2c1CN(c1ccc(-c3nc(C(F)(F)F)cn3C)cn1)CC2] | 1-(4-cyclopropyl-6- methoxypyrimidin-5-yl)-3- methyl-6-(5-(1-methyl-4- (trifluoromethyl)-1H-imidazol- 2-yl)pyridin-2-yl)-4,5,6,7- tetrahydro-1H-pyrazolo[3,4- c]pyridine |
| 12 | 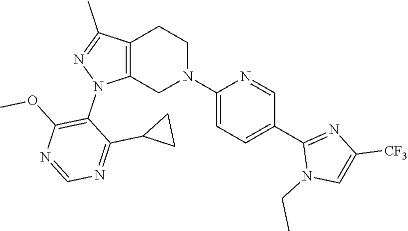 [SMILES: CCn1cc(C(F)(F)F)nc1-c1ccc(N2CCc3c(C)nn(-c4c(OC)ncnc4C4CC4)c3C2)nc1] | 1-(4-cyclopropyl-6- methoxypyrimidin-5-yl)-6-(5- (1-ethyl-4-(trifluoromethyl)-1H- imidazol-2-yl)pyridin-2-yl)-3- methyl-4,5,6,7-tetrahydro-1H- pyrazolo[3,4-c]pyridine |
| 13 | 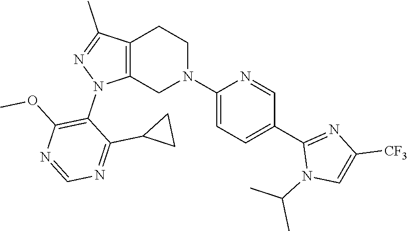 [SMILES: COc1ncnc(C2CC2)c1-n1nc(C)c2c1CN(c1ccc(-c3nc(C(F)(F)F)cn3C(C)C)cn1)CC2] | 1-(4-cyclopropyl-6- methoxypyrimidin-5-yl)-6-(5- (1-isopropyl-4- (trifluoromethyl)-1H-imidazol- 2-yl)pyridin-2-yl)-3-methyl- 4,5,6,7-tetrahydro-1H- pyrazolo[3,4-c]pyridine |
| 14 | 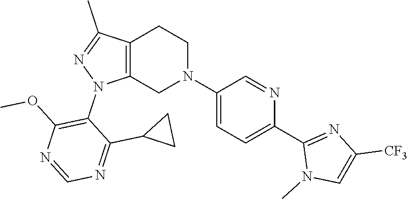 [SMILES: COc1ncnc(C2CC2)c1-n1nc(C)c2c1CN(c1ccc(-c3nc(C(F)(F)F)cn3C)nc1)CC2] | 1-(4-cyclopropyl-6- methoxypyrimidin-5-yl)-3- methyl-6-(6-(1-methyl-4- (trifluoromethyl)-1H-imidazol- 2-yl)pyridin-3-yl)-4,5,6,7- tetrahydro-1H-pyrazolo [3,4- c]pyridine |
| 15 | 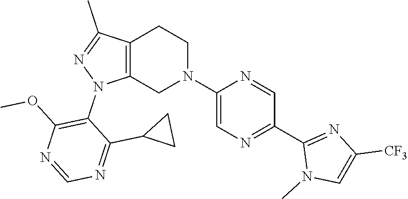 [SMILES: COc1ncnc(C2CC2)c1-n1nc(C)c2c1CN(c1cnc(-c3nc(C(F)(F)F)cn3C)cn1)CC2] | 1-(4-cyclopropyl-6- methoxypyrimidin-5-yl)-3- methyl-6-(5-(1-methyl-4- (trifluoromethyl)-1H-imidazol- 2-yl)pyrazin-2-yl)-4,5,6,7- tetrahydro-1H-pyrazolo[3,4- c]pyridine |
| 16 | 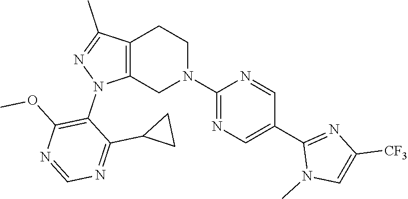 [SMILES: COc1ncnc(C2CC2)c1-n1nc(C)c2c1CN(c1ncc(-c3nc(C(F)(F)F)cn3C)cn1)CC2] | 1-(4-cyclopropyl-6- methoxypyrimidin-5-yl)-3- methyl-6-(5-(1-methyl-4- (trifluoromethyl)-1 H-imidazol- 2-yl)pyrimidin-2-yl)-4,5,6,7- tetrahydro-1H-pyrazolo[3,4- c]pyridine |
| 17 | 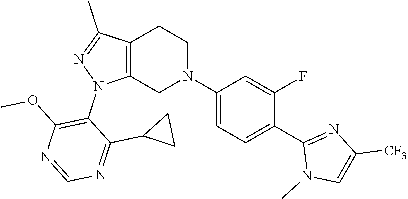 [SMILES: COc1ncnc(C2CC2)c1-n1nc(C)c2c1CN(c1ccc(-c3nc(C(F)(F)F)cn3C)c(F)c1)CC2] | 1-(4-cyclopropyl-6- methoxypyrimidin-5-yl)-6-(3- fluoro-4-(1-methyl-4- (trifluoromethyl)-1H-imidazol- 2-yl)phenyl)-3-methyl-4,5,6,7- tetrahydro-1H-pyrazolo[3,4- c]pyridine |
| 18 | 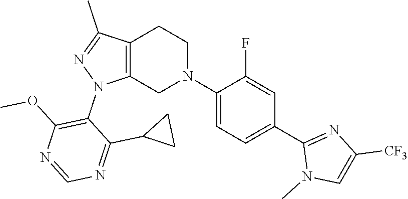 [SMILES: COc1ncnc(C2CC2)c1-n1nc(C)c2c1CN(c1ccc(-c3nc(C(F)(F)F)cn3C)cc1F)CC2] | 1-(4-cyclopropyl-6- methoxypyrimidin-5-yl)-6-(2- fluoro-4-(1-methyl-4- (trifluoromethyl)-1H-imidazol- 2-yl)phenyl)-3-methyl-4,5,6,7- tetrahydro-1H-pyrazolo[3,4- c]pyridine |
| 19 | 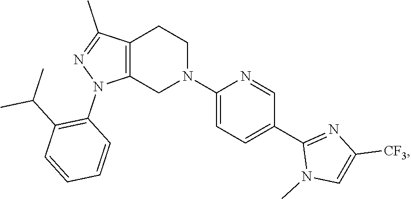 [SMILES: Cc1nn(-c2ccccc2C(C)C)c2c1CCN(c1ccc(-c3nc(C(F)(F)F)cn3C)cn1)C2] | 1-(2-isopropylphenyl)-3-methyl- 6-(5-(1-methyl-4- (trifluoromethyl)-1 H-imidazol- 2-yl)pyridin-2-yl)-4,5,6,7- tetrahydro-1H-pyrazolo[3,4- c]pyridine |
| 20 | 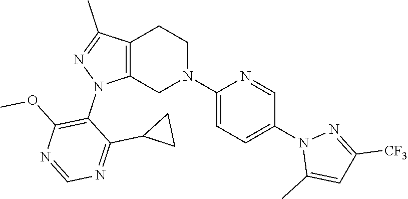 [SMILES: COc1ncnc(C2CC2)c1-n1nc(C)c2c1CN(c1ccc(-n3nc(C(F)(F)F)cc3C)cn1)CC2] | 1-(4-cyclopropyl-6- methoxypyrimidin-5-yl)-3- methyl-6-(5-(5-methyl-3- (trifluoromethyl)-1H-pyrazol-1- yl)pyridin-2-yl)-4,5,6,7- tetrahydro-1H-pyrazolo [3,4- c]pyridine |
| 21 | 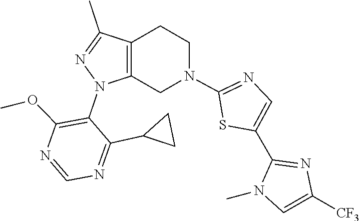 [SMILES: COc1ncnc(C2CC2)c1-n1nc(C)c2c1CN(c1ncc(-c3nc(C(F)(F)F)cn3C)s1)CC2] | 2-(1-(4-cyclopropyl-6- methoxypyrimidin-5-yl)-3- methyl-1,4,5,7-tetrahydro-6H- pyrazolo[3,4-c]pyridin-6-yl)-5- (1-methyl-4-(trifluoromethyl)- 1H-imidazol-2-yl)thiazole |
| 22 | 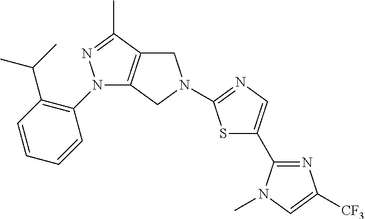 [SMILES: Cc1nn(-c2ccccc2C(C)C)c2c1CN(c1ncc(-c3nc(C(F)(F)F)cn3C)s1)C2] | 2-(1-(2-isopropylphenyl)-3- methyl-4,6-dihydropyrrolo[3,4- c]pyrazol-5(1H)-yl)-5-(1- methyl-4-(trifluoromethyl)-1H- imidazol-2-yl)thiazole |
| 23 | 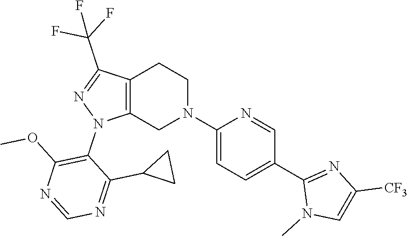 [SMILES: COc1ncnc(C2CC2)c1-n1nc(C(F)(F)F)c2c1CN(c1ccc(-c3nc(C(F)(F)F)cn3C)cn1)CC2] | 1-(4-cyclopropyl-6- methoxypyrimidin-5-yl)-6-(5- (1-methyl-4-(trifluoromethyl)- 1H-imidazol-2-yl)pyridin-2-yl)- 3-(trifluoromethyl)-4,5,6,7- tetrahydro-1H-pyrazolo[3,4- c]pyridine |
| 24 | 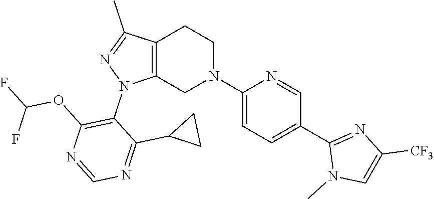 [SMILES: Cc1nn(-c2c(OC(F)F)ncnc2C2CC2)c2c1CCN(c1ccc(-c3nc(C(F)(F)F)cn3C)cn1)C2] | 1-(4-cyclopropyl-6- (difluoromethoxy)pyrimidin-5- yl)-3-methyl-6-(5-(1-methyl-4- (trifluoromethyl)-1H-imidazol- 2-yl)pyridin-2-yl)-4,5,6,7- tetrahydro-1H-pyrazolo[3,4- c]pyridine |
| 25 | 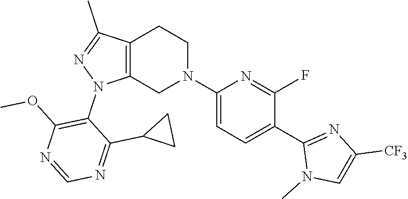 [SMILES: COc1ncnc(C2CC2)c1-n1nc(C)c2c1CN(c1ccc(-c3nc(C(F)(F)F)cn3C)c(F)n1)CC2] | 1-(4-cyclopropyl-6- methoxypyrimidin-5-yl)-6-(6- fluoro-5-(1-methyl-4- (trifluoromethyl)-1H-imidazol- 2-yl)pyridin-2-yl)-3-methyl- 4,5,6,7-tetrahydro-1H- pyrazolo[3,4-c]pyridine |
| 26 | 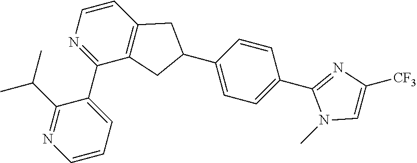 [SMILES: CC(C)c1ncccc1-c1nccc2c1CC(c1ccc(-c3nc(C(F)(F)F)cn3C)cc1)C2] | 1-(2-isopropylpyridin-3-yl)-6- (4-(1-methyl-4- (trifluoromethyl)-1H-imidazol- 2-yl)phenyl)-6,7-dihydro-5H- cyclopenta[c]pyridine |
| 27 | 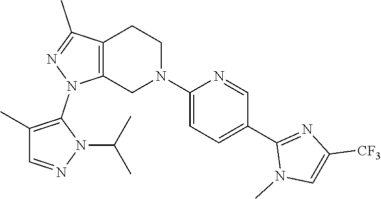 [SMILES: Cc1cnn(C(C)C)c1-n1nc(C)c2c1CN(c1ccc(-c3nc(C(F)(F)F)cn3C)cn1)CC2] | 1-(1-isopropyl-4-methyl-1H- pyrazol-5-yl)-3-methyl-6-(5-(1- methyl-4-(trifluoromethyl)-1H- imidazol-2-yl)pyridin-2-yl)- 4,5,6,7-tetrahydro-1H- pyrazolo[3,4-c]pyridine |
| 28 | 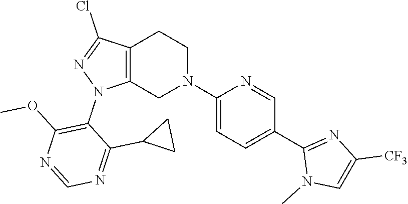 [SMILES: COc1ncnc(C2CC2)c1-n1nc(Cl)c2c1CN(c1ccc(-c3nc(C(F)(F)F)cn3C)cn1)CC2] | 3-chloro-1-(4-cyclopropyl-6- methoxypyrimidin-5-yl)-6-(5- (1-methyl-4-(trifluoromethyl)- 1H-imidazol-2-yl)pyridin-2-yl)- 4,5,6,7-tetrahydro-1H- pyrazolo[3,4-c]pyridine |
| 29 | 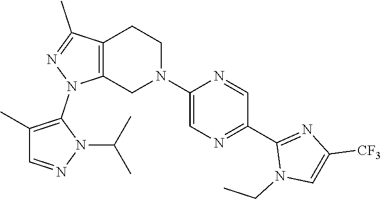 [SMILES: CCn1cc(C(F)(F)F)nc1-c1cnc(N2CCc3c(C)nn(-c4c(C)cnn4C(C)C)c3C2)cn1] | 6-(5-(1-ethyl-4- (trifluoromethyl)-1H-imidazol- 2-yl)pyrazin-2-yl)-1-(1- isopropyl-4-methyl-1H-pyrazol- 5-yl)-3-methyl-4,5,6,7- tetrahydro-1H-pyrazolo[3,4- c]pyridine |
| 30 | 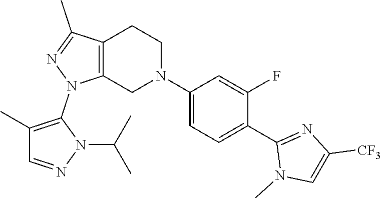 [SMILES: Cc1cnn(C(C)C)c1-n1nc(C)c2c1CN(c1ccc(-c3nc(C(F)(F)F)cn3C)c(F)c1)CC2] | 6-(3-fluoro-4-(1-methyl-4- (trifluoromethyl)-1H-imidazol- 2-yl)phenyl)-1-(1-isopropyl-4- methyl-1H-pyrazol-5-yl)-3- methyl-4,5,6,7-tetrahydro-1H- pyrazolo[3,4-c]pyridine |
| 31 | 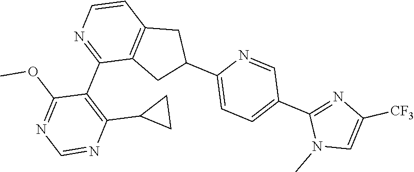 [SMILES: COc1ncnc(C2CC2)c1-c1nccc2c1CC(c1ccc(-c3nc(C(F)(F)F)cn3C)cn1)C2] | 1-(4-cyclopropyl-6- methoxypyrimidin-5-yl)-6-(5- (1-methyl-4-(trifluoromethyl)- 1H-imidazol-2-yl)pyridin-2-yl)- 6,7-dihydro-5H- cyclopenta[c]pyridine |
| 32 | 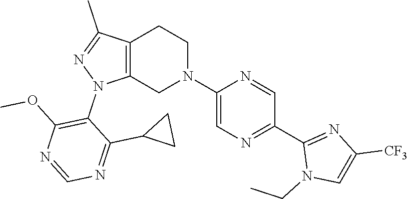 [SMILES: CCn1cc(C(F)(F)F)nc1-c1cnc(N2CCc3c(C)nn(-c4c(OC)ncnc4C4CC4)c3C2)cn1] | 1-(4-cyclopropyl-6- methoxypyrimidin-5-yl)-6-(5- (1-ethyl-4-(trifluoromethyl)-1H- imidazol-2-yl)pyrazin-2-yl)-3- methyl-4,5,6,7-tetrahydro-1H- pyrazolo[3,4-c]pyridine |

Additional non-limiting examples of compounds described herein, are compounds presented in Table 2, and pharmaceutically acceptable salts thereof. In some embodiments, the compound is selected from Table 2.

TABLE 2
Exemplary Compounds of the Disclosure

| Compound | | |
|---|---|---|
| No. | Structure | IUPAC |
| 33 | 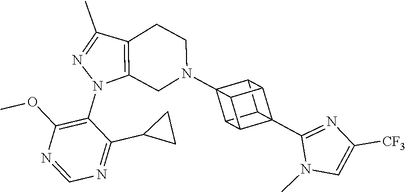 [SMILES: COc1ncnc(C2CC2)c1-n1nc(C)c2c1CN(C13C4C5C1C1C3C4C51c1nc(C(F)(F)F)cn1C)CC2] | 1-(4-cyclopropyl-6- methoxypyrimidin-5-yl)- 3-methyl-6-(4-(1-methyl- 4-(trifluoromethyl)-1H- imidazol-2-yl)cuban-1- yl)-4,5,6,7-tetrahydro-1H- pyrazolo[3,4-c]pyridine |

Table 3 presents corresponding biological data for USP1 IC50 (nM) and MDA-MB-436 IC50 (nM) for the compounds presented in Table 1.

TABLE 3

| | USP1 IC₅₀ | MDA-MB-436 IC₅₀ |
|---|---|---|
| Compound No. | (nM) | (nM) |
| 1 | C | C |
| 2 | B | B |
| 3 | B | B |
| 4 | B | B |
| 5 | C | |
| 6 | C | |
| 7 | B | |
| 8 | B | B |
| 9 | B | |
| 10 | B | B |
| 11 | B | A |
| 12 | B | A |
| 13 | B | B |
| 14 | B | B |
| 15 | A | A |
| 16 | B | B |
| 17 | A | A |
| 18 | B | A |
| 19 | A | A |
| 20 | B | B |
| 21 | B | B |
| 22 | B | C |
| 23 | B | B |
| 24 | B | B |
| 25 | A | A |
| 26 | A | B |
| 27 | A | A |
| 28 | A | A |
| 29 | A | A |
| 30 | A | A |
| 31 | B | C |
| 32 | A | B |

IC₅₀ (nM): 0 < A ≤ 50; 50 < B ≤ 1,000; 1,000 < C ≤ 10,000

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Isotopically Enriched Compounds

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford some therapeutic advantages, such as increased in vivo half-life and/or exposure, or may provide a compound useful for investigating in vivo routes of drug elimination and metabolism.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹³¹I, and ¹²⁵I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar.

In some embodiments, the compound is deuterated in at least one position. In some embodiments, the compounds disclosed herein have some or all of the ¹H atoms replaced with ²H atoms.

The methods of synthesis for deuterium-containing compounds are known in the art and include, by way of non-limiting example only, the procedure described in U.S. Pat. Nos. 5,846,514 and 6,334,997, and the following synthetic methods. For example, deuterium substituted compounds may be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

### C. Methods of Treatment

Ubiquitin Specific Protease 1 (USP1) is a member of the ubiquitin-specific processing family of proteases. USP1 is a deubiquitinating enzyme (“DUB”) and deubiquitinates its substrates involved in key oncogenic pathways to modulate their functions. Among its roles, USP1 can exhibit DNA-mediated activation at the replication fork, protects the fork, and promote survival in BRCA1-deficient cells. As loss of both USP1 and BRCA1 leads to replication fork degradation, inhibition of USP1 can selectively decrease the viability, or kill, tumor cells with defects in BRCA defects without affecting the survival of cells with normal BRCA function.

In the United States (US), it has been estimated that inherited BRCA1 and BRCA2 mutations are present in 5-10% of breast cancers and 10-15% of ovarian cancers. Breast cancer is the most common cancer in the world and the most common malignancy in women. BRCA1 and BRCA2 can be detected in at least 5% of unselected breast cancer patients and in approximately 30% of patients with a family history of developing breast or ovarian cancer. At present, treatment options including chemotherapy and immune checkpoint inhibitors are limited for breast cancer patients with germline BRCA mutations, more aggressive progression and higher risk of recurrence. While PARP inhibitors have been approved by the US Food and Drug Administration (FDA) as monotherapies for deleterious/suspected deleterious germline BRCA-mutated, HER2-negative breast cancer, in some cases, resistance to the PARP inhibitors can be observed to develop quickly in breast cancer patients. Ovarian cancers represent a heterogenous group of solid tumors. On average, one in five ovarian cancer can be associated with germline mutations. Of those ovarian cancers with germline mutations, 65-85% can be associated with germline BRCA mutations. Similar to the breast cancer setting, while the PARP inhibitors can be the first-line maintenance therapy for patients with BRCA-mutated ovarian cancer, those patients can develop resistance to the PARP inhibitors.

The compounds described herein can be used as inhibitors of USP1. Such compounds can exhibit BRCA1 and/or BRCA2 mutant-selective, anti-proliferative activities. The compounds described herein can be used to treat BRCA1 and/or BRCA2 mutant or homologous recombination (HRD) positive cancers. The compounds described herein can exhibit anti-proliferative activities in cancer cells with a BRCA1 and/or BRCA2 mutation, particularly MDA-MB-436 cells. The compounds described herein may not exhibit similar anti-proliferative activities in cancer cells with wild-type BRCA, particularly SNG-M cells. In some embodiments, the compounds described herein can show selectivity for mutant BRCA1 and/or BRCA2 over wild-type BRCA of at least 50-fold, 100-fold, 150-fold, 200-fold, 250-fold, 300-fold, 350-fold, 400-fold, 450-fold, 500-fold, 550-fold, 600-fold, or more.

The compounds described herein can be used in the preparation of medicaments for the prevention or treatment of diseases or conditions. In some embodiments, the compounds described herein are used in a method of modulating USP1 in a subject. In some embodiments, the compounds described herein are used in a method of inhibiting USP1 in subject. In some embodiments, the compounds described herein are used in a method of inhibiting or reducing DNA repair activity modulated by USP1 in a subject. In some embodiments, the compounds herein are used in a method of treating a disease or disorder associated with USP1 in a subject. In some embodiments, the compounds described herein are used in a method of treating a disease or disorder associated with modulation of USP in a subject. In addition, a method for modulating, inhibiting, or treating any of the diseases or conditions described herein in a subject in need of such treatment, involves administration of pharmaceutical compositions containing at least one compound described herein or a pharmaceutically acceptable salt thereof, in therapeutically effective amounts to said subject.

In the case wherein the patient's condition does not improve, upon the doctor's discretion the administration of the compounds can be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose can conveniently be presented in a single dose or as divided doses administered simultaneously (or over a short period of time) or at appropriate intervals, for example as two, three, four or more sub-doses per day.

In one aspect, the disclosure provides a method of modulating USP1 in a subject, comprising administering to the subject a compound described herein or a pharmaceutically acceptable salt thereof.

In one aspect, the disclosure provides a method of inhibiting USP1 in a subject, comprising administering to the subject a compound described herein or a pharmaceutically acceptable salt thereof.

In one aspect, the disclosure provides a method of inhibiting or reducing DNA repair activity modulated by USP1 in a subject in need thereof, the method comprising administering a therapeutically effective amount of a compound described herein, or a pharmaceutically acceptable salt thereof, or a pharmaceutical composition described herein.

In one aspect, the disclosure provides a method of treating a disease or disorder associated with USP1 in a subject in need thereof, comprising administering to the subject a therapeutically effective amount of a compound described herein, or a pharmaceutically acceptable salt thereof, or a pharmaceutical composition of described herein. In some embodiments, the disease or a disorder is cancer.

In one aspect, the disclosure provides a method of treating a disease or disorder associated with modulation of USP1 in a subject, comprising administering to the subject a therapeutically effective amount of a compound described herein, or a pharmaceutically acceptable salt thereof, or a pharmaceutical composition of described herein. In some embodiments, the disease or disorder is cancer.

In one aspect, the disclosure provides a method of treating cancer in a subject, comprising administering to the subject in need thereof an effective amount of a compound described herein, or a pharmaceutically acceptable salt thereof, or a pharmaceutical composition of described herein.

In one aspect, the disclosure provides a method of treating cancer in a subject, comprising administering to the subject in need thereof an amount of a compound described herein, or a pharmaceutically acceptable salt thereof, or a pharmaceutical composition of described herein. In some embodiments, the cancer is leukemia, acute myeloid leukemia (AML), chronic myeloid leukemia, acute lymphoblastic leukemia (ALL), non-Hodgkin lymphoma (NHL), Hodgkin lymphoma (HL), or multiple myeloma (MM).

In some embodiments, the cancer is a carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer. In some embodiments, the cancer is a homolgous-recombination deficient cancer. In some embodiments, the cancer comprises cancer cells with a mutation in a gene encoding p53.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipidus; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or utterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

In one aspect, the disclosure provides a method of treating cancer in a subject, comprising administering to the subject in need thereof an effective amount of a compound described herein, or a pharmaceutically acceptable salt thereof, or a pharmaceutical composition of described herein. In some embodiments, the cancer can comprise cancer cells with elevated levels of RAD 18 mRNA expression. In some embodiments, elevated levels of RAD 18 are elevated levels of RAD 18 protein. In some embodiments, RAD 18 levels can be detected using quantitative methods like microarray, RNA-Seq, or reverse transcriptase polymerase chain reaction (RT-PCR). In some embodiments, the levels of RAD 18 in a cancer cell can be detected prior to administration of the compounds described herein. In some embodiments, RAD 18 levels can be detected in a cancer sample obtained from a subject. In some embodiments, if a subject has elevated levels of RAD 18, the subject can be treated with the compounds described herein. In some embodiments, elevated levels of RAD 18 in cancer cells indicate that a subject administered the compounds or pharmaceutical compositions described herein is responsive to treatment using the compounds or pharmaceutical compositions described herein. In some embodiments, the compounds described herein are not administered to a subject with elevated levels of RAD 18.

In some embodiments, the cancer is a DNA damage repair pathway deficient cancer. In some embodiments, the cancer is a PARP inhibitor resistant or refractory BRCA1 or BRCA2-mutant cancer. In some embodiments, the cancer comprises cells with elevated levels of RAD 18, where the elevated levels of RAD 18 are at least as high as the RAD 18 mRNA and/or protein levels in ES2 cells or HEP3B217 cells.

In some embodiments, the cancer is a BRCA1 mutant cancer and/or a BRCA2 mutant cancer. In some embodiments, the cancer is a BRCA1 or BRCA2 wildtype cancer. In some embodiments, the cancer is a BRCA1-deficient cancer. In some embodiments, the cancer is a BRCA2-deficient cancer. In some embodiments, the cancer that comprises cancer cells with a mutation in a gene that encodes BRCA1 and/or BRCA2. In some embodiments, the cancer is a BRCA1 mutant cancer and BRCA2 deficient cancer. In some embodiments, the cancer is a BRCA1 deficient cancer and BRCA2 mutant cancer. In some embodiments, the cancer comprises cells with elevated levels of RAD 18, where the elevated levels of RAD 18 are at least as high as the RAD 18 mRNA and/or protein levels in ES2 cells or HEP3B217 cells.

### EXAMPLES

The following examples are offered to illustrate, but not to limit the claimed disclosure. The following examples further illustrate the disclosure but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

The compounds and salts described herein can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in the synthesis schemes below, the steps in some cases can be performed in a different order than the order shown below. Numberings or R groups in each scheme do not necessarily correspond to that of the claims or other schemes or tables herein.

### ABBREVIATIONS

| | |
|---|---|
| ACN | Acetonitrile |
| Ar | Argon |
| BINAP | 2,2′-bis(diphenylphosphino)-1,1′-binaphthyl |
| DBU | 1,8-Diaza-7-bicyclo[5.4.0]undecene |
| DCE | 1,2-Dichloroethane |
| DCM | Dichloromethane |
| DIEA, DIPEA | diisopropylethylamine |
| DMF | N,N-Dimethylformamide |
| DMSO | Dimethylsulfoxide |
| EtOAc, EA | Ethyl Acetate |
| EtOH | Ethanol |
| hr(s) | hour(s) |
| IPA | Isopropyl Alcohol |
| LC-MS | Liquid chromatography-mass spectrometry |
| m-CPBA | meta-Chloroperoxybenzoic acid |
| MeOH | Methaol |
| NaOAc | Sodium acetate |
| NBS | N-Bromobutanimide |
| NIS | N-Iodosuccinimide |
| PE | Petroleum ether |
| Pre-TLC | Preparative-thin layer chromatography |
| RT, rt, r.t. | Room temperature |
| TEA | Triethylamine |
| TFA | Trifluoroacetic acid |
| THF | Tetrahydrofuran |

### Examples A Biological Assays

### Example A1: Enzymatic Assay

Human recombinant USP1/UAF1 expressed in baculovirus infected Sf21 cells were used (R & D, E-568-050). Test compound and/or vehicle was incubated with 2 nM of USP1/UAF1 in modified HEPES buffer pH 8.0 for 15 minutes at RT. The reaction was initiated by addition of 500 nM of Ubiquitin Rhodamine 110 (R & D, U-555-050) for kinetic reading. Slope change of fluorescence intensity was read spectrofluorimetrically at 485 nm/535 nm. Dose response of test compounds or reference compound ML-323 was analyzed by nonlinear regression of GraphPad prism software. Results of the assay are illustrated in Table 3.

### Example A2: MDA-MB-436 Breast Cancer Cell Culture

MDA-MB-436 cells were grown in Leibovitz's L-15 medium with 10 μg/ml insulin, 16 μg/ml glutathione, 10% FBS. Cells were passaged at subconfluence after trypsinization and maintained in incubators at 37° C. in a humidified atmosphere with 5% CO₂.

### Example A3: MDA-MB-436 Breast Cancer Cell Proliferation Assay

Cell proliferation was determined using CellTiter-Glo® Luminescent Cell Viability Assay (Promega, #G7573). MDA-MB-436 cells were seeded in 384-well plates and allowed to attach for 24 h. Compounds were added into 384-well plate by ECHO, and incubated at 37° C. in a humidified atmosphere with 5% CO₂. After 7 days, CellTiter-Glo® was added into 384 well plates, contents were mixed on an orbital shaker at 400 g for 2 min before centrifuging the plate for 2 min at 1000 rpm. After incubation at RT for 30 min, luminescence was read on envision. Results of the assay are illustrated in Table 3.

### Examples B Chemical Synthesis

### Example B: Synthesis of Intermediate C

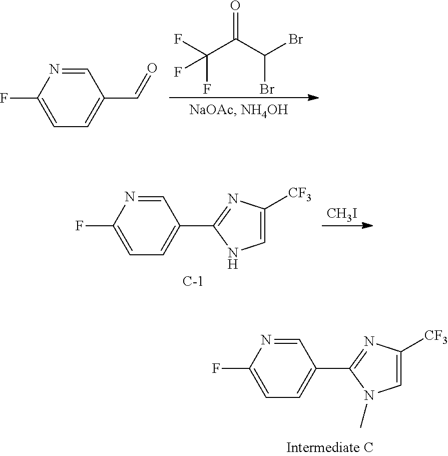

### 2-fluoro-5-(4-(trifluoromethyl)-1H-imidazol-2-yl)pyridine

To a solution of sodium acetate (13.11 g, 159.88 mmol) in water (100 mL) was added 3,3-dibromo-1,1,1-trifluoropropan-2-one (32.36 g, 119.91 mmol) dropwise at 0° C. The mixture was stirred at 100° C. for 1 hr. A solution of 6-fluoronicotinaldehyde (10.00 g, 79.94 mmol), MeOH (150 mL), and ammonium hydroxide (approximately 25-28 wt %) (150 mL) was added to the cooled mixture at 0° C. The mixture was stirred at room temperature for 16 hrs. the volatiles were removed in vacuo to give a residue. The residue was diluted with water (100 mL) and extracted with EtOAc (80 mL×5). The combined organic fractions were washed with brine (100 mL), dried over Na₂SO₄, filtered through a Celite® pad and the filtrate was concentrated under reduced pressure to give the title compound (18.0 g, 77.87 mmol, 97% yield) as a yellow solid, which was used for next step without further purification.

¹H NMR (400 MHz, DMSO-d₆) δ=8.80 (d, J=2.4 Hz, 1H), 8.60-8.43 (m, 1H), 8.03 (s, 1H), 7.41-7.28 (m, 1H).

### 2-fluoro-5-(1-methyl-4-(trifluoromethyl)-1H-imidazol-2-yl)pyridine

To a solution of 2-fluoro-5-(4-(trifluoromethyl)-1H-imidazol-2-yl)pyridine (5.00 g, 21.63 mmol) in DMF (50 mL) were added Cs₂CO₃ (14.09 g, 43.26 mmol) and CH₃I (4.61 g, 32.45 mmol) at 0° C. The mixture was stirred at room temperature for 16 hrs under Ar. To the reaction mixture was added water (60 mL) and the aqueous phase was extracted with EtOAc (40 mL×2). The combined organic layers were washed with brine (50 mL), dried over Na₂SO₄, filtered through a Celite® pad and the filtrate was concentrated under reduced pressure to give a residue, which was purified by flash column chromatography on silica gel eluting with ethyl acetate (from 0% to 10%) in petroleum ether to give the title compound (4.1 g, 16.72 mmol, 77% yield) as a white solid.

¹H NMR (400 MHz, DMSO-d₆) δ=8.63-8.59 (m, 1H), 8.45-8.26 (m, 1H), 8.05-8.00 (m, 1H), 7.45-7.28 (m, 1H), 3.82 (s, 3H).

### Example B2: Synthesis of Intermediate E

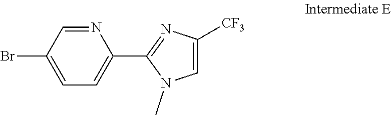 [SMILES: Cn1cc(C(F)(F)F)nc1-c1ccc(Br)cn1]

The synthetic route for Intermediate E was similar to that of Intermediate C by replacing 6-fluoronicotinaldehyde with 5-bromopicolinaldehyde. The crude product was purified by flash column chromatography on silica gel eluting with ethyl acetate (from 0% to 16%) in petroleum ether to give the title compound (300 mg, 0.98 mmol, 14% yield) as a yellow solid.

¹H NMR (400 MHz, CDCl₃) δ=8.65-8.50 (m, 1H), 8.14-8.01 (m, 1H), 7.85-7.75 (m, 1H), 7.22 (s, 1H), 4.05 (s, 3H).

### Example B3: Synthesis of Intermediate G

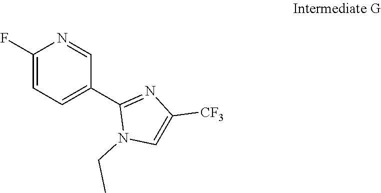 [SMILES: CCn1cc(C(F)(F)F)nc1-c1ccc(F)nc1]

The synthetic route for Intermediate G was similar to that of Intermediate C by replacing iodomethane with iodoethane. The crude product was purified by flash column chromatography on silica gel eluting with ethyl acetate (from 0% to 17%) in petroleum ether to give the title compound (1.90 g, 7.33 mmol, 56% yield) as a yellow oil.

LC-MS(ESI+): m/z 260.0 (M+H)⁺.

¹H NMR (400 MHz, CDCl₃) δ=8.49-8.41 (m, 1H), 8.18-8.02 (m, 1H), 7.50-7.38 (m, 1H), 7.15-7.05 (m, 1H), 4.15-4.03 (m, 2H), 1.49 (t, J=7.2 Hz, 3H).

### Example B4: Synthesis of Intermediate H

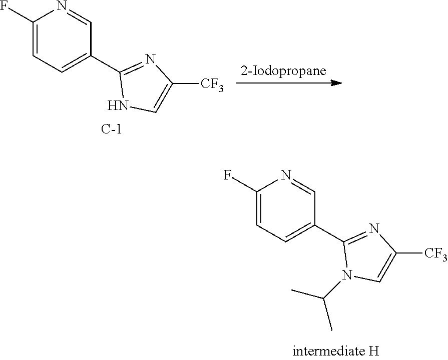

### 2-fluoro-5-(1-isopropyl-4-(trifluoromethyl)-1H-imidazol-2-yl)pyridine

To a solution of 2-fluoro-5-(4-(trifluoromethyl)-1H-imidazol-2-yl)pyridine (3.00 g, 12.98 mmol) and Cs₂CO₃ (12.70 g, 38.98 mmol) in DMF (50 mL) was added 2-iodopropane (11.05 g, 65.00 mmol). The mixture was stirred at 75° C. for 24 hrs. To the cooled reaction mixture was added water (50 mL) and extracted with EtOAc (50 mL×2). The combined organic layers were washed with brine (50 mL×3), dried over Na₂SO₄, filtered through a Celite® pad and the filtrate was concentrated under reduced pressure to give a residue, which was purified by flash column chromatography on silica gel eluting with ethyl acetate (from 0% to 10%) in petroleum ether to give the title compound (1.7 g, 6.22 mmol, 48% yield) as a yellow oil.

LC-MS(ESI+): m/z 274.1 (M+H)⁺.

¹H NMR (400 MHz, CDCl₃) δ=8.45-8.37 (m, 1H), 8.13-8.02 (m, 1H), 7.47 (s, 1H), 7.13-7.05 (m, 1H), 4.54-4.43 (m, 1H), 1.53-1.45 (m, 6H).

### Example B5: Synthesis of Intermediate I

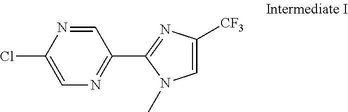 [SMILES: Cn1cc(C(F)(F)F)nc1-c1cnc(Cl)cn1]

The synthetic route for Intermediate I was similar to that of Intermediate C by replacing 6-fluoronicotinaldehyde with 5-chloropyrazine-2-carbaldehyde. The crude product was purified by flash column chromatography on silica gel eluting with ethyl acetate (from 0% to 5%) in petroleum ether to give the title compound (550.0 mg, 2.09 mmol, 74% yield) as a white solid.

LC-MS(ESI+): m/z 262.9 (M+H)⁺.

¹H NMR (400 MHz, CDCl₃) δ=9.21-9.13 (m, 1H), 8.51-8.43 (m, 1H), 7.29 (s, 1H), 4.05 (s, 3H).

### Example B6: Synthesis of Intermediate J

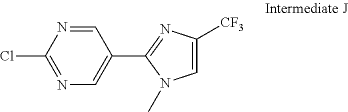 [SMILES: Cn1cc(C(F)(F)F)nc1-c1cnc(Cl)nc1]

The synthetic route for Intermediate J was similar to that of Intermediate C by replacing 6-fluoronicotinaldehyde with 2-chloropyrimidine-5-carbaldehyde. The crude product was purified by flash column chromatography on silica gel eluting with ethyl acetate (from 0% to 10%) in petroleum ether to give the title compound (420.0 mg, 1.60 mmol, 57% yield) as a yellow solid.

LC-MS(ESI+): m/z 263.0 (M+H)⁺.

¹H NMR (400 MHz, DMSO-d₆) δ=9.19 (s, 2H), 8.15 (s, 1H), 3.93 (s, 3H).

### Example B7: Synthesis of Intermediate K

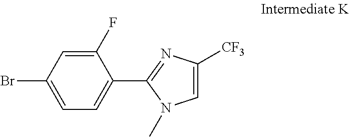 [SMILES: Cn1cc(C(F)(F)F)nc1-c1ccc(Br)cc1F]

The synthetic route for Intermediate K was similar to that of Intermediate C by replacing 6-fluoronicotinaldehyde with 4-bromo-2-fluorobenzaldehyde. The crude product was purified by flash column chromatography on silica gel eluting with ethyl acetate (from 0% to 5%) in petroleum ether to give the title compound (720.0 mg, 2.23 mmol, 76% yield) as a yellow solid.

LC-MS(ESI+): m/z 322.8 (M+H)⁺.

¹H NMR (400 MHz, DMSO-d₆) δ=8.07-8.00 (m, 1H), 7.86-7.74 (m, 1H), 7.68-7.52 (m, 2H), 3.61 (d, J=1.6 Hz, 3H).

### Example B8: Synthesis of Intermediate L

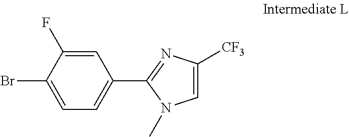 [SMILES: Cn1cc(C(F)(F)F)nc1-c1ccc(Br)c(F)c1]

The synthetic route for Intermediate L was similar to that of Intermediate C by replacing 6-fluoronicotinaldehyde with 4-bromo-3-fluorobenzaldehyde. The crude product was purified by flash column chromatography on silica gel eluting with ethyl acetate (from 0% to 10%) in petroleum ether to give the title compound (822 mg, 2.54 mmol, 78% yield) as a yellow solid.

LC-MS(ESI+): m/z 322.9 (M+H)⁺.

¹H NMR (400 MHz, DMSO-d₆) δ=7.99 (s, 1H), 8.15-8.05 (m, 1H), 7.80-7.71 (m, 1H), 7.58-7.51 (m, 1H), 3.82 (s, 3H).

### Example B9: Synthesis of Intermediate M

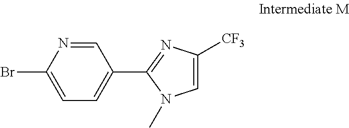 [SMILES: Cn1cc(C(F)(F)F)nc1-c1ccc(Br)nc1]

The synthetic route for Intermediate M was similar to that of Intermediate C by replacing 6-fluoronicotinaldehyde with 6-bromonicotinaldehyde. The crude product was purified by flash column chromatography on silica gel eluting with ethyl acetate (from 0% to 20%) in petroleum ether to give the title compound (2.91 g, 9.50 mmol, 55% yield) as a yellow solid.

LC-MS(ESI+): m/z 305.9 (M+H)⁺.

¹H NMR (400 MHz, DMSO-d₆) δ=8.74 (d, J=2.4 Hz, 1H), 8.13-8.10 (m, 1H), 8.04 (s, 1H), 7.81 (d, J=8.0 Hz, 1H), 3.83 (s, 3H).

### Example B10: Synthesis of Compound 1

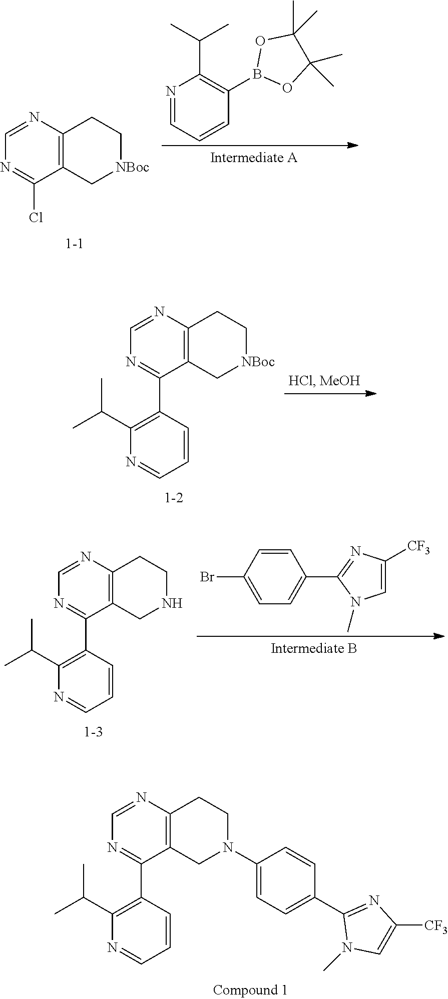

### Tert-butyl 4-(2-isopropylpyridin-3-yl)-7,8-dihydropyrido[4,3-d]pyrimidine-6(5H)-carboxylate

To a solution of tert-butyl 4-chloro-5H,6H,7H,8H-pyrido[4,3-d]pyrimidine-6-carboxylate (409.0 mg, 1.52 mmol) in dioxane (10 mL) and water (2 mL) were added 2-(propan-2-yl)-3-(tetramethyl-1,3,2-dioxaborolan-2-yl)pyridine (562.1 mg, 2.27 mmol), Pd(dppf)Cl₂ (111.0 mg, 0.15 mmol) and Na₂CO₃ (321.4 mg, 3.03 mmol) under N₂. The mixture was stirred at 100° C. for 16 hrs. To the cooled reaction mixture was added water (30 mL) and extracted with EtOAc (20 mL×2). The combined organic layers were washed with brine (30 mL), dried over Na₂SO₄, filtered through a Celite® pad and the filtrate was concentrated under reduced pressure to give a residue, which was purified by flash column chromatography on silica gel eluting with ethyl acetate (from 0% to 20%) in petroleum ether to give the title compound (400.0 mg, 1.13 mmol, 74% yield) as yellow oil.

LC-MS(ESI+): m/z 355.2 (M+H)⁺.

### 4-(2-isopropylpyridin-3-yl)-5,6,7,8-tetrahydropyrido[4,3-d]pyrimidine

A mixture of tert-butyl 4-[2-(propan-2-yl)pyridin-3-yl]-5H,6H,7H,8H-pyrido[4,3-d]pyrimidine-6-carboxylate (400.0 mg, 1.13 mmol) in HCl/MeOH (3 mol/L, 3 mL) was stirred at room temperature for 6 hrs. The mixture was concentrated in vacuo to give a residue. Then, EtOAc (20 mL) and water (10 mL) were added into the residue. The aqueous phase was basified with sat. NaHCO₃ solution and then extracted with EtOAc (15 mL×3). The combined organic layers were washed with brine (30 mL), dried over Na₂SO₄, filtered through a Celite® pad and the filtrate was concentrated under reduced pressure to give the crude product, which was purified by reverse column chromatography (H₂O/ACN=100/0 to 9/1) to give the title compound (200.0 mg, 0.79 mmol, 70% yield) as a yellow solid.

LC-MS(ESI+): m/z 255.1 (M+H)⁺.

### 4-(2-isopropylpyridin-3-yl)-6-(4-(1-methyl-4-(trifluoromethyl)-1H-imidazol-2-yl)phenyl)-5,6,7,8-tetrahydropyrido[4,3-d]pyrimidine

To a solution of 2-(propan-2-yl)-3-{5H,6H,7H,8H-pyrido[4,3-d]pyrimidin-4-yl}pyridine (50.0 mg, 0.20 mmol) in toluene (5 mL) were added 2-(4-bromophenyl)-1-methyl-4-(trifluoromethyl)-1H-imidazole (50.0 mg, 0.16 mmol), sodium tert-butoxide (15.7 mg, 0.16 mmol) and Pd-PEPPSI™-IPent catalyst (0.83 mg, 0.001 mmol) under N₂. The mixture was stirred at 100° C. for 16 hrs. To the cooled reaction mixture was added water (10 mL) and extracted with EtOAc (10 mL×2). The combined organic layers were washed with brine (10 mL), dried over Na₂SO₄, filtered through a Celite® pad and the filtrate was concentrated under reduced pressure to give a residue, which was purified by pre-TLC (PE/EA=1/3) to give the title compound.

LC-MS(ESI+): m/z 479.2 (M+H)⁺.

¹H NMR (400 MHz, DMSO-d₆) δ=9.09 (s, 1H) −8.74-8.73 (m, 1H), 7.84 (s, 1H), 7-80-7.76 (m, 1H), 7.55 (d, J=8.8 Hz, 2H) −7.43-7.40 (m, 1H), 6.93 (d, J=9.2 Hz, 2H)-4.24-4.05 (m, 2H) −3.95-3.75 (m, 2H), 3.72 (s, 3H) −3.23-3.14 (m, 2H) −2.79-2.60 (m, 1H), 1.16 (d, J=6.8 Hz, 6H).

### Example B11: Synthesis of Compound 2

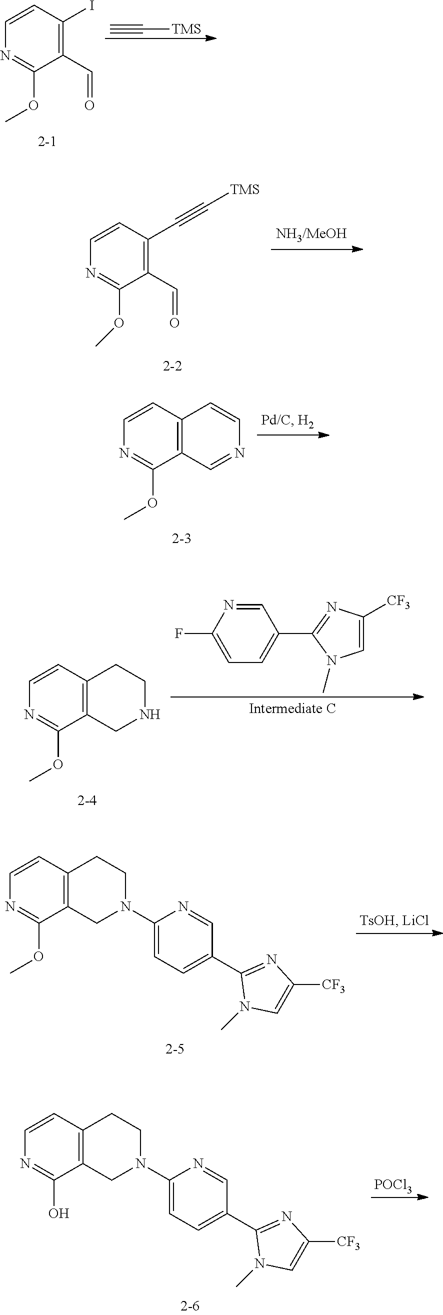

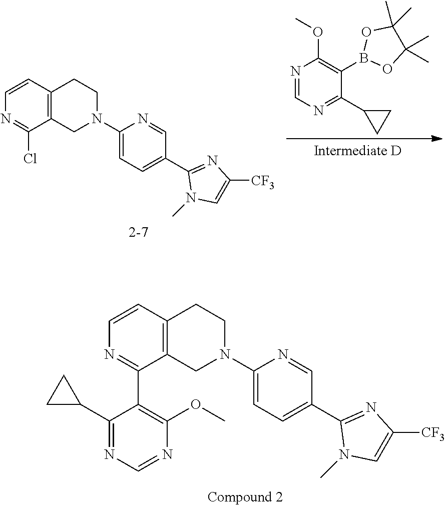 [SMILES: Cn1cc(C(F)(F)F)nc1-c1ccc(N2CCc3ccnc(Cl)c3C2)nc1 ; COc1ncnc(C2CC2)c1B1OC(C)(C)C(C)(C)O1 ; COc1ncnc(C2CC2)c1-c1nccc2c1CN(c1ccc(-c3nc(C(F)(F)F)cn3C)cn1)CC2]

### 2-methoxy-4-((trimethylsilyl)ethynyl)nicotinaldehyde

To a solution of 4-iodo-2-methoxypyridine-3-carbaldehyde (1.00 g, 3.80 mmol) and Trimethylsilylacetylene (747.0 mg, 7.61 mmol) in THF (10 mL) were added TEA (1.15 g, 11.41 mmol), CuI (72.0 mg, 0.38 mmol) and bis(triphenylphosphine)palladium(II) chloride (267.0 mg, 0.38 mmol). The mixture was degassed and refilled with N₂ for three times, then the reaction mixture was stirred at 60° C. for 3 hrs. The mixture was concentrated in vacuo to give a residue, which was purified by flash column chromatography on silica gel eluting with ethyl acetate (from 0% to 2%) in petroleum ether to give the title compound (770.0 mg, 3.30 mmol, 87% yield) as a yellow oil.

¹H NMR (400 MHz, CDCl₃) δ=10.53 (s, 1H), 8.26 (d, J=5.2 Hz, 1H), 7.03 (d, J=5.2 Hz, 1H), 4.06 (s, 3H), 0.29 (s, 9H).

### 1-methoxy-2,7-naphthyridine

To a solution of 2-methoxy-4-((trimethylsilyl)ethynyl)nicotinaldehyde (770.0 mg, 3.30 mmol) in MeOH (5 mL) was added NH₃/MeOH (14 M, 5 mL). The mixture was stirred at 50° C. for 3 hrs. The mixture was concentrated in vacuo to give a residue, which was purified by flash column chromatography on silica gel eluting with ethyl acetate (from 0% to 10%) in petroleum ether to give the title compound (280.0 mg, 1.75 mmol, 53% yield) as a yellow solid.

LC-MS(ESI+): m/z 161.1 (M+H)⁺.

### 8-methoxy-1,2,3,4-tetrahydro-2,7-naphthyridine

To a solution of 1-methoxy-2,7-naphthyridine (280.0 mg, 1.75 mmol) in MeOH/HOAc (3 mL/1 mL) was added Pd/C 10% (150.0 mg). The mixture was stirred at 35° C. for 18 hrs under H₂. The mixture was filtered through a Celite® pad and the filtrate was concentrated in vacuo to give the title compound (200.0 mg, 1.22 mmol, 70% yield) as a brown oil, which was used for next step directly.

LC-MS(ESI+): m/z 165.0 (M+H)⁺.

### 8-methoxy-2-(5-(1-methyl-4-(trifluoromethyl)-1H-imidazol-2-yl)pyridin-2-yl)-1,2,3,4-tetrahydro-2,7-naphthyridine

To a solution of 8-methoxy-1,2,3,4-tetrahydro-2,7-naphthyridine (200.0 mg, 1.22 mmol) and 2-fluoro-5-[3-methyl-5-(trifluoromethyl)imidazol-2-yl]pyridine (388.0 mg, 1.58 mmol) in DMSO (3 mL) was added DIEA (315.0 mg, 2.44 mmol) at room temperature. The mixture was stirred at 130° C. for 16 hrs. To the cooled reaction mixture was added water (10 mL) and the aqueous phase was extracted with EtOAc (15 mL×3). The combined organic layers were washed with brine (20 mL×2), dried over Na₂SO₄, filtered through a Celite® pad and the filtrate was concentrated under reduced pressure to give a residue, which was purified by Pre-TLC (PE/EA=1/1) to give the title compound (230.0 mg, 0.59 mmol, 48% yield) as a yellow oil.

LC-MS(ESI+): m/z 390.1 (M+H)⁺.

### 7-(5-(1-methyl-4-(trifluoromethyl)-1H-imidazol-2-yl)pyridin-2-yl)-5,6,7,8-tetrahydro-2,7-naphthyridin-1-ol

To a solution of 8-methoxy-2-(5-(1-methyl-4-(trifluoromethyl)-1H-imidazol-2-yl)pyridin-2-yl)-1,2,3,4-tetrahydro-2,7-naphthyridine (200.0 mg, 0.51 mmol) in DMF (6 mL) were added toluene-4-sulfonic acid (442.0 mg, 2.57 mmol) and LiCl (109.0 mg, 2.57 mmol). The mixture was stirred at 120° C. for 2 hrs. To the cooled reaction mixture was added water (15 mL) and the aqueous phase was extracted with EtOAc (20 mL×2). The combined organic layers were washed with brine (20 mL), dried over Na₂SO₄, filtered through a Celite® pad and the filtrate was concentrated under reduced pressure to give a residue, which was purified by flash column chromatography on silica gel eluting with methanol (from 0% to 5%) in dichloromethane to give the title compound (180.0 mg, 0.48 mmol, 94% yield) as a yellow oil.

LC-MS(ESI+): m/z 376.0 (M+H)⁺.

### 8-chloro-2-(5-(1-methyl-4-(trifluoromethyl)-1H-imidazol-2-yl)pyridin-2-yl)-1,2,3,4-tetrahydro-2,7-naphthyridine

To a solution of 7-(5-(1-methyl-4-(trifluoromethyl)-1H-imidazol-2-yl)pyridin-2-yl)-5,6,7,8-tetrahydro-2,7-naphthyridin-1-ol (180.0 mg, 0.48 mmol) in acetonitrile (4 mL) was added POCl₃ (367.0 mg, 2.40 mmol). The mixture was stirred at 90° C. for 5 hrs. The mixture was concentrated in vacuo to give a residue. The residue was diluted with DCM and NaHCO₃ aqueous solution. The aqueous phase was extracted with DCM (10 mL×2). The combined organic layers were washed with brine (20 mL), dried over Na₂SO₄, filtered through a Celite® pad and the filtrate was concentrated under reduced pressure to give a residue, which was purified by Pre-TLC (PE/EA=0/1) to give the title compound (110.0 mg, 0.28 mmol, 58% yield) as a yellow solid.

LC-MS(ESI+): m/z 394.1 (M+H)⁺.

### 8-(4-cyclopropyl-6-methoxypyrimidin-5-yl)-2-(5-(1-methyl-4-(trifluoromethyl)-1H-imidazol-2-yl)pyridin-2-yl)-1,2,3,4-tetrahydro-2,7-naphthyridine

To a solution of 8-chloro-2-(5-(1-methyl-4-(trifluoromethyl)-1H-imidazol-2-yl)pyridin-2-yl)-1,2,3,4-tetrahydro-2,7-naphthyridine (50.0 mg, 0.13 mmol), 4-cyclopropyl-6-methoxy-5-(4,4,5,5-tetramethyl-1,3,2-dioxaborolan-2-yl)pyrimidine (70.0 mg, 0.25 mmol) in dioxane/H₂O (1 mL/0.2 mL) were added Pd(dppf)Cl₂ (19.0 mg, 0.03 mmol) and K₂CO₃ (35.0 mg, 0.25 mmol) under N₂. The mixture was stirred at 90° C. for 16 hrs under Ar. To the cooled reaction mixture was added water (15 mL) and the aqueous phase was extracted with EtOAc (15 mL×2). The combined organic layers were washed with brine (15 mL), dried over Na₂SO₄, filtered through a Celite® pad and the filtrate was concentrated under reduced pressure to give a residue, which was purified by pre-TLC (PE/EA=0/1) to give the title compound.

LC-MS(ESI+): m/z 508.2 (M+H)⁺.

¹H NMR (400 MHz, DMSO-d₆) δ=8.74 (s, 1H), 8.48 (d, J=5.2 Hz, 1H), 8.40 (d, J=2.0 Hz, 1H), 7.90-7.80 (m, 2H), 7.31 (d, J=4.8 Hz, 1H), 6.92 (d, J=8.8 Hz, 1H), 4.55-4.44 (m, 2H), 3.95-3.87 (m, 2H), 3.85 (s, 3H), 3.74 (s, 3H), 3.10-2.97 (m, 2H), 1.51-1.42 (m, 1H), 1.11-1.01 (m, 2H), 0.91-0.84 (m, 2H).

### Example B12: Synthesis of Compound 3

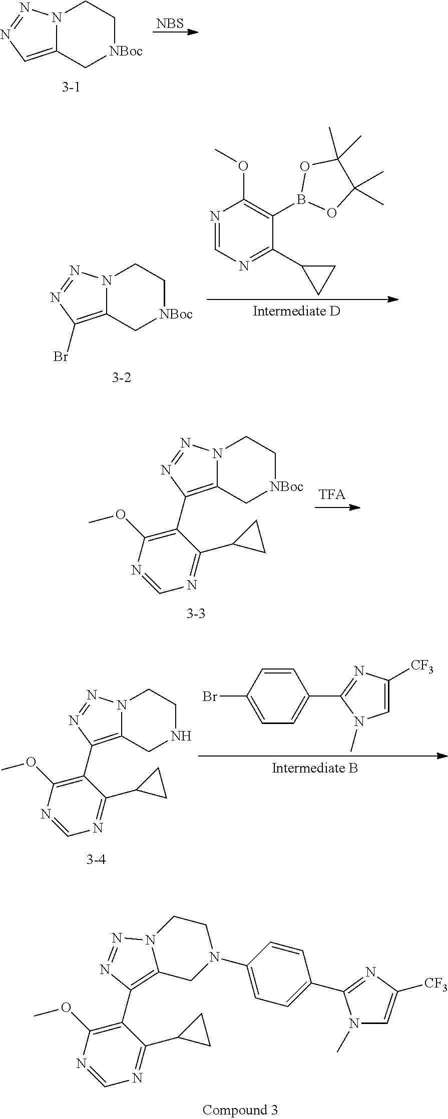

### Tert-butyl 3-bromo-6,7-dihydro-[1,2,3]triazolo[1,5-a]pyrazine-5(4H)-carboxylate

To a solution of 2-methylpropan-2-yl 4,5,6,7-tetrahydro[1,2,3]triazolo[1,5-a]pyrazine-5-carboxylate (500.0 mg, 2.23 mmol) in CH₃CN (10 mL) was added NBS (476.2 mg, 2.68 mmol) at 0° C. The mixture was stirred for 16 hrs at room temperature. The mixture was concentrated to give a residue, which was purified by flash column chromatography eluting with ethyl acetate (from 0% to 10%) in petroleum ether to give the title compound (230.0 mg, 0.76 mmol, 34% yield) as a yellow solid.

LC-MS(ESI+): m/z 303.1 (M+H)⁺.

### Tert-butyl 3-(4-cyclopropyl-6-methoxypyrimidin-5-yl)-6,7-dihydro-[1,2,3]triazolo[1,5-a]pyrazine-5(4H)-carboxylate

A mixture of 2-methylpropan-2-yl 3-bromo-4,5,6,7-tetrahydro[1,2,3]triazolo[1,5-a]pyrazine-5-carboxylate (190.0 mg, 0.63 mmol), 4-cyclopropyl-6-methoxy-5-(4,4,5,5-tetramethyl-1,3,2-dioxaborolan-2-yl)pyrimidine (259.6 mg, 0.94 mmol), CataCXium® A Pd G3 (CAS: 1651823-59-4, 45.6 mg, 0.06 mmol) and K₃PO₄ (399.1 mg, 1.88 mmol) in THF/H₂O (5 mL/0.5 mL) was stirred at 80° C. for 16 hrs under N₂. To the cooled reaction mixture was added water (20 mL) and the aqueous phase was extracted with EtOAc (20 mL×3). The combined organic layers were washed with brine (20 mL), dried over Na₂SO₄, filtered through a Celite® pad and the filtrate was concentrated under reduced pressure to give a residue, which was purified by flash column chromatography on silica gel eluting with ethyl acetate (from 0% to 50%) in petroleum ether to give the title compound (140.0 mg, 0.38 mmol, 60% yield) as a yellow solid.

LC-MS(ESI+): m/z 373.1 (M+H)⁺.

### 3-(4-cyclopropyl-6-methoxypyrimidin-5-yl)-4,5,6,7-tetrahydro-[1,2,3]triazolo[1,5-a]pyrazine

To a solution of 2-methylpropan-2-yl 3-(6-cyclopropyl-4-methoxypyrimidin-5-yl)-4,5,6,7-tetrahydro[1,2,3]triazolo[1,5-a]pyrazine-5-carboxylate (130.0 mg, 0.35 mmol) in DCM (3 mL) was added TFA (0.5 mL). The mixture was stirred at room temperature for 2 hrs. The volatiles were removed in vacuo. The residue was dissolved in DCM (15 mL), then pH was adjusted to 7-8 by adding NaHCO₃ powder. The mixture was filtrated through a Celite® pad and the filtrate was concentrated to give a residue, which was purified by flash column chromatography on silica gel eluting with MeOH (from 0% to 5%) in DCM to give the title compound (90.0 mg, 0.33 mmol, 94% yield) as a yellow oil.

LC-MS(ESI+): m/z 273.1 (M+H)⁺.

### 3-(4-cyclopropyl-6-methoxypyrimidin-5-yl)-5-(4-(1-methyl-4-(trifluoromethyl)-1H-imidazol-2-yl)phenyl)-4,5,6,7-tetrahydro-[1,2,3]triazolo[1,5-a]pyrazine

To a mixture of 3-(6-cyclopropyl-4-methoxypyrimidin-5-yl)-4,5,6,7-tetrahydro[1,2,3]triazolo[1,5-a]pyrazine (85.0 mg, 0.31 mmol), 2-(4-bromophenyl)-3-methyl-5-(trifluoromethyl)imidazole (190.5 mg, 0.62 mmol), t-BuONa (240.0 mg, 2.50 mmol) and 2-dicyclohexylphosphino-2′,4′,6′-triisopropylbiphenyl (X-PHOS) (297.6 mg, 0.62 mmol) in toluene (5 mL) was added Pd(OAc)₂ (7.0 mg, 0.03 mmol) under N₂. The mixture was stirred at 100° C. for 16 hrs. To the reaction mixture was added water (10 mL) and the aqueous phase was extracted with EtOAc (20 mL×2). The combined organic layers were washed with brine (20 mL), dried over Na₂SO₄, filtered through a Celite® pad and the filtrate was concentrated under reduced pressure to give a residue, which was purified by pre-TLC (PE/EA=1/3) to give the title compound.

LC-MS(ESI+): m/z 497.2 (M+H)⁺.

¹H NMR (400 MHz, DMSO-d₆) δ=8.68 (s, 1H), 7.86 (s, 1H), 7.59 (d, J=8.8 Hz, 2H), 7.20 (d, J=9.2 Hz, 2H), 4.64-4.47 (m, 4H), 4.10-3.97 (m, 2H), 3.94 (s, 3H), 3.75 (s, 3H), 2.20-2.07 (m, 1H), 1.13-1.04 (m, 2H), 0.98-0.88 (m, 2H).

### Example B13: Synthesis of Compound 4

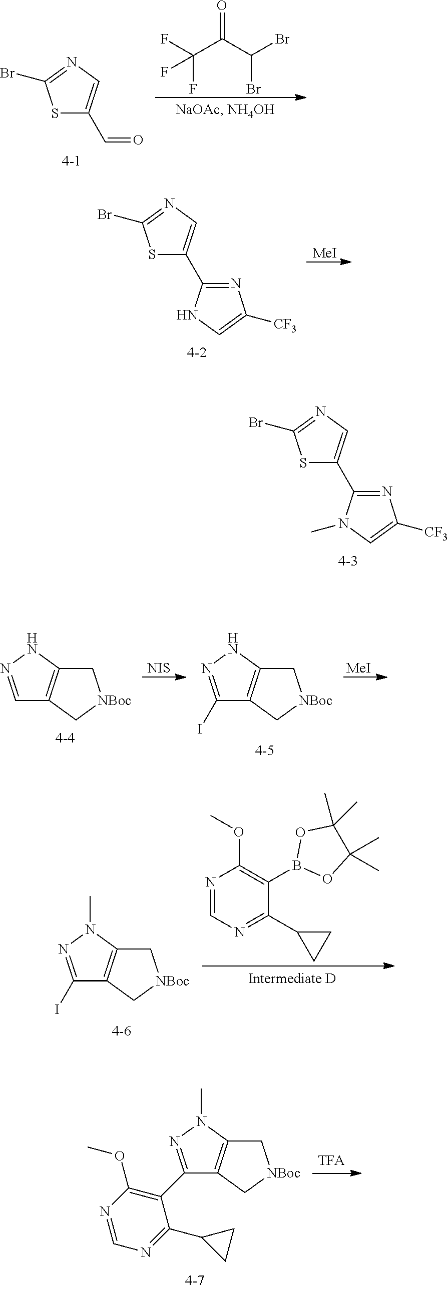

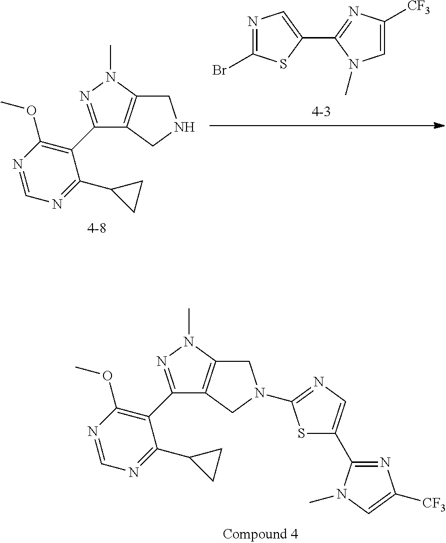 [SMILES: COc1ncnc(C2CC2)c1-c1nn(C)c2c1CNC2 ; Cn1cc(C(F)(F)F)nc1-c1cnc(Br)s1 ; COc1ncnc(C2CC2)c1-c1nn(C)c2c1CN(c1ncc(-c3nc(C(F)(F)F)cn3C)s1)C2]

### 2-bromo-5-(4-(trifluoromethyl)-1H-imidazol-2-yl)thiazole

To a solution of sodium acetate (0.85 g, 10.42 mmol) in water (15 mL) was added 3,3-dibromo-1,1,1-trifluoropropan-2-one (2.11 g, 7.81 mmol) dropwise at 0° C. Then, the mixture was stirred for 1 hr at 100° C. After cooling to room temperature, a mixture of 2-bromo-1,3-thiazole-5-carbaldehyde (1.00 g, 5.21 mmol), MeOH (30 mL) and ammonium hydroxide (28%, 10 mL) was added to the solution and the resulting mixture was stirred for 16 hrs at room temperature. The mixture was concentrated to remove MeOH. The residue was diluted with water (20 mL), extracted with EtOAc (25 mL×3). The combined organic fractions were washed with brine (20 mL), dried over Na₂SO₄, filtered through a Celite® pad and concentrated under the reduced pressure to give a residue, which was purified by flash column chromatography on silica gel (ethyl acetate in petroleum ether from 0% to 25%) to give the tittle compound (470.0 mg, 1.58 mmol, 30% yield) as a yellow solid.

¹H NMR (400 MHz, CDCl₃) δ=9.87 (br.s, 1H), 7.79 (s, 1H), 7.37 (s, 1H).

### 2-bromo-5-(1-methyl-4-(trifluoromethyl)-1H-imidazol-2-yl)thiazole

To a solution of 2-(2-bromo-1,3-thiazol-5-yl)-5-(trifluoromethyl)-3H-imidazole (470 mg, 1.58 mmol) and Cs₂CO₃ (1284.2 mg, 3.94 mmol) in DMF (10 mL) was added Mel (447.5 mg, 3.15 mmol) dropwise at 0° C. The mixture was gradually warmed to room temperature and stirred for 16 hrs. To the reaction mixture was added water (10 mL) and the aqueous phase was extracted with EtOAc (10 mL×2). The combined organic layers were washed with brine (20 mL), dried over Na₂SO₄, filtered through a Celite® pad and the filtrate was concentrated under reduced pressure to give a residue, which was purified by flash column chromatography on silica gel eluting with ethyl acetate (from 0% to 20%) in petroleum ether to give the title compound (350.0 mg, 1.12 mmol, 71% yield) as a yellow solid.

LC-MS(ESI+): m/z 313.9 (M+H)⁺.

### Tert-butyl 3-iodo-4,6-dihydropyrrolo[3,4-c]pyrazole-5(1H)-carboxylate

To a solution of 2-methylpropan-2-yl 1,4,5,6-tetrahydropyrrolo[4,3-c]pyrazole-5-carboxylate (2.50 g, 11.95 mmol) in DMF (30 mL) was added NIS (3.49 g, 15.53 mmol). The mixture was stirred at 60° C. for 6 hrs. After cooling to room temperature, sat. Na₂SO₃ solution (50 mL) was added into the mixture slowly, then the aqueous phase was extracted with EtOAc (50 mL×3). The combined organic fractions were washed with brine (60 mL×3), dried over Na₂SO₄, filtered through a Celite® pad and the filtrate was concentrated under reduced pressure to give a residue, which was recrystallized from Et₂O (30 mL) to give the tittle compound (2.85 g, 8.50 mmol, 71% yield) as a yellow solid.

¹H NMR (400 MHz, CDCl₃) δ=4.65-4.50 (m, 2H), 4.44-4.30 (m, 2H), 1.51 (s, 9H).

### Tert-butyl 3-iodo-1-methyl-4,6-dihydropyrrolo[3,4-c]pyrazole-5(1H)-carboxylate

To a solution of 2-methylpropan-2-yl 3-iodo-1,4,5,6-tetrahydropyrrolo[4,3-c]pyrazole-5-carboxylate (2.85 g, 8.50 mmol) and Cs₂CO₃ (5.54 g, 17.01 mmol) in DMF (30 mL) was added iodomethane (2.41 g, 17.01 mmol) dropwise at 0° C. The mixture was gradually warmed to room temperature and stirred for 16 hrs. To the reaction mixture was added water (50 mL) and the aqueous phase was extracted with EtOAc (50 mL×3). The combined organic layers were washed with brine (50 mL×2), dried over Na₂SO₄, filtered through a Celite® pad and the filtrate was concentrated under reduced pressure to give a residue, which was purified by flash column chromatography on silica gel eluting with ethyl acetate (from 0% to 30%) in petroleum ether to give the title compound (760.0 mg, 2.18 mmol, 26% yield) as a yellow solid.

¹H NMR (400 MHz, CDCl₃) δ=4.45-4.41 (m, 2H), 4.26-4.22 (m, 2H), 3.75 (s, 3H), 1.43 (s, 9H).

### Tert-butyl 3-(4-cyclopropyl-6-methoxypyrimidin-5-yl)-1-methyl-4,6-dihydropyrrolo[3,4-c]pyrazole-5(1H)-carboxylate

A mixture of 2-methylpropan-2-yl 3-iodo-1-methyl-5,6-dihydro-4H-pyrrolo[4,3-c]pyrazole-5-carboxylate (600.0 mg, 1.72 mmol), 4-cyclopropyl-6-methoxy-5-(4,4,5,5-tetramethyl-1,3,2-dioxaborolan-2-yl)pyrimidine (949.0 mg, 3.44 mmol), cataCXium® A Pd G3 (12.5 mg, 0.017 mmol) and K₃PO₄ (730.0 mg, 3.44 mmol) in THF/water (10 mL/1.5 mL) was stirred at 80° C. for 16 hrs under N₂. To the cooled reaction mixture was added water (20 mL) and the aqueous phase was extracted with EtOAc (20 mL×2). The combined organic layers were washed with brine (30 mL), dried over Na₂SO₄, filtered through a Celite® pad and the filtrate was concentrated under reduced pressure to give a residue, which was purified by flash column chromatography on silica gel eluting with ethyl acetate (from 0% to 60%) in petroleum ether to give the title compound (600.0 mg, 1.62 mmol, 94% yield) as a yellow oil.

LC-MS(ESI+): m/z 372.2 (M+H)⁺.

### 3-(4-cyclopropyl-6-methoxypyrimidin-5-yl)-1-methyl-1,4,5,6-tetrahydropyrrolo[3,4-c]pyrazole

To a solution of 2-methylpropan-2-yl 3-(6-cyclopropyl-4-methoxypyrimidin-5-yl)-1-methyl-5,6-dihydro-4H-pyrrolo[4,3-c]pyrazole-5-carboxylate (200.0 mg, 0.54 mmol) in DCM (20 mL) was added TFA (3 mL) at 0° C. The mixture was stirred at room temperature for 5 hrs. The mixture was concentrated to give the tittle compound (140.0 mg, 0.52 mmol, 96% yield) as a yellow oil, which was used for next step without further work up.

LC-MS(ESI+): m/z 272.1 (M+H)⁺.

### 2-(3-(4-cyclopropyl-6-methoxypyrimidin-5-yl)-1-methyl-4,6-dihydropyrrolo[3,4-c]pyrazol-5(1H)-yl)-5-(1-methyl-4-(trifluoromethyl)-1H-imidazol-2-yl)thiazole

A mixture of 3-(6-cyclopropyl-4-methoxypyrimidin-5-yl)-1-methyl-5,6-dihydro-4H-pyrrolo[4,3-c]pyrazole (146.0 mg, 0.54 mmol), 2-(2-bromo-1,3-thiazol-5-yl)-3-methyl-5-(trifluoromethyl)imidazole (218.3 mg, 0.70 mmol) and DIEA (139.6 mg, 1.08 mmol) in DMSO (3 mL) was stirred at 90° C. for 16 hrs. To the cooled reaction mixture was added water (20 mL) and the aqueous phase was extracted with EtOAc (20 mL×2). The combined organic layers were washed with brine (30 mL×2), dried over Na₂SO₄, filtered through a Celite® pad and the filtrate was concentrated under reduced pressure to give a residue, which was purified by flash chromatography on C₁₈ column eluting with ACN (from 0% to 100%) in water to give the title compound.

LC-MS(ESI+): m/z 503.2 (M+H)⁺.

¹H NMR (400 MHz, DMSO-d₆) δ=8.59 (s, 1H), 7.85 (s, 1H), 7.69 (s, 1H), 4.79 (s, 2H), 4.49 (s, 2H), 3.94 (s, 3H), 3.89 (s, 3H), 3.84 (s, 3H), 2.81-2.60 (m, 1H), 1.15-0.88 (m, 4H).

### Example B14: Synthesis of Compound 5

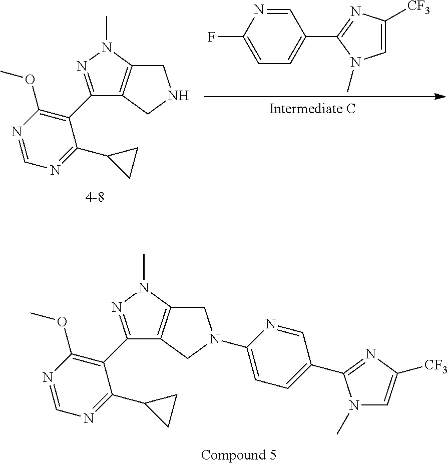 [SMILES: COc1ncnc(C2CC2)c1-c1nn(C)c2c1CNC2 ; Cn1cc(C(F)(F)F)nc1-c1ccc(F)nc1 ; COc1ncnc(C2CC2)c1-c1nn(C)c2c1CN(c1ccc(-c3nc(C(F)(F)F)cn3C)cn1)C2]

### 3-(4-cyclopropyl-6-methoxypyrimidin-5-yl)-1-methyl-5-(5-(1-methyl-4-(trifluoromethyl)-1H-imidazol-2-yl)pyridin-2-yl)-1,4,5,6-tetrahydropyrrolo[3,4-c]pyrazole

The solution of 3-(6-cyclopropyl-4-methoxypyrimidin-5-yl)-1-methyl-5,6-dihydro-4H-pyrrolo[4,3-c]pyrazole (80.0 mg, 0.30 mmol) and DIEA (76.3 mg, 0.59 mmol) in DMSO (2 mL) was added 2-fluoro-5-[3-methyl-5-(trifluoromethyl)imidazol-2-yl]pyridine (144.7 mg, 0.59 mmol). The mixture was stirred at 130° C. for 6 hrs. After cooling to room temperature, to the reaction mixture was added water (10 mL) and the aqueous phase was extracted with EtOAc (10 mL×3). The combined organic layers were washed with brine (20 mL), dried over Na₂SO₄, filtered through a Celite® pad and the filtrate was concentrated under reduced pressure to give a residue, which was purified by flash chromatography on C₁₈ column eluting with ACN (from 0% to 100%) in water to give the title compound.

LC-MS(ESI−): m/z 497.2 (M+H)⁺.

¹H NMR (400 MHz, DMSO-d₆) δ=8.59 (s, 1H), 8.47 (d, J=2.4 Hz, 1H), 7.92-7.89 (m, 2H), 6.67 (d, J=8.8 Hz, 1H), 4.78 (s, 2H), 4.49 (s, 2H), 3.94 (s, 3H), 3.90 (s, 3H), 3.77 (s, 3H), 2.72-2.60 (m, 1H), 1.10-0.96 (m, 4H).

### Example B15: Synthesis of Compound 6

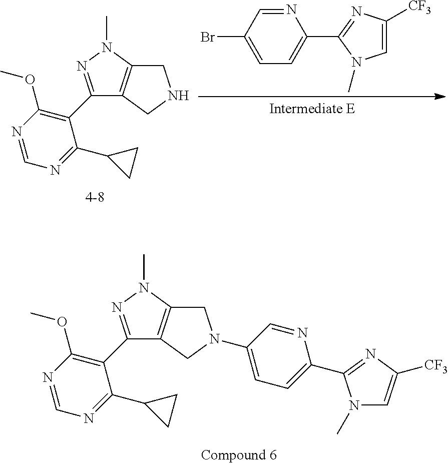 [SMILES: COc1ncnc(C2CC2)c1-c1nn(C)c2c1CNC2 ; Cn1cc(C(F)(F)F)nc1-c1ccc(Br)cn1 ; COc1ncnc(C2CC2)c1-c1nn(C)c2c1CN(c1ccc(-c3nc(C(F)(F)F)cn3C)nc1)C2]

### 3-(4-cyclopropyl-6-methoxypyrimidin-5-yl)-1-methyl-5-(6-(1-methyl-4-(trifluoromethyl)-1H-imidazol-2-yl)pyridin-3-yl)-1,4,5,6-tetrahydropyrrolo[3,4-c]pyrazole

To a solution of 3-(4-cyclopropyl-6-methoxypyrimidin-5-yl)-1-methyl-1,4,5,6-tetrahydropyrrolo[3,4-c]pyrazole (85.0 mg, 0.31 mmol) and 5-bromo-2-[3-methyl-5-(trifluoromethyl)imidazol-2-yl]pyridine (144.0 mg, 0.47 mmol) in toluene (3 mL) were added Pd(OAc)₂ (7.0 mg, 0.03 mmol), X-PHOS (299.0 mg, 0.63 mmol) and sodium tert-butoxide (60.0 mg, 0.63 mmol). The mixture was stirred at 100° C. for 16 hrs under Ar. To the cooled reaction mixture was added water (15 mL) and the aqueous phase was extracted with EtOAc (15 mL×2). The combined organic layers were washed with brine (10 mL), dried over Na₂SO₄, filtered through a Celite® pad and the filtrate was concentrated under reduced pressure to give a residue, which was purified by Pre-TLC (DCM/EA=1/1) to give the title compound.

LC-MS(ESI+): m/z 497.1 (M+H)⁺.

¹H NMR (400 MHz, DMSO-d₆) δ=8.60 (s, 1H), 8.07 (d, J=2.8 Hz, 1H), 7.94 (d, J=8.8 Hz, 1H), 7.87 (s, 1H), 7.09-7.21 (m, 1H), 4.69 (s, 2H), 4.44 (s, 2H), 4.05 (s, 3H), 3.95 (s, 3H), 3.90 (s, 3H), 2.70-2.58 (m, 1H), 1.08-1.04 (m, 2H), 0.99-0.95 (m, 2H).

### Example B16: Synthesis of Compound 7

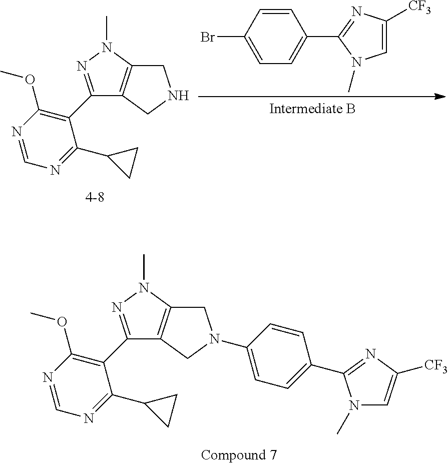 [SMILES: COc1ncnc(C2CC2)c1-c1nn(C)c2c1CNC2 ; Cn1cc(C(F)(F)F)nc1-c1ccc(Br)cc1 ; COc1ncnc(C2CC2)c1-c1nn(C)c2c1CN(c1ccc(-c3nc(C(F)(F)F)cn3C)cc1)C2]

### 3-(4-cyclopropyl-6-methoxypyrimidin-5-yl)-1-methyl-5-(4-(1-methyl-4-(trifluoromethyl)-1H-imidazol-2-yl)phenyl)-1,4,5,6-tetrahydropyrrolo[3,4-c]pyrazole

To a solution of 3-(4-cyclopropyl-6-methoxypyrimidin-5-yl)-1-methyl-1,4,5,6-tetrahydropyrrolo[3,4-c]pyrazole (85.0 mg, 0.31 mmol) and 2-(4-bromophenyl)-3-methyl-5-(trifluoromethyl)imidazole (143.0 mg, 0.47 mmol) in toluene (3 mL) were added Pd(OAc)₂ (7.0 mg, 0.03 mmol), X-PHOS (299.0 mg, 0.63 mmol) and sodium tert-butoxide (60.0 mg, 0.63 mmol). The mixture was stirred at 100° C. for 16 hrs under Ar. To the cooled reaction mixture was added water (15 mL) and extracted with EtOAc (15 mL×2). The combined organic layers were washed with brine (15 mL), dried over Na₂SO₄, filtered through a Celite® pad and the filtrate was concentrated under reduced pressure to give a residue, which was purified by Pre-TLC (DCM/EA=1/1) to give the title compound.

LC-MS(ESI+): m/z 496.1 (M+H)⁺.

¹H NMR (400 MHz, DMSO-d₆) δ=8.60 (s, 1H), 7.83 (d, J=1.2 Hz, 1H), 7.59 (d, J=8.8 Hz, 2H), 6.73 (d, J=8.8 Hz, 2H), 4.64 (s, 2H), 4.38 (s, 2H), 3.94 (s, 3H), 3.90 (s, 3H), 3.76 (s, 3H), 2.70-2.55 (m, 1H), 1.16-1.02 (m, 2H), 0.99-0.90 (m, 2H).

### Example B17: Synthesis of Compound 8

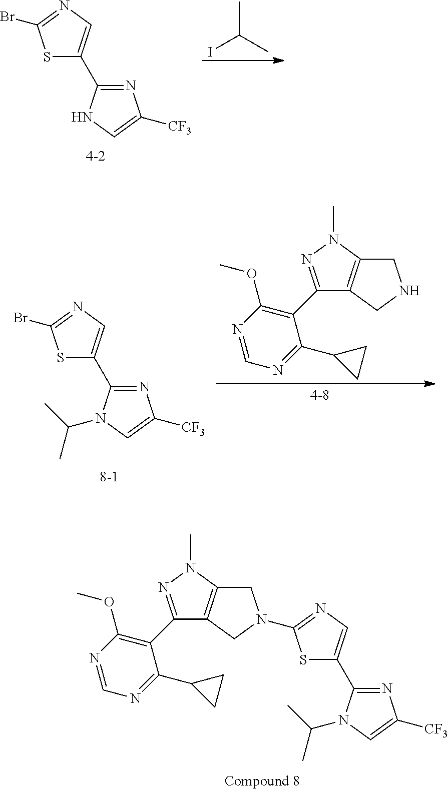

### 2-bromo-5-(1-isopropyl-4-(trifluoromethyl)-1H-imidazol-2-yl)thiazole

To a solution of 2-(2-bromo-1,3-thiazol-5-yl)-5-(trifluoromethyl)-3H-imidazole (300.0 mg, 1.01 mmol) in DMF (4 mL) was added Cs₂CO₃ (819.7 mg, 2.52 mmol) and 2-iodopropane (341.7 mg, 2.01 mmol) at room temperature. The mixture was stirred at 60° C. for 16 hrs. To the cooled reaction mixture was added water (10 mL) and the aqueous phase was extracted with EtOAc (10 mL×2). The combined organic layers were washed with brine (20 mL×2), dried over Na₂SO₄, filtered through a Celite® pad and the filtrate was concentrated under reduced pressure to give a residue, which was purified by flash column chromatography on silica gel eluting with ethyl acetate (from 0% to 12%) in petroleum ether to give the title compound (150.0 mg, 0.44 mmol, 44% yield) as a yellow oil.

LC-MS(ESI+): m/z 340.0 (M+H)⁺.

### 2-(3-(4-cyclopropyl-6-methoxypyrimidin-5-yl)-1-methyl-4,6-dihydropyrrolo[3,4-c]pyrazol-5(1H)-yl)-5-(1-isopropyl-4-(trifluoromethyl)-1H-imidazol-2-yl)thiazole

To a solution of 3-(4-cyclopropyl-6-methoxypyrimidin-5-yl)-1-methyl-1,4,5,6-tetrahydropyrrolo[3,4-c]pyrazole (50.0 mg, 0.18 mmol) in DMSO (2 mL) were added 2-(2-bromo-1,3-thiazol-5-yl)-3-(prop-2-yl)-5-(trifluoromethyl)imidazole (81.0 mg, 0.24 mmol) and DIEA (48.0 mg, 0.37 mmol) at room temperature. The mixture was stirred at 90° C. for 16 hours. To the cooled reaction mixture was added water (20 mL) and the aqueous phase was extracted with EtOAc (20 mL×3). The combined organic layers were washed with brine (20 mL×2), dried over Na₂SO₄, filtered through a Celite® pad and the filtrate was concentrated under reduced pressure to give a residue, which was purified by pre-TLC (PE/EA=1/1) to give the title compound.

LC-MS(ESI+): m/z 531.1 (M+H)⁺.

¹H NMR (400 MHz, DMSO-d₆) δ=8.59 (s, 1H), 8.08 (s, 1H), 7.61 (s 1H), 4.79 (s, 2H), 4.75-4.68 (m, 1H), 4.49 (s, 2H), 3.94 (s, 3H), 3.89 (s, 3H), 2.71-2.67 (m, 1H), 1.45 (d, J=6.4 Hz, 6H), 1.07-1.04 (m, 2H), 0.99-0.95 (m, 2H).

### Example B18: Synthesis of Compound 9

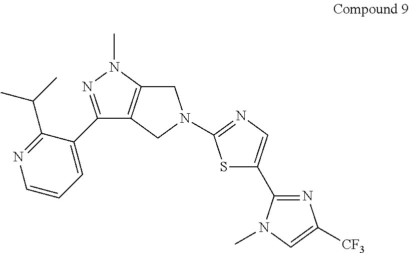 [SMILES: CC(C)c1ncccc1-c1nn(C)c2c1CN(c1ncc(-c3nc(C(F)(F)F)cn3C)s1)C2]

The synthetic route for Compound 9 was similar to that of Compound 4 by replacing intermediate D with 2-isopropyl-3-(4,4,5,5-tetramethyl-1,3,2-dioxaborolan-2-yl)pyridine (intermediate F). The crude product was purified by prep-TLC (PE/EA=1/1) to give the title compound.

LC-MS(ESI+): m/z 474.1 (M+H)⁺.

¹H NMR (400 MHz, DMSO-d₆) δ 8.55-8.53 (m, 1H), 7.86 (s, 1H), 7.76-7.70 (m, 1H), 7.70 (s, 1H), 7.32-7.25 (m, 1H), 4.79 (s, 2H), 4.65 (s, 2H), 3.90 (s, 3H), 3.84 (s, 3H), 3.78-3.71 (m, 1H), 1.23-1.18 (m, 6H).

### Example B19: Synthesis of Compound 10

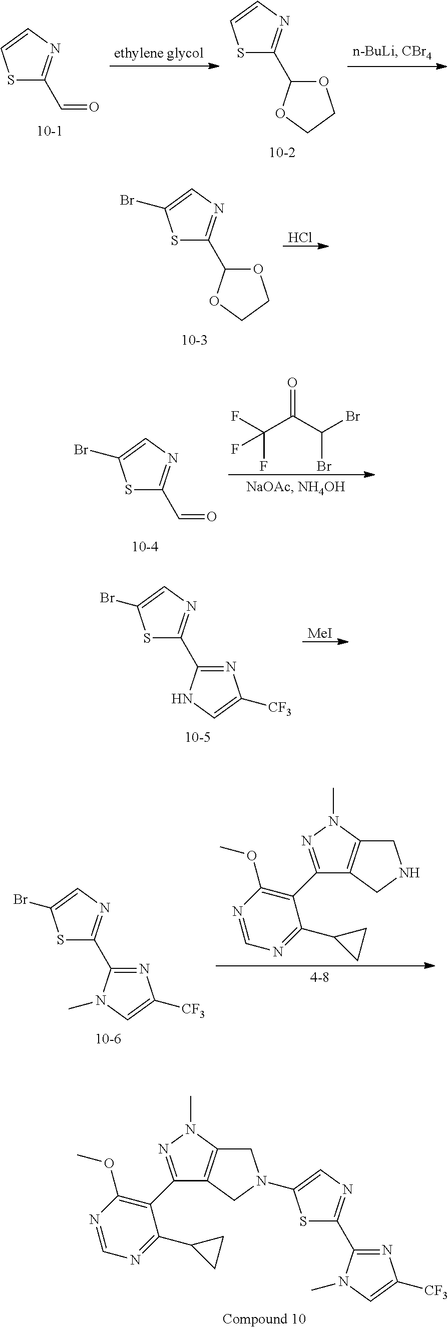

### 2-(1,3-dioxolan-2-yl)thiazole

To a solution of ethylene glycol (5.50 g, 88.42 mmol), 1,3-thiazole-2-carbaldehyde (5.00 g, 44.21 mmol) in toluene (50 mL) was added toluene-4-sulfonic acid (800.0 mg, 4.65 mmol), the reaction was stirred at 120° C. for 16 hrs. To the cooled reaction mixture was added water (100 mL) and extracted with EtOAc (60 mL×3). The combined organic layers were washed with brine (80 mL), dried over Na₂SO₄, filtered through a Celite® pad and the filtrate was concentrated under reduced pressure to give a residue, which was purified by flash column chromatography on silica gel eluting with ethyl acetate (from 0% to 150%) in petroleum ether to give the title compound (5.20 g, 33.08 mmol, 750% yield) as a yellow oil.

¹H NMR (400 MHz, CDCl₃) δ=7.83 (d, J=3.2 Hz, 1H), 7.39 (d, J=3.2 Hz, 1H), 6.17 (s, 1H), 4.23-4.13 (m, 2H), 4.10-4.00 (m, 2H).

### 5-bromo-2-(1,3-dioxolan-2-yl)thiazole

To a solution of 2-(1,3-dioxolan-2-yl)thiazole (4.20 g, 26.72 mmol) in dry THF (40 mL) was added n-BuLi (2.5 M, 13 mL, 32.5 mmol) dropwise at −78° C. Then, the mixture was stirred at −78° C. for 30 min and CBr₄ (26.60 g, 80.15 mmol) was added dropwise. The mixture was warmed to 0° C. and stirred for 2 hr. To the reaction mixture was added water (50 mL) slowly at 0° C. The aqueous phase was extracted with EtOAc (50 mL×3). The combined organic layers were washed with brine (30 mL), dried over Na₂SO₄, filtered through a Celite® pad and the filtrate was concentrated under reduced pressure to give a residue, which was purified by flash column chromatography on silica gel eluting with ethyl acetate (from 0% to 5%) in petroleum ether to give the title compound (5.30 g, 22.45 mmol, 84% yield) as a brown oil.

¹H NMR (400 MHz, CDCl₃) δ=7.69 (s, 1H), 6.06 (s, 1H), 4.16-4.11 (m, 2H), 4.10-4.05 (m, 2H).

### 5-bromothiazole-2-carbaldehyde

To a solution of 5-bromo-2-(1,3-dioxolan-2-yl)thiazole (6.50 g, 27.53 mmol) in THF (50 mL) was added HCl (1 M, 30 mL). The mixture was stirred at 70° C. for 16 hrs. After cooling to room temperature, the mixture was neutralized with saturated aqueous NaHCO₃ solution. The mixture was extracted with EtOAc (50 mL×2). The combined organic layers were washed with brine (80 mL), dried over Na₂SO₄, filtered through a Celite® pad and the filtrate was concentrated under reduced pressure to give a residue, which was purified by flash column chromatography on silica gel eluting with ethyl acetate (from 0% to 5%) in petroleum ether to give the title compound (3.80 g, 19.79 mmol, 72% yield) as a yellow oil.

¹H NMR (400 MHz, CDCl₃) δ=9.87 (s, 1H), 8.02 (s, 1H).

### 5-bromo-2-(4-(trifluoromethyl)-1H-imidazol-2-yl)thiazole

To a solution of sodium acetate (2.82 g, 34.36 mmol) in water (60 mL) was added 3,3-dibromo-1,1,1-trifluoropropan-2-one (6.96 g, 25.78 mmol) dropwise at 0° C. After the addition, the mixture was stirred for 1 hr at 100° C. After cooling to room temperature, a solution of 5-bromo-1,3-thiazole-2-carbaldehyde (3.30 g, 17.19 mmol), MeOH (120 mL), and ammonium hydroxide (28%, 40 mL) was added to the solution and the resulting mixture was stirred for 16 hrs at room temperature. The volatiles were removed in vacuo. The residue was diluted with water (50 mL), and the aqueous phase was extracted with EtOAc (50 mL×3). The combined organic layers were washed with brine (100 mL), dried over Na₂SO₄, filtered through a Celite® pad and the filtrated was concentrated under reduced pressure to give a residue, which was purified by flash column chromatography on silica gel eluting with ethyl acetate (from 0% to 25%) in petroleum ether to give the title compound (1.57 g, 5.27 mmol, 31% yield) as a yellow oil.

LC-MS(ESI+): m/z 297.8 (M+H)⁺.

### 5-bromo-2-(1-methyl-4-(trifluoromethyl)-1H-imidazol-2-yl)thiazole

To a solution of 5-bromo-2-[5-(trifluoromethyl)-3H-imidazol-2-yl]-1,3-thiazole (400 mg, 1.34 mmol) and Cs₂CO₃ (1092.9 mg, 3.35 mmol) in DMF (6 mL) was added iodomethane (380.4 mg, 2.68 mmol) dropwise at 0° C. The mixture was gradually warmed to room temperature and stirred for 16 hrs. To the reaction mixture was added water (20 mL) and the aqueous phase was extracted with EtOAc (20 mL×2). The combined organic layers were washed with brine (20 mL×2), dried over Na₂SO₄, filtered through a Celite® pad and the filtrate was concentrated under reduced pressure to give a residue, which was purified by flash column chromatography on silica gel eluting with ethyl acetate (from 0% to 10%) in petroleum ether to give the title compound (280.0 mg, 0.90 mmol, 67% yield) as a yellow solid.

LC-MS(ESI+): m/z 311.9 (M+H)⁺.

### 5-(3-(4-cyclopropyl-6-methoxypyrimidin-5-yl)-1-methyl-4,6-dihydropyrrolo[3,4-c]pyrazol-5(1H)-yl)-2-(1-methyl-4-(trifluoromethyl)-1H-imidazol-2-yl)thiazole

To a solution of 5-bromo-2-(1-methyl-4-(trifluoromethyl)-1H-imidazol-2-yl)thiazole (81.1 mg, 0.26 mmol) and 3-(4-cyclopropyl-6-methoxypyrimidin-5-yl)-1-methyl-1,4,5,6-tetrahydropyrrolo[3,4-c]pyrazole (84.1 mg, 0.31 mmol) in toluene (2 mL) were added Pd₂(dba)₃ (24.0 mg, 0.03 mmol), BINAP (80.0 mg, 0.13 mmol) and sodium tert-butoxide (50.0 mg, 0.52 mmol). The mixture was stirred at 80° C. for 16 hours under Ar. To the cooled reaction mixture was added water (15 mL) and the aqueous phase was extracted with EtOAc (15 mL×2). The combined organic layers were washed with brine (15 mL), dried over Na₂SO₄, filtered through a Celite® pad and the filtrate was concentrated under reduced pressure to give a residue, which was purified by Pre-TLC (DCM/EA=1/1) to give the title compound.

LC-MS(ESI+): m/z 503.1 (M+H)⁺.

¹H NMR (400 MHz, DMSO-d₆) δ=8.59 (s, 1H), 7.94 (s, 1H), 6.94 (s, 1H), 4.65 (s, 2H), 4.38 (s, 2H), 4.01 (s, 3H), 3.94 (s, 3H), 3.88 (s, 3H), 2.73-2.61 (m, 1H), 1.10-1.04 (m, 2H), 0.98-0.90 (m, 2H).

### Example B20: Synthesis of Compound 11

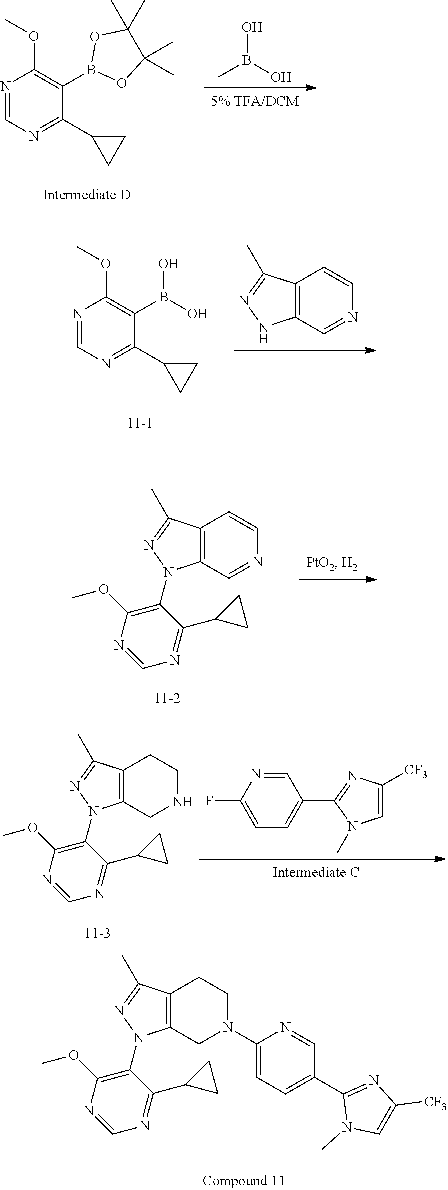

### (4-cyclopropyl-6-methoxypyrimidin-5-yl)boronic Acid

To a solution of 4-cyclopropyl-6-methoxy-5-(4,4,5,5-tetramethyl-1,3,2-dioxaborolan-2-yl)pyrimidine (2.00 g, 7.24 mmol) in 5% (v/v) TFA/DCM (20 mL) was added methylboronic acid (2.16 g, 36.22 mmol). The mixture was stirred at room temperature for 18 hrs. The volatiles were removed under reduced pressure to give a residue, which was purified by reversed phase column (H₂O/ACN from 100/0 to 4/1) to give the title compound (850.0 mg, 4.38 mmol, 60% yield) as a white solid.

LC-MS(ESI+): m/z 195.1 (M+H)⁺.

### 1-(4-cyclopropyl-6-methoxypyrimidin-5-yl)-3-methyl-1H-pyrazolo[3,4-c]pyridine

To a solution of 3-methyl-1H-pyrazolo[3,4-c]pyridine (250.0 mg, 1.88 mmol), (4-cyclopropyl-6-methoxypyrimidin-5-yl)bornanediol (728.2 mg, 3.75 mmol) in dioxane (8 mL) were added Copper(II) acetate monohydrate (112.4 mg, 0.56 mmol) and DBU (571.3 mg, 3.75 mmol). The mixture was stirred at 70° C. for 16 hrs. To the cooled reaction mixture was added water (40 mL) and the aqueous phase was extracted with EtOAc (40 mL×3). The combined organic layers were washed with brine (30 mL×2), dried over Na₂SO₄, filtered through a Celite® pad and the filtrate was concentrated under reduced pressure to give a residue, which was purified by pre-TLC (PE/EA=1/1) to give the title compound (80.0 mg, 0.28 mmol, 15% yield) as a yellow oil.

LC-MS(ESI+): m/z 282.1 (M+H)⁺.

### 1-(4-cyclopropyl-6-methoxypyrimidin-5-yl)-3-methyl-4,5,6,7-tetrahydro-1H-pyrazolo[3,4-c]pyridine

A mixture of 1-(6-cyclopropyl-4-methoxypyrimidin-5-yl)-3-methylpyrazolo[3,4-c]pyridine (70.0 mg, 0.25 mmol) and Platinum(IV) oxide (33.9 mg, 0.15 mmol) in EtOH (3 mL) was stirred at room temperature for 16 hrs under H₂. The mixture was filtered and the filtrate was concentrated under reduced pressure to give the title compound (50.0 mg, 0.18 mmol, 72% yield) as a yellow oil.

LC-MS(ESI+): m/z 286.2 (M+H)⁺.

### 1-(4-cyclopropyl-6-methoxypyrimidin-5-yl)-3-methyl-6-(5-(1-methyl-4-(trifluoromethyl)-1H-imidazol-2-yl)pyridin-2-yl)-4,5,6,7-tetrahydro-1H-pyrazolo[3,4-c]pyridine

A mixture of 1-(6-cyclopropyl-4-methoxypyrimidin-5-yl)-3-methyl-4,5,6,7-tetrahydropyrazolo[3,4-c]pyridine (60.0 mg, 0.21 mmol), 2-fluoro-5-[3-methyl-5-(trifluoromethyl)imidazol-2-yl]pyridine (103.1 mg, 0.42 mmol) and DIEA (108.6 mg, 0.84 mmol) in DMSO (3 mL) was stirred at 130° C. for 16 hrs. To the cooled reaction mixture was added water (20 mL) and the aqueous phase was extracted with EtOAc (20 mL×3). The combined organic layers were washed with brine (30 mL×2), dried over Na₂SO₄, filtered through a Celite® pad and the filtrate was concentrated under reduced pressure to give a residue, which was purified by pre-TLC (PE/EA=1/1) to give the title compound.

LC-MS(ESI+): m/z 511.1 (M+H)⁺.

¹H NMR (400 MHz, DMSO-d₆) δ=8.72 (s, 1H), 8.41 (d, J=2.0 Hz, 1H), 7.95-7.85 (m, 2H), 7.08 (d, J=8.8 Hz, 1H), 4.58-4.45 (m, 2H), 4.03-3.85 (m, 5H), 3.74 (s, 3H), 2.72-2.58 (m, 2H), 2.16 (s, 3H), 1.65-1.54 (m, 1H), 1.14-0.98 (m, 4H).

### Example B21: Synthesis of Compound 12

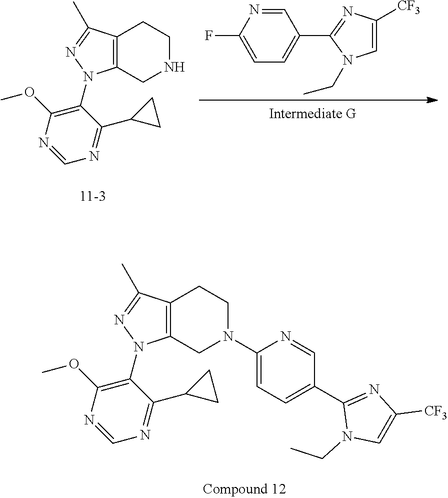

### 1-(4-cyclopropyl-6-methoxypyrimidin-5-yl)-6-(5-(1-ethyl-4-(trifluoromethyl)-1H-imidazol-2-yl)pyridin-2-yl)-3-methyl-4,5,6,7-tetrahydro-1H-pyrazolo[3,4-c]pyridine

To a solution of 1-(6-cyclopropyl-4-methoxypyrimidin-5-yl)-3-methyl-4,5,6,7-tetrahydropyrazolo[3,4-c]pyridine (50.0 mg, 0.18 mmol) and 5-[3-ethyl-5-(trifluoromethyl)imidazol-2-yl]-2-fluoropyridine (90.0 mg, 0.35 mmol) in DMSO (3 mL) was added DIEA (90.0 mg, 0.70 mmol). The mixture was stirred at 130° C. for 16 hrs. To the cooled reaction mixture was added water (20 mL) and extracted with EtOAc (20 mL×2). The combined organic layers were washed with brine (20 mL×2), dried over Na₂SO₄, filtered through a Celite® pad and the filtrate was concentrated under reduced pressure to give a residue, which was purified by pre-TLC (DCM/EA=1/1) to give the title compound.

LC-MS(ESI+): m/z 525.2 (M+H)⁺.

¹H NMR (400 MHz, DMSO-d₆) δ=8.72 (s, 1H), 8.33 (d, J=2.0 Hz, 1H), 7.98 (s, 1H), 7.80-7.88 (m, 1H), 7.08 (d, J=8.8 Hz, 1H), 4.58-4.45 (m, 2H), 4.12-4.02 (m, 2H), 3.99-3.88 (m, 5H), 2.67-2.55 (m, 2H), 2.16 (s, 3H), 1.64-1.54 (m, 1H), 1.32 (t, J=7.2 Hz, 2H), 1.15-1.10 (m, 1H), 1.05-0.97 (m, 3H).

### Example B22: Synthesis of Compound 13

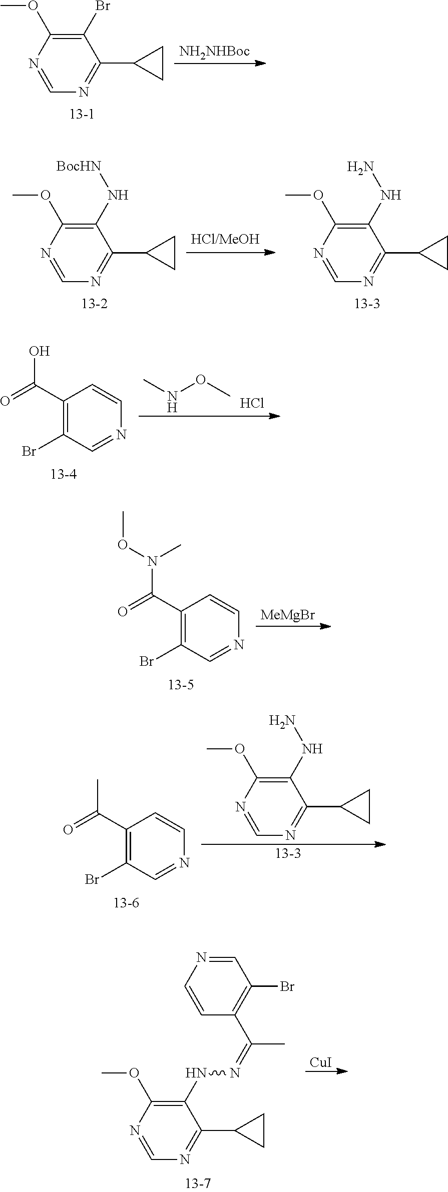

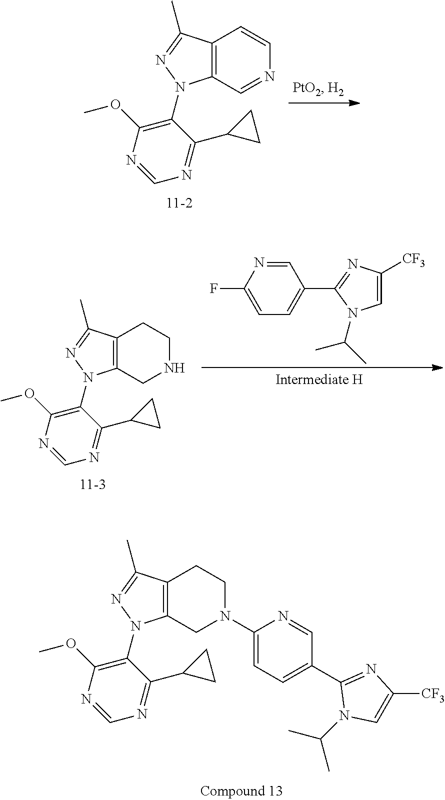 [SMILES: COc1ncnc(C2CC2)c1-n1nc(C)c2ccncc21 ; COc1ncnc(C2CC2)c1-n1nc(C)c2c1CNCC2 ; CC(C)n1cc(C(F)(F)F)nc1-c1ccc(F)nc1 ; COc1ncnc(C2CC2)c1-n1nc(C)c2c1CN(c1ccc(-c3nc(C(F)(F)F)cn3C(C)C)cn1)CC2]

### Tert-butyl 2-(4-cyclopropyl-6-methoxypyrimidin-5-yl)hydrazine-1-carboxylate

To a solution of 5-bromo-6-cyclopropyl-4-methoxypyrimidine (5.00 g, 21.82 mmol) in toluene (60 mL) were added tert-butyl hydrazinecarboxylate (5.80 g, 43.89 mmol), Pd(OAc)₂ (500.0 mg, 2.23 mmol)), X-PHOS (1.04 g, 2.18 mmol) and sodium tert-butoxide (4.20 g, 43.70 mmol). The reaction was stirred at 100° C. for 16 hrs under Ar. To the cooled reaction mixture was added water (100 mL) and extracted with EtOAc (100 mL×2). The combined organic fractions were washed with brine (100 mL), dried over Na₂SO₄, filtered through a Celite® pad and the filtrate was concentrated under reduced pressure to give a residue, which was purified by flash column chromatography on silica gel ethyl acetate (from 0% to 40%) in petroleum ether to give the title compound (3.20 g, 11.42 mmol, 52% yield) as a white solid.

LC-MS(ESI+): m/z 281.1 (M+H)⁺.

### 4-cyclopropyl-5-hydrazinyl-6-methoxypyrimidine

A solution of tert-butyl 2-(4-cyclopropyl-6-methoxypyrimidin-5-yl)hydrazine-1-carboxylate (3.20 g, 11.42 mmol) in HCl/MeOH (4 M, 20 mL) was stirred at room temperature for 3 hrs. The volatiles were removed to give the title compound (1.91 g, 10.61 mmol, 93% yield) as a yellow solid, which was used for next step directly.

LC-MS(ESI+): m/z 181.1 (M+H)⁺.

### 3-bromo-N-methoxy-N-methylisonicotinamide

To a solution of 3-bromopyridine-4-carboxylic acid (15.00 g, 74.25 mmol), EDCI (17.10 g, 89.11 mmol), HOBt (12.04 g, 89.11 mmol) and N,O-Dimethylhydroxylamine hydrochloride (10.86 g, 111.38 mmol) in DCM (150 mL) was added TEA (30.06 g, 297.03 mmol). The mixture was stirred at room temperature for 16 hrs. The reaction mixture was diluted with DCM (100 mL) and the organic phase was washed with water (2×100 mL). The combined organic layers were washed with brine (100 mL), dried over Na₂SO₄, filtered through a Celite® pad and the filtrate was concentrated under reduced pressure to give a residue, which was purified by column chromatography on silica gel eluting with ethyl acetate (from 0% to 20%) in petroleum ether to give the title compound (13.00 g, 53.04 mmol, 71% yield) as a yellow solid.

LC-MS(ESI+): m/z 244.9 (M+H)⁺.

### 1-(3-bromopyridin-4-yl)ethan-1-one

To a stirred solution of 3-bromo-N-methoxy-N-methylpyridine-4-carboxamide (13.00 g, 53.04 mmol) in dry THF (130 mL) was added methylmagnesium bromide (26.52 mL, 3 M in THF, 79.56 mmol) slowly at 0° C. and the mixture was stirred at 0° C. for 2 hrs. The reaction mixture was quenched with saturated aqueous NH₄Cl solution (60 mL) at 0° C. and the aqueous phase was extracted with EtOAc (100 mL×2). The combined organic layers were washed with brine (100 mL), dried over Na₂SO₄, filtered through a Celite® pad and the filtrate was concentrated under reduced pressure to give a residue, which was purified by column chromatography on silica gel eluting with ethyl acetate (from 0% to 12%) in petroleum ether to give the title compound (8.70 g, 43.50 mmol, 82% yield) as a colorless oil.

LC-MS(ESI+): m/z 200.9 (M+H)⁺.

### 5-(2-(1-(3-bromopyridin-4-yl)ethylidene)hydrazinyl)-4-cyclopropyl-6-methoxypyrimidine

To a solution of 1-(3-bromopyridin-4-yl)ethan-1-one (3.00 g, 14.98 mmol) in EtOH (50 mL) were added 4-cyclopropyl-5-hydrazinyl-6-methoxypyrimidine (3.00 g, 16.65 mmol) and NaOAc (3.41 g, 41.62 mmol). The mixture was stirred at 80° C. for 2 hr. To the cooled reaction mixture was added water (30 mL) and the aqueous phase was extracted with EtOAc (20 mL×2). The combined organic layers were washed with brine (50 mL), dried over Na₂SO₄, filtered through a Celite® pad and the filtrate was concentrated under reduced pressure to give a residue, which was purified by flash column chromatography on silica gel eluting with ethyl acetate (from 0% to 25%) in petroleum ether to give the title compound (3.00 g, 8.28 mmol, 55% yield) as a yellow oil.

LC-MS(ESI+): m/z 362.0 (M+H)⁺.

### 1-(4-cyclopropyl-6-methoxypyrimidin-5-yl)-3-methyl-1H-pyrazolo[3,4-c]pyridine

To a solution of 5-(2-(1-(3-bromopyridin-4-yl)ethylidene)hydrazinyl)-4-cyclopropyl-6-methoxypyrimidine (500.0 mg, 1.38 mmol) in DMSO (6 mL) were added CuI (26.0 mg, 0.14 mmol) and K₃PO₄ (586.0 mg, 2.76 mmol). The mixture was stirred at 100° C. for 16 hrs. To the cooled reaction mixture was added water (15 mL) and the aqueous phase was extracted with EtOAc (15 mL×2). The combined organic layers were washed with brine (15 mL), dried over Na₂SO₄, filtered through a Celite® pad and the filtrate was concentrated under reduced pressure to give a residue, which was purified by flash column chromatography on silica gel eluting with ethyl acetate (from 0% to 30%) in petroleum ether to give the title compound (180.0 mg, 0.64 mmol, 46% yield) as a yellow solid.

LC-MS(ESI+): m/z 282.1 (M+H)⁺.

¹H NMR (400 MHz, CDCl₃) δ=8.69 (s, 1H), 8.64 (s, 1H), 8.41 (d, J=5.6 Hz, 1H), 7.83-7.55 (m, 1H), 3.92 (s, 3H), 2.69 (s, 3H), 1.59-1.52 (m, 1H), 1.35-1.26 (m, 1H), 1.21-1.17 (m, 1H), 1.02-0.99 (m, 1H), 0.94-0.90 (m, 1H).

### 1-(4-cyclopropyl-6-methoxypyrimidin-5-yl)-3-methyl-4,5,6,7-tetrahydro-1H-pyrazolo[3,4-c]pyridine

To a solution of 1-(4-cyclopropyl-6-methoxypyrimidin-5-yl)-3-methyl-1H-pyrazolo[3,4-c]pyridine (180.0 mg, 0.64 mmol) in EtOH (5 mL) was added Platinum(IV) oxide (87.0 mg, 0.38 mmol). The reaction was stirred at room temperature for 16 hrs under H₂. The mixture was filtered through a Celite® pad and the filtrate was concentrated under reduced pressure to give the title compound (180.0 mg, 0.63 mmol, 98% yield) as a white solid, which was used for next step directly.

LC-MS(ESI+): m/z 286.1 (M+H)⁺.

### 1-(4-cyclopropyl-6-methoxypyrimidin-5-yl)-6-(5-(1-isopropyl-4-(trifluoromethyl)-1H-imidazol-2-yl)pyridin-2-yl)-3-methyl-4,5,6,7-tetrahydro-1H-pyrazolo[3,4-c]pyridine

To a solution of 1-(6-cyclopropyl-4-methoxypyrimidin-5-yl)-3-methyl-4,5,6,7-tetrahydropyrazolo[3,4-c]pyridine (48.5 mg, 0.17 mmol) and 2-fluoro-5-(1-isopropyl-4-(trifluoromethyl)-1H-imidazol-2-yl)pyridine (95.0 mg, 0.35 mmol) in DMSO (3 mL) was added DIEA (90.0 mg, 0.70 mmol). The mixture was stirred at 130° C. for 16 hrs. To the cooled reaction mixture was added water (20 mL) and the aqueous phase was extracted with EtOAc (20 mL×2). The combined organic layers were washed with brine (20 mL×2), dried over Na₂SO₄, filtered through a Celite® pad and the filtrate was concentrated under reduced pressure to give a residue, which was purified by Pre-TLC (DCM/EA=1/1) to give the title compound.

LC-MS(ESI+): m/z 539.2 (M+H)⁺.

¹H NMR (400 MHz, DMSO-d₆) δ=8.72 (s, 1H), 8.26 (d, J=2.0 Hz, 1H), 8.14 (s, 1H), 7.80-7.70 (m, 1H), 7.09 (d, J=9.2 Hz, 1H), 4.57-4.42 (m, 2H), 4.40-4.20 (m, 1H), 3.97-3.91 (m, 5H), 2.72-2.60 (m, 2H), 2.16 (s, 3H), 1.65-1.50 (m, 1H), 1.39 (d, J=6.8 Hz, 6H), 1.14-1.10 (m, 1H), 1.04-0.98 (m, 3H).

### Example B23: Synthesis of Compound 14

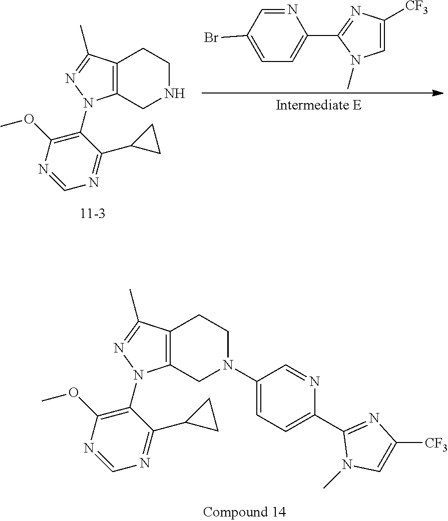 [SMILES: COc1ncnc(C2CC2)c1-n1nc(C)c2c1CNCC2 ; Cn1cc(C(F)(F)F)nc1-c1ccc(Br)cn1 ; COc1ncnc(C2CC2)c1-n1nc(C)c2c1CN(c1ccc(-c3nc(C(F)(F)F)cn3C)nc1)CC2]

### 1-(4-cyclopropyl-6-methoxypyrimidin-5-yl)-3-methyl-6-(6-(1-methyl-4-(trifluoromethyl)-1H-imidazol-2-yl)pyridin-3-yl)-4,5,6,7-tetrahydro-1H-pyrazolo[3,4-c]pyridine

To a solution of 1-(6-cyclopropyl-4-methoxypyrimidin-5-yl)-3-methyl-4,5,6,7-tetrahydropyrazolo[3,4-c]pyridine (60.0 mg, 0.21 mmol) and 5-bromo-2-[3-methyl-5-(trifluoromethyl)imidazol-2-yl]pyridine (96.0 mg, 0.31 mmol) in toluene (1 mL) was added Pd(OAc)₂ (5.0 mg, 0.02 mmol), X-PHOS (200.0 mg, 0.42 mmol) and sodium tert-butoxide (40.0 mg, 0.42 mmol). The mixture was stirred at 100° C. for 16 hrs under Ar. To the cooled reaction mixture was added water (20 mL) and the aqueous phase was extracted with EtOAc (20 mL×2). The combined organic layers were washed with brine (20 mL), dried over Na₂SO₄, filtered through a Celite® pad and the filtrate was concentrated under reduced pressure to give a residue, which was purified by pre-TLC (DCM/EA=1/1) to give the title compound.

LC-MS(ESI+): m/z 511.1 (M+H)⁺.

¹H NMR (400 MHz, DMSO-d₆) δ=8.71 (s, 1H), 8.40 (d, J=2.8 Hz, 1H), 7.95-7.80 (m, 2H), 7.8-7.48 (m, 1H), 4.33-4.22 (m, 2H), 4.02 (s, 3H), 3.94 (s, 3H), 3.85-3.70 (m, 2H), 2.70-2.54 (m, 2H), 2.15 (s, 3H), 1.56-1.52 (m, 1H), 1.20-1.10 (m, 1H), 1.08-0.96 (m, 3H).

### Example B24: Synthesis of Compound 15

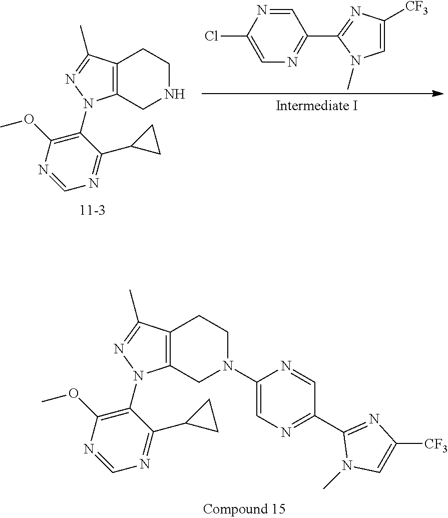 [SMILES: COc1ncnc(C2CC2)c1-n1nc(C)c2c1CNCC2 ; Cn1cc(C(F)(F)F)nc1-c1cnc(Cl)cn1 ; COc1ncnc(C2CC2)c1-n1nc(C)c2c1CN(c1cnc(-c3nc(C(F)(F)F)cn3C)cn1)CC2]

### 1-(4-cyclopropyl-6-methoxypyrimidin-5-yl)-3-methyl-6-(5-(1-methyl-4-(trifluoromethyl)-1H-imidazol-2-yl)pyrazin-2-yl)-4,5,6,7-tetrahydro-1H-pyrazolo[3,4-c]pyridine

To a solution of 1-(6-cyclopropyl-4-methoxypyrimidin-5-yl)-3-methyl-4,5,6,7-tetrahydropyrazolo[3,4-c]pyridine (60.0 mg, 0.21 mmol), 2-chloro-5-[3-methyl-5-(trifluoromethyl)imidazol-2-yl]pyrazine (82.8 mg, 0.32 mmol) in DMF (8 mL) was added DIEA (81.4 mg, 0.63 mmol). The mixture was stirred at 100° C. for 16 hrs. To the cooled reaction mixture was added water (20 mL) and the aqueous phase was extracted with EtOAc (20 mL×3). The combined organic layers were washed with brine (30 mL×2), dried over Na₂SO₄, filtered through a Celite® pad and the filtrate was concentrated under reduced pressure to give a residue, which was purified by pre-TLC (PE/EA=2/1) to give the title compound.

LC-MS(ESI+): m/z 512.4 (M+H)⁺.

¹H NMR (400 MHz, DMSO-d₆) δ=8.72 (s, 1H), 8.66 (s, 1H), 8.51 (s, 1H), 7.93 (s, 1H), 4.65-4.50 (m, 2H), 4.10-3.97 (m, 2H), 3.95 (s, 3H), 3.93 (s, 3H), 2.70-2.60 (m, 2H), 2.16 (s, 3H), 1.59-1.49 (m, 1H), 1.12-0.98 (m, 4H).

### Example B25: Synthesis of Compound 16

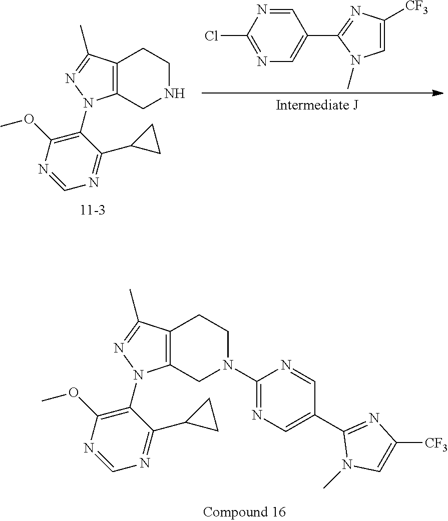 [SMILES: COc1ncnc(C2CC2)c1-n1nc(C)c2c1CNCC2 ; Cn1cc(C(F)(F)F)nc1-c1cnc(Cl)nc1 ; COc1ncnc(C2CC2)c1-n1nc(C)c2c1CN(c1ncc(-c3nc(C(F)(F)F)cn3C)cn1)CC2]

### 3-(6-cyclopropyl-4-methoxypyrimidin-5-yl)-1-methyl-5-{5-[3-methyl-5-(trifluoromethyl)imidazol-2-yl]pyrimidin-2-yl}-4,5,6,7-tetrahydropyrazolo[4,3-c]pyridine

To a solution of 2-chloro-5-[3-methyl-5-(trifluoromethyl)imidazol-2-yl]pyrimidine (55.2 mg, 0.21 mmol) and 1-(6-cyclopropyl-4-methoxypyrimidin-5-yl)-3-methyl-4,5,6,7-tetrahydropyrazolo[3,4-c]pyridine (40.0 mg, 0.14 mmol) in IPA (5 mL) was added DIEA (54.0 mg, 0.42 mmol). The mixture was stirred at 100° C. for 16 hrs. To the cooled reaction mixture was added water (100 mL) and the aqueous phase was extracted with EtOAc (10 mL×2). The combined organic layers were washed with brine (10 mL), dried over Na₂SO₄, filtered through a Celite® pad and the filtrate was concentrated under reduced pressure to give a residue, which was purified by pre-TLC (PE/EA=2/1) to give the title compound.

LC-MS(ESI+): m/z 512.3 (M+H)⁺.

¹H NMR (400 MHz, DMSO-d₆) δ=8.80-8.65 (m, 3H), 7.93 (s, 1H), 4.74-4.61 (m, 2H), 4.30-4.20 (m, 1H), 4.07-4.00 (m, 1H), 3.94 (s, 3H), 3.75 (s, 3H), 2.70-2.60 (m, 2H), 2.17 (s, 3H), 1.63-1.50 (m, 1H), 1.21-1.10 (m, 1H), 1.04-0.95 (m, 3H).

### Example B26: Synthesis of Compound 17

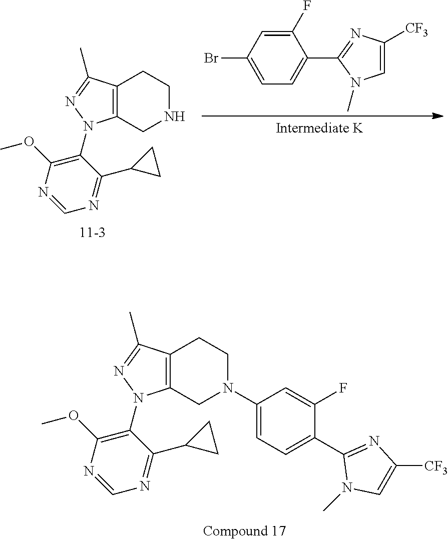 [SMILES: COc1ncnc(C2CC2)c1-n1nc(C)c2c1CNCC2 ; Cn1cc(C(F)(F)F)nc1-c1ccc(Br)cc1F ; COc1ncnc(C2CC2)c1-n1nc(C)c2c1CN(c1ccc(-c3nc(C(F)(F)F)cn3C)c(F)c1)CC2]

### 1-(4-cyclopropyl-6-methoxypyrimidin-5-yl)-6-(3-fluoro-4-(1-methyl-4-(trifluoromethyl)-1H-imidazol-2-yl)phenyl)-3-methyl-4,5,6,7-tetrahydro-1H-pyrazolo[3,4-c]pyridine

To a solution of 1-(6-cyclopropyl-4-methoxypyrimidin-5-yl)-3-methyl-4,5,6,7-tetrahydropyrazolo[3,4-c]pyridine (60.0 mg, 0.21 mmol) and 2-(4-bromo-2-fluorophenyl)-3-methyl-5-(trifluoromethyl)imidazole (101.9 mg, 0.32 mmol) in toluene (3 mL) was added Pd(OAc)₂ (4.7 mg, 0.02 mmol), X-PHOS (200.4 mg, 0.42 mmol) and sodium tert-butoxide (40.4 mg, 0.42 mmol) at room temperature. The mixture was stirred at 100° C. for 16 hrs under Ar. To the cooled reaction mixture was added water (20 mL) and the aqueous phase was extracted with EtOAc (20 mL×2). The combined organic layers were washed with brine (15 mL), dried over Na₂SO₄, filtered through a Celite® pad and the filtrate was concentrated under reduced pressure to give a residue, which was purified by pre-TLC (DCM/EtOAc=1/1) to give the title compound.

LC-MS(ESI+): m/z 528.1 (M+H)⁺.

¹H NMR (400 MHz, DMSO-d₆) δ=8.71 (s, 1H), 7.92 (s, 1H), 7.42-7.29 (m, 1H), 7.05-6.90 (m, 1H), 4.29-4.18 (m, 2H), 3.93 (s, 3H), 3.82-3.69 (m, 2H), 3.56 (s, 3H), 2.75-2.65 (m, 2H), 2.15 (s, 3H), 1.60-1.47 (m, 1H), 1.21-0.96 (m, 4H).

### Example B27: Synthesis of Compound 18

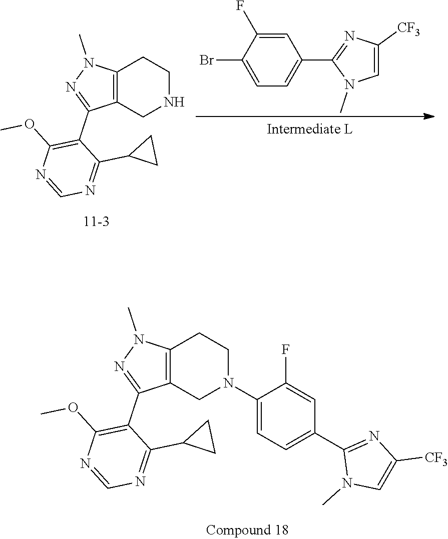

### 1-(6-cyclopropyl-4-methoxypyrimidin-5-yl)-6-{2-fluoro-4-[3-methyl-5-(trifluoromethyl)imidazol-2-yl]phenyl}-3-methyl-4,5,6,7-tetrahydropyrazolo[3,4-c]pyridine

To a solution of 1-(6-cyclopropyl-4-methoxypyrimidin-5-yl)-3-methyl-4,5,6,7-tetrahydropyrazolo[3,4-c]pyridine (50.0 mg, 0.18 mmol) and 2-(4-bromo-3-fluorophenyl)-3-methyl-5-(trifluoromethyl)imidazole (113.2 mg, 0.35 mmol) in toluene (2 mL) was added Pd(OAc)₂ (3.9 mg, 0.02 mmol), X-PHOS (167.0 mg, 0.35 mmol) and sodium tert-butoxide (33.7 mg, 0.35 mmol) at room temperature. The mixture was stirred at 100° C. for 16 hrs under Ar. To the cooled reaction mixture was added water (10 mL) and the aqueous phase was extracted with EtOAc (10 mL×2). The combined organic layers were washed with brine (20 mL), dried over Na₂SO₄, filtered through a Celite® pad and the filtrate was and concentrated under reduced pressure to give a residue, which was purified by pre-TLC (DCM/EA=1/1) to give the title compound.

¹H NMR (400 MHz, DMSO-d₆) δ=8.69 (s, 1H), 7.91 (s, 1H), 7.53-7.49 (m, 1H), 7.43-7.40 (m, 1H), 7.20-7.08 (m, 1H), 4.15-4.02 (m, 2H), 3.94 (s, 3H), 3.77 (s, 3H), 3.55-3.47 (m, 2H), 2.64-2.59 (m, 2H), 2.17 (s, 3H), 1.63-1.55 (m, 1H), 1.17-1.05 (m, 1H), 1.01-0.90 (m, 3H).

### Example B28: Synthesis of Compound 19

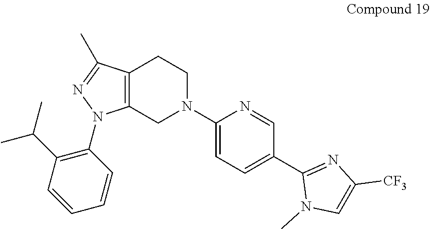 [SMILES: Cc1nn(-c2ccccc2C(C)C)c2c1CCN(c1ccc(-c3nc(C(F)(F)F)cn3C)cn1)C2]

The synthetic route for Compound19 was similar to that of Compound 11 by replacing 11-1 with (2-isopropylphenyl)boronic acid. The crude product was purified by pre-TLC (PE/EA=1/1) to give the title compound.

LC-MS(ESI+): m/z 481.1 (M+H)⁺.

¹H NMR (400 MHz, DMSO-d₆) δ=8.37 (d, J=2.0 Hz, 1H), 7.98-7.80 (m, 2H), 7.59-7.51 (m, 2H), 7.38-7.34 (m, 1H), 7.32-7.21 (m, 1H), 7.06 (d, J=9.2 Hz, 1H), 4.54 (s, 2 H), 3.98-3.88 (m, 2H), 3.73 (s, 3H), 2.75-2.68 (m, 1H), 2.65-2.52 (m, 2H), 2.15 (s, 3H), 1.09 (d, J=6.8 Hz, 6H).

### Example B29: Synthesis of Compound 20

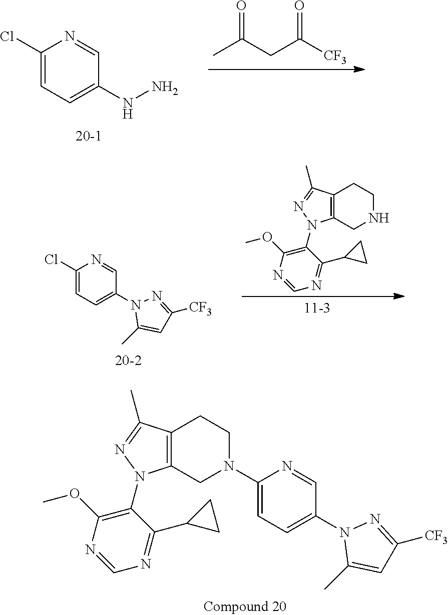

### 2-chloro-5-(5-methyl-3-(trifluoromethyl)-1H-pyrazol-1-yl)pyridine

To a solution of 2-chloro-5-hydrazinylpyridine (0.90 g, 6.26 mmol) in HFIP (10 mL) was added TEA (1.90 g, 18.80 mmol) and 1,1,1-trifluoropentane-2,4-dione (965.8 mg, 6.26 mmol) slowly at 0° C. The mixture was stirred at 0° C. for 2 hrs and stirred at room temperature for 16 hrs. To the reaction mixture was added water (50 mL) and the aqueous phase was extracted with EtOAc (50 mL×2). The combined organic layers were washed with brine (50 mL), dried over Na₂SO₄, filtered through a Celite® pad and the filtrate was concentrated under reduced pressure to give a residue, which was purified by flash column chromatography on silica gel eluting with ethyl acetate (from 0% to 10%) in petroleum ether to give the title compound (950.0 mg, 3.63 mmol, 58% yield) as white solid.

LC-MS(ESI+): m/z 262.0 (M+H)⁺.

### 1-(4-cyclopropyl-6-methoxypyrimidin-5-yl)-3-methyl-6-(5-(5-methyl-3-(trifluoromethyl)-1H-pyrazol-1-yl)pyridin-2-yl)-4,5,6,7-tetrahydro-1H-pyrazolo[3,4-c]pyridine

To a solution of 1-(4-cyclopropyl-6-methoxypyrimidin-5-yl)-3-methyl-4,5,6,7-tetrahydro-1H-pyrazolo[3,4-c]pyridine (60.0 mg, 0.21 mmol) and 2-chloro-5-(5-methyl-3-(trifluoromethyl)-1H-pyrazol-1-yl)pyridine (82.5 mg, 0.31 mmol) in toluene (2 mL) was added Pd(OAc)₂ (4.7 mg, 0.02 mmol), X-PHOS (200.4 mg, 0.42 mmol) and sodium tert-butoxide (40.4 mg, 0.42 mmol). The mixture was stirred at 100° C. for 16 hrs under Ar. To the cooled reaction mixture was added water (20 mL) and the aqueous phase was extracted with EtOAc (20 mL×3). The combined organic layers were washed with brine (30 mL×2), dried over Na₂SO₄, filtered through a Celite® pad and the filtrate was concentrated under reduced pressure to give a residue, which was purified by Prep-TLC twice (PE/EA=1/1, DCM/EA=3/1) to give the title compound.

LC-MS (ESI+): m/z 511.1 (M+H)⁺.

¹H NMR (400 MHz, DMSO-d₆) δ 8.71 (s, 1H), 8.26 (d, J=2.8 Hz, 1H), 7.80-7.70 (m, 1H), 7.10 (d, J=9.2 Hz, 1H), 6.72 (s, 1H), 4.60-4.40 (m, 2H), 3.99-3.87 (m, 5H), 2.70-2.58 (m, 2H), 2.27 (s, 3H), 2.15 (s, 3H), 1.65-1.52 (m, 1H), 1.14-0.94 (m, 4H).

### Example B30: Synthesis of Compound 21

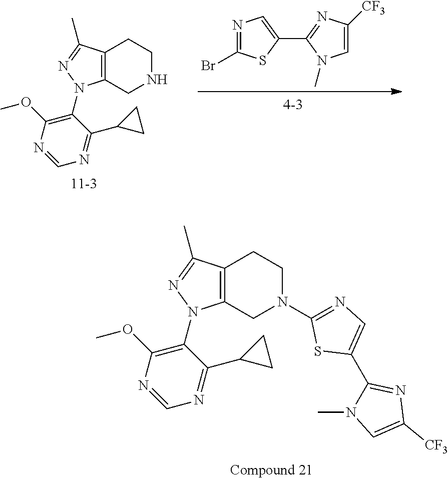 [SMILES: COc1ncnc(C2CC2)c1-n1nc(C)c2c1CNCC2 ; Cn1cc(C(F)(F)F)nc1-c1cnc(Br)s1 ; COc1ncnc(C2CC2)c1-n1nc(C)c2c1CN(c1ncc(-c3nc(C(F)(F)F)cn3C)s1)CC2]

### 2-(1-(4-cyclopropyl-6-methoxypyrimidin-5-yl)-3-methyl-1,4,5,7-tetrahydro-6H-pyrazolo[3,4-c]pyridin-6-yl)-5-(1-methyl-4-(trifluoromethyl)-1H-imidazol-2-yl)thiazole

To a solution of 1-(4-cyclopropyl-6-methoxypyrimidin-5-yl)-3-methyl-4,5,6,7-tetrahydro-1H-pyrazolo[3,4-c]pyridine (50.0 mg, 0.18 mmol) and 2-bromo-5-(1-methyl-4-(trifluoromethyl)-1H-imidazol-2-yl)thiazole (109.0 mg, 0.35 mmol) in DMSO (3 mL) was added DIEA (90.0 mg, 0.70 mmol). The reaction was stirred at 90° C. for 16 hrs. To the cooled reaction mixture was added water (20 mL) and the aqueous phase was extracted with EtOAc (20 mL×2). The combined organic layers were washed with brine (20 mL×2), dried over Na₂SO₄, filtered through a Celite® pad and the filtrate was concentrated under reduced pressure to give a residue, which was purified by Pre-TLC (PE/EA=1/1) to give the title compound.

LC-MS(ESI+): m/z 517.1 (M+H)⁺.

¹H NMR (400 MHz, DMSO-d₆) δ=8.71 (s, 1H), 7.85 (s, 1H), 7.61 (s, 1H), 4.50-4.40 (s, 2H), 3.94 (s, 3H), 3.86-3.78 (m, 5H), 2.75-2.60 (m, 2H), 2.17 (s, 3H), 1.70-1.53 (m, 1H), 1.20-1.07 (m, 1H), 1.03-0.95 (m, 3H).

### Example B31: Synthesis of Compound 22

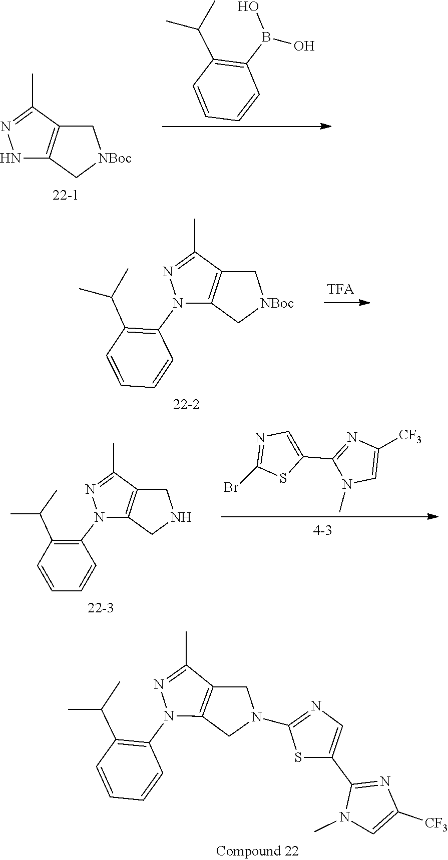 [SMILES: Cc1n[nH]c2c1CN(C(=O)OC(C)(C)C)C2 ; CC(C)c1ccccc1B(O)O ; Cc1nn(-c2ccccc2C(C)C)c2c1CN(C(=O)OC(C)(C)C)C2 ; Cc1nn(-c2ccccc2C(C)C)c2c1CNC2 ; Cn1cc(C(F)(F)F)nc1-c1cnc(Br)s1 ; Cc1nn(-c2ccccc2C(C)C)c2c1CN(c1ncc(-c3nc(C(F)(F)F)cn3C)s1)C2]

### Tert-butyl 1-(2-isopropylphenyl)-3-methyl-4,6-dihydropyrrolo[3,4-c]pyrazole-5(1H)-carboxylate

To a solution of tert-butyl 3-methyl-4,6-dihydropyrrolo[3,4-c]pyrazole-5(1H)-carboxylate (0.20 g, 0.89 mmol), (2-isopropylphenyl)boronic acid (294.0 mg, 1.79 mmol) in DCE (6 mL) was added copper (II) acetate (49.0 mg, 0.27 mmol) and pyridine (142.0 mg, 1.79 mmol). The reaction was stirred 70° C. for 16 hrs. To the cooled reaction mixture was added water (20 mL) and the aqueous phase was extracted with DCM (20 mL×2). The combined organic layers were washed with brine (20 mL), dried over Na₂SO₄, filtered through a Celite® pad and the filtrate was concentrated under reduced pressure to give a residue, which was purified by Pre-TLC (PE/EA=5/1) to give the title compound (100.0 mg, 0.29 mmol, 33% yield) as a yellow solid.

LC-MS(ESI+): m/z 342.2 (M+H)⁺.

### 1-(2-isopropylphenyl)-3-methyl-1,4,5,6-tetrahydropyrrolo[3,4-c]pyrazole

To a solution of tert-butyl 1-(2-isopropylphenyl)-3-methyl-4,6-dihydropyrrolo[3,4-c]pyrazole-5(1H)-carboxylate (80.0 mg, 0.23 mmol) in DCM (2 mL) was added TFA (1 mL) at 0° C. The reaction was stirred at room temperature for 1 hr. The mixture was concentrated to give the title compound (50.0 mg, 0.21 mmol, 91% yield) as a yellow solid, which was used for next step directly.

LC-MS(ESI+): m/z 242.2 (M+H)⁺.

### 2-(1-(2-isopropylphenyl)-3-methyl-4,6-dihydropyrrolo[3,4-c]pyrazol-5(1H)-yl)-5-(1-methyl-4-(trifluoromethyl)-1H-imidazol-2-yl)thiazole

To a solution of 1-(2-isopropylphenyl)-3-methyl-1,4,5,6-tetrahydropyrrolo[3,4-c]pyrazole (35.0 mg, 0.14 mmol) and 2-bromo-5-(1-methyl-4-(trifluoromethyl)-1H-imidazol-2-yl)thiazole (68.0 mg, 0.22 mmol) in DMSO (2 mL) was added DIEA (94.0 mg, 0.73 mmol). The reaction was stirred at 90° C. for 18 hrs. To the cooled reaction mixture was added water (20 mL) and the aqueous phase was extracted with EtOAc (20 mL×2). The combined organic layers were washed with brine (20 mL×2), dried over Na₂SO₄, filtered through a Celite® pad and the filtrate was concentrated under reduced pressure to give a residue, which was purified by pre-TLC (DCM/EA=1/1) to give the title compound.

LC-MS(ESI+): m/z 473.1 (M+H)⁺.

¹H NMR (400 MHz, DMSO-d₆) δ=7.85 (s, 1H), 7.68 (s, 1H), 7.60-7.50 (m, 1H), 7.49-7.40 (m, 1H), 7.39-7.30 (m, 2H), 4.70-4.50 (m, 4H), 3.83 (s, 3H), 3.11-3.04 (m, 1H), 2.26 (s, 3H), 1.15 (d, J=7.2 Hz, 6H).

### Example B32: Synthesis of Compound 23

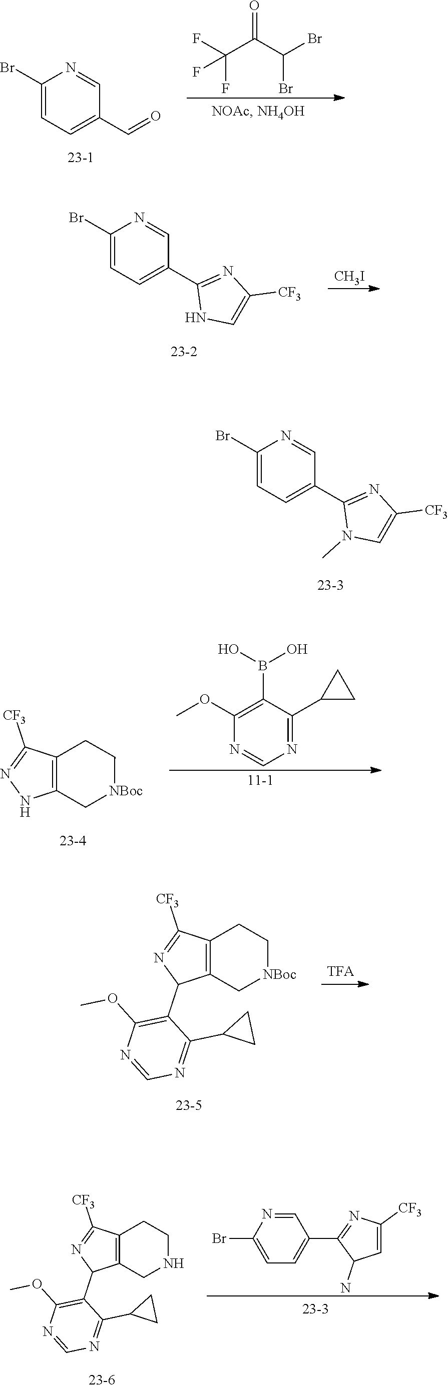

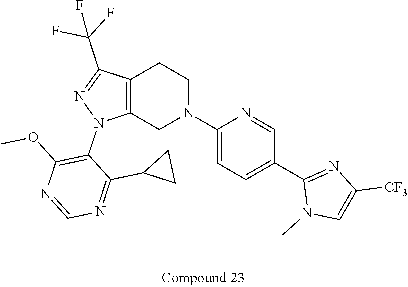 [SMILES: COc1ncnc(C2CC2)c1-n1nc(C(F)(F)F)c2c1CN(c1ccc(-c3nc(C(F)(F)F)cn3C)cn1)CC2]

### 2-bromo-5-(4-(trifluoromethyl)-1H-imidazol-2-yl)pyridine

To a solution of sodium acetate (7.94 g, 96.77 mmol) in water (120 mL) was added 3,3-dibromo-1,1,1-trifluoropropan-2-one (19.59 g, 72.58 mmol) dropwise at 0° C. Then, the mixture was stirred for 1 h at 100° C. After cooling to room temperature, a mixture of 6-bromonicotinaldehyde (9.05 g, 48.66 mmol), MeOH (240 mL) and ammonium hydroxide (28%, 80 mL) was added to the solution and the resulting mixture was stirred for 16 hrs at room temperature. The volatiles were removed in vacuo. The residue was diluted with water (150 mL) and the aqueous phase was extracted with EtOAc (150 mL×3). The combined organic fractions were washed with brine (100 mL), dried over Na₂SO₄, filtered through a Celite® pad and the filtrate was concentrated under reduced pressure to give a residue, which was purified by flash column chromatography on silica gel eluting with ethyl acetate (from 0% to 20%) in petroleum ether to give the title compound (9.00 g, 30.81 mmol, 63% yield) as a yellow solid.

LC-MS(ESI+): m/z 291.9 (M+H)⁺.

¹H NMR (400 MHz, DMSO-d₆) δ=13.50 (br.s, 1H), 8.94 (d, J=2.4 Hz, 1H), 8.25 (dd, J=8.4 Hz, J=2.4 Hz, 1H), 8.05 (s, 1H), 7.80 (d, J=8.4 Hz, 1H).

### 2-bromo-5-(1-methyl-4-(trifluoromethyl)-1H-imidazol-2-yl)pyridine

To a solution of 2-bromo-5-(4-(trifluoromethyl)-1H-imidazol-2-yl)pyridine (5.02 g, 17.18 mmol) and Cs₂CO₃ (13.94 g, 42.79 mmol) in DMF (60 mL) was added iodomethane (4.86 g, 34.23 mmol) dropwise at 0° C. The mixture was gradually warmed to room temperature and stirred at room temperature for 16 hrs. To the reaction mixture was added water (100 mL) and the aqueous phase was extracted with EtOAc (100 mL×2). The combined organic layers were washed with brine (150 mL), dried over Na₂SO₄, filtered through a Celite® pad and the filtrate was concentrated under reduced pressure to give a residue, which was purified by flash column chromatography on silica gel eluting with ethyl acetate (from 0% to 20%) in petroleum ether to give the title compound (2.9 g, 9.50 mmol, 55% yield) as a yellow solid.

LC-MS(ESI+): m/z 305.9 (M+H)⁺.

¹H NMR (400 MHz, DMSO-d₆) δ=8.75 (d, J=2.4 Hz, 1H), 8.11 (dd, J=8.4 Hz, 2.4 Hz, 1H), 8.04 (s, 1H), 7.82 (d, J=8.0 Hz, 1H), 3.83 (s, 3H).

### Tert-butyl 1-(4-cyclopropyl-6-methoxypyrimidin-5-yl)-3-(trifluoromethyl)-1,4,5,7-tetrahydro-6H-pyrazolo[3,4-c]pyridine-6-carboxylate

To a solution of tert-butyl 3-(trifluoromethyl)-1,4,5,7-tetrahydro-6H-pyrazolo[3,4-c]pyridine-6-carboxylate (700.0 mg, 2.40 mmol) and (4-cyclopropyl-6-methoxypyrimidin-5-yl)boronic acid (699.0 mg, 3.60 mmol) in dioxane (10 mL) was added copper (II) acetate (131.0 mg, 0.72 mmol) and DBU (731.0 mg, 4.80 mmol). The mixture was stirred at 30° C. for 16 hrs. To the cooled reaction mixture was added water (20 mL) and the aqueous phase was extracted with EtOAc (20 mL×2). The combined organic layers were washed with brine (20 mL), dried over Na₂SO₄, filtered through a Celite® pad and the filtrate was concentrated under reduced pressure to give a residue, which was purified by flash column chromatography on silica gel eluting with ethyl acetate (from 0% to 5%) in petroleum ether and re-purified with pre-TLC (PE/EA=5/1) to give the title compound (100 mg, 0.23 mmol, 10% yield) as a colorless oil.

LC-MS(ESI+): m/z 440.1 (M+H)⁺.

¹H NMR (400 MHz, CDCl₃) δ=8.62 (s, 1H), 4.38-4.24 (m, 2H), 3.98 (s, 3H), 3.83-3.73 (m, 1H), 3.70-3.55 (m, 1H), 2.85-2.70 (m, 2H), 1.47 (s, 9H), 1.41-1.23 (m, 2H), 1.21-1.05 (m, 1H), 1.05-0.90 (m, 21H).

### 1-(4-cyclopropyl-6-methoxypyrimidin-5-yl)-3-(trifluoromethyl)-4,5,6,7-tetrahydro-1H-pyrazolo[3,4-c]pyridine

To a solution of tert-butyl 1-(4-cyclopropyl-6-methoxypyrimidin-5-yl)-3-(trifluoromethyl)-1,4,5,7-tetrahydro-6H-pyrazolo[3,4-c]pyridine-6-carboxylate (100 mg, 0.23 mmol) in DCM (2 mL) was added TFA (1 mL) at 0° C. The mixture was stirred at room temperature for 2 hrs. The mixture was concentrated to give the title compound (75.0 mg, 0.22 mmol, 96% yield) as a colorless oil, which was used for next step directly.

LC-MS(ESI+): m/z 340.1 (M+H)⁺.

### 1-(4-cyclopropyl-6-methoxypyrimidin-5-yl)-6-(5-(1-methyl-4-(trifluoromethyl)-1H-imidazol-2-yl)pyridin-2-yl)-3-(trifluoromethyl)-4,5,6,7-tetrahydro-1H-pyrazolo[3,4-c]pyridine

To a solution of 1-(4-cyclopropyl-6-methoxypyrimidin-5-yl)-3-(trifluoromethyl)-4,5,6,7-tetrahydro-1H-pyrazolo[3,4-c]pyridine (65.0 mg, 0.19 mmol) and 2-bromo-5-(1-methyl-4-(trifluoromethyl)-1H-imidazol-2-yl)pyridine (88.0 mg, 0.29 mmol) in toluene (2 mL) was added sodium tert-butoxide (36.5 mg, 0.38 mmol), Pd₂(dba)₃ (18.0 mg, 0.02 mmol) and BINAP (62.0 mg, 0.10 mmol). The mixture was stirred at 90° C. for 16 hrs under Ar. To the cooled reaction mixture was added water (15 mL) and the aqueous phase was extracted with EtOAc (10 mL×2). The combined organic layers were washed with brine (15 mL), dried over Na₂SO₄, filtered through a Celite® pad and the filtrate was concentrated under reduced pressure to give a residue, which was purified by pre-TLC (DCM/EA=5/1) to give the title compound.

LC-MS(ESI+): m/z 565.0 (M+H)⁺.

¹H NMR (400 MHz, DMSO-d₆) δ=8.80 (s, 1H), 8.44 (d, J=2.0 Hz, 1H), 7.95-7.80 (m, 2H), 7.13 (d, J=8.8 Hz, 1H), 4.75-4.54 (m, 2H), 4.10-3.94 (m, 5H), 3.75 (s, 3H), 2.90-2.73 (m, 2H), 1.53-1.40 (m, 1H), 1.25-1.13 (m, 1H), 1.13-1.00 (m, 3H).

### Example B33: Synthesis of Compound 24

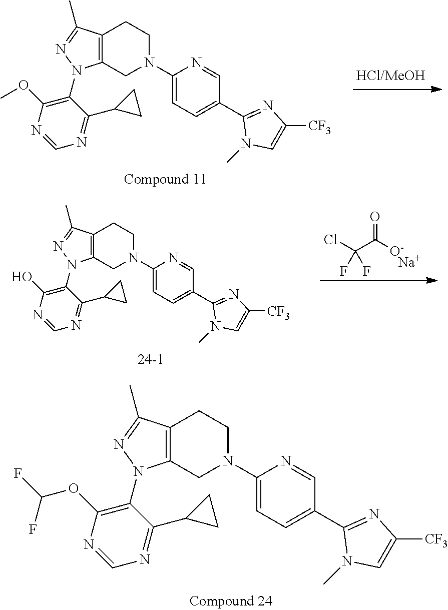 [SMILES: COc1ncnc(C2CC2)c1-n1nc(C)c2c1CN(c1ccc(-c3nc(C(F)(F)F)cn3C)cn1)CC2 ; Cc1nn(-c2c(O)ncnc2C2CC2)c2c1CCN(c1ccc(-c3nc(C(F)(F)F)cn3C)cn1)C2 ; O=C([O-])C(F)(F)Cl ; [Na+] ; Cc1nn(-c2c(OC(F)F)ncnc2C2CC2)c2c1CCN(c1ccc(-c3nc(C(F)(F)F)cn3C)cn1)C2]

### 6-cyclopropyl-5-(3-methyl-6-(5-(1-methyl-4-(trifluoromethyl)-1H-imidazol-2-yl)pyridin-2-yl)-4,5,6,7-tetrahydro-1H-pyrazolo[3,4-c]pyridin-1-yl)pyrimidin-4-ol

A solution of 1-(4-cyclopropyl-6-methoxypyrimidin-5-yl)-3-methyl-6-(5-(1-methyl-4-(trifluoromethyl)-1H-imidazol-2-yl)pyridin-2-yl)-4,5,6,7-tetrahydro-1H-pyrazolo[3,4-c]pyridine (240.0 mg, 0.47 mmol) in HCl/MeOH (10 mL, 4 M) was stirred at 100° C. for 16 hrs. After cooling to room temperature, the volatiles were removed in vacuo. To the residue was added DCM (50 mL) and washed with saturated NaHCO₃ aqueous solution (30 mL×3). The organic layer was dried over Na₂SO₄, filtered through a Celite® pad and the filtrate was concentrated under reduced pressure to give a residue, which was purified by pre-TLC (DCM/MeOH=20/1) to give the title compound (100 mg, 0.20 mmol, 42% yield) as a yellow solid.

LC-MS(ESI+): m/z 497.1 (M+H)⁺.

¹H NMR (400 MHz, DMSO-d₆) δ=12.76 (br.s, 1H), 8.41 (d, J=2.0 Hz, 1H), 8.21 (s, 1H), 7.95-7.83 (m, 2H), 7.05 (d, J=8.8 Hz, 1H), 4.62-4.43 (m, 2H), 3.99-3.85 (m, 2H), 3.74 (s, 3H), 2.65-2.55 (m, 2H), 2.13 (s, 3H), 1.53-1.46 (m, 1H), 1.01-0.84 (m, 4H).

### 1-(4-cyclopropyl-6-(difluoromethoxy)pyrimidin-5-yl)-3-methyl-6-(5-(1-methyl-4-(trifluoromethyl)-1H-imidazol-2-yl)pyridin-2-yl)-4,5,6,7-tetrahydro-1H-pyrazolo[3,4-c]pyridine

To a solution of 6-cyclopropyl-5-(3-methyl-6-(5-(1-methyl-4-(trifluoromethyl)-1H-imidazol-2-yl)pyridin-2-yl)-4,5,6,7-tetrahydro-1H-pyrazolo[3,4-c]pyridin-1-yl)pyrimidin-4-ol (40.0 mg, 0.08 mmol), sodium 2-chloro-2,2-difluoroacetate (18.0 mg, 0.12 mmol) in ACN (0.8 mL) was added Na₂CO₃ (17.0 mg, 0.16 mmol). The mixture was stirred at 90° C. for 16 hrs. To the cooled reaction mixture was added water (20 mL) and the aqueous phase was extracted with EtOAc (20 mL×3). The combined organic layers were washed with brine (30 mL×2), dried over Na₂SO₄, filtered through a Celite® pad and the filtrate was concentrated under reduced pressure to give a residue, which was purified by pre-TLC (DCM/MeOH=15/1) to give the title compound.

LC-MS(ESI+): m/z 547.1 (M+H)⁺.

¹H NMR (400 MHz, DMSO-d₆) δ=8.85 (s, 1H), 8.40 (d, J=2.4 Hz, 1H), 8.01-7.65 (m, 3H), 7.08 (d, J=8.8 Hz, 1H), 4.71-4.47 (m, 2H), 4.01-3.89 (m, 2H), 3.74 (s, 3H), 2.73-2.55 (m, 2H), 2.18 (s, 3H), 1.64-1.51 (m, 1H), 1.21-1.01 (m, 4H).

### Example B34: Synthesis of Compound 25

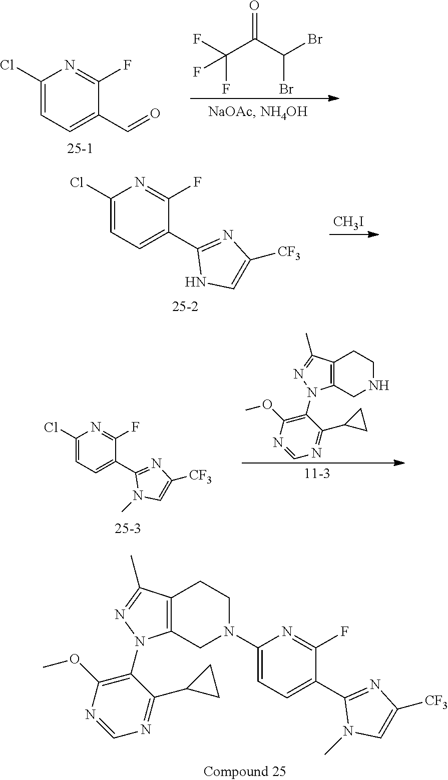

### 6-chloro-2-fluoro-3-(4-(trifluoromethyl)-1H-imidazol-2-yl)pyridine

To a solution of sodium acetate (925.0 mg, 11.28 mmol) in water (12 mL) was added 3,3-dibromo-1,1,1-trifluoropropan-2-one (2283.0 mg, 8.46 mmol) dropwise at 0° C. Then, the mixture was stirred for 1 hr at 100° C. After cooling to room temperature, a mixture of 6-chloro-2-fluoronicotinaldehyde (903.0 mg, 5.66 mmol), MeOH (24 mL), and ammonium hydroxide (28%, 8 mL) was added to the solution and the resulting mixture was stirred for 16 hrs at room temperature. The volatiles were removed in vacuo. To the residue was added water (50 mL) and the aqueous phase was extracted with EtOAc (50 mL×3). The combined organic layers were washed with brine (100 mL), dried over Na₂SO₄, filtered through a Celite® pad and the filtrate was concentrated under reduced pressure to give a residue, which was purified by flash column chromatography on silica gel eluting with ethyl acetate (from 0% to 10%) in petroleum ether to give the title compound (855 mg, 3.22 mmol, 57% yield) as a yellow solid.

LC-MS(ESI+): m/z 266.0 (M+H)⁺.

¹H NMR (400 MHz, DMSO-d₆) δ=13.19 (br.s, 1H), 8.60-8.50 (m, 1H), 8.00 (s, 1H), 7.66 (d, J=8.0 Hz, 1H).

### 6-chloro-2-fluoro-3-(1-methyl-4-(trifluoromethyl)-1H-imidazol-2-yl)pyridine

To a solution of 6-chloro-2-fluoro-3-(4-(trifluoromethyl)-1H-imidazol-2-yl)pyridine (500.0 mg, 1.88 mmol) and Cs₂CO₃ (1533.0 mg, 4.70 mmol) in DMF (6 mL) was added iodomethane (534 mg, 3.76 mmol) dropwise at 0° C. The mixture was gradually warmed to room temperature and stirred at room temperature for 16 hrs. To the reaction mixture was added water (50 mL) and the aqueous phase was extracted with EtOAc (50 mL×2). The combined organic layers were washed with brine (100 mL), dried over Na₂SO₄, filtered through a Celite® pad and the filtrate was concentrated under reduced pressure to give a residue, which was purified by flash column chromatography on silica gel eluting with ethyl acetate (from 0% to 10%) in petroleum ether to give the title compound (300.0 mg, 1.07 mmol, 57% yield) as a yellow solid.

LC-MS(ESI+): m/z 280.0 (M+H)⁺.

¹H NMR (400 MHz, DMSO-d₆) δ=8.35-8.20 (m, 1H), 8.08 (s, 1H), 7.80-7.65 (m, 1H), 3.68 (s, 3H).

### 1-(4-cyclopropyl-6-methoxypyrimidin-5-yl)-6-(6-fluoro-5-(1-methyl-4-(trifluoromethyl)-1H-imidazol-2-yl)pyridin-2-yl)-3-methyl-4,5,6,7-tetrahydro-1H-pyrazolo[3,4-c]pyridine

To a solution of 1-(4-cyclopropyl-6-methoxypyrimidin-5-yl)-3-methyl-4,5,6,7-tetrahydro-1H-pyrazolo[3,4-c]pyridine (60.0 mg, 0.21 mmol) and 6-chloro-2-fluoro-3-(1-methyl-4-(trifluoromethyl)-1H-imidazol-2-yl)pyridine (88.0 mg, 0.31 mmol) in toluene (2 mL) was added Pd(OAc)₂ (4.0 mg, 0.02 mmol), X-PHOS (200 mg, 0.42 mmol) and sodium tert-butoxide (40 mg, 0.42 mmol) under Ar. The mixture was stirred at 100° C. for 16 hrs. To the cooled reaction mixture was added water (20 mL) and the aqueous phase was extracted with EtOAc (20 mL×3). The combined organic layers were washed with brine (30 mL×2), dried over Na₂SO₄, filtered through a Celite® pad and the filtrate was concentrated under reduced pressure to give a residue, which was purified by prep-TLC twice (PE/EA=1/1, DCM/EA=3/1) to give the title compound.

LC-MS(ESI+): m/z 529.0 (M+H)⁺.

¹H NMR (400 MHz, DMSO-d₆) δ=8.72 (s, 1H), 7.94 (s, 1H), 7.90-7.78 (m, 1H), 6.98 (d, J=7.6 Hz, 1H), 4.69-4.40 (m, 2H), 3.99-3.85 (m, 5H), 3.58 (s, 3H), 2.71-2.59 (m, 2H), 2.16 (s, 3H), 1.61-1.48 (m, 1H), 1.21-0.85 (m, 4H).

### Example B35: Synthesis of Compound 26

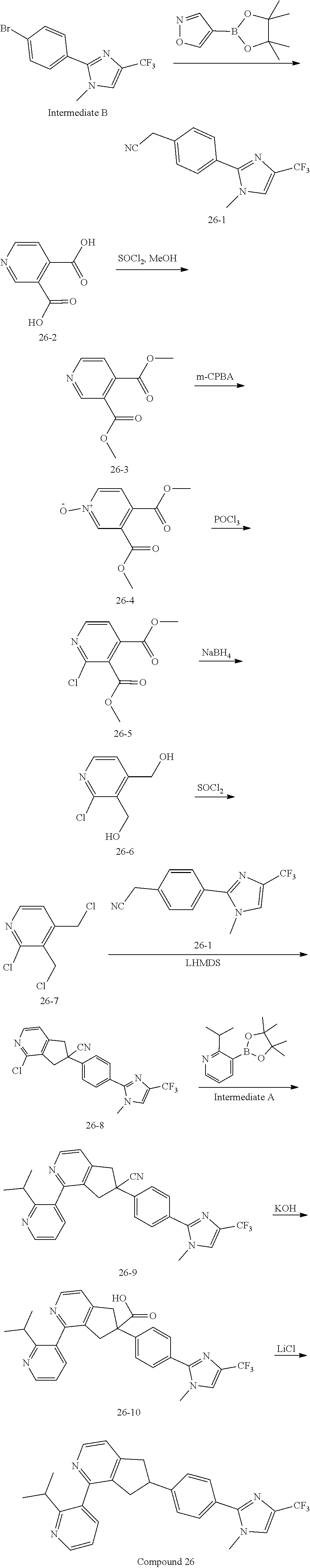

### 2-(4-(1-methyl-4-(trifluoromethyl)-1H-imidazol-2-yl)phenyl)acetonitrile

To a solution of 2-(4-bromophenyl)-1-methyl-4-(trifluoromethyl)-1H-imidazole (3.00 g, 9.83 mmol) and 4-(4,4,5,5-tetramethyl-1,3,2-dioxaborolan-2-yl)isoxazole (2.88 g, 14.75 mmol) in dioxane (30 mL) and H₂O (5 mL) were added Pd(dppf)Cl₂ (732.0 mg, 1.0 mmol) and K₂CO₃ (2.72 g, 19.66 mmol). The mixture was stirred at 100° C. for 16 hrs under N₂. To the cooled reaction mixture was added water (100 mL) and extracted with EtOAc (50 mL×3). The combined organic layers were washed with brine (100 mL), dried over Na₂SO₄, filtered through a Celite® pad and the filtrate was concentrated under reduced pressure to give a residue, which was purified by column chromatography on silica gel eluting with ethyl acetate (from 0% to 30%) in petroleum ether to give the title compound (1.20 g, 4.52 mmol, 46% yield) as a yellow solid.

LC-MS(ESI+): m/z 266.1 (M+H)⁺.

¹H NMR (400 MHz, CDCl₃) δ=7.67 (d, J=8.4 Hz, 2H), 7.46 (d, J=8.4 Hz, 2H), 7.40-7.30 (m, 1H), 3.83 (s, 2H), 3.78 (s, 3H).

### Dimethyl pyridine-3,4-dicarboxylate

To a solution of pyridine-3,4-dicarboxylic acid (20.00 g, 119.67 mmol) in SOCl₂ (200 mL) was added DMF (2 mL) at 0° C. The mixture was stirred at 80° C. for 6 hrs. The volatiles were removed in vacuo. The residue was dissolved in MeOH (100 mL) and the mixture was stirred at room temperature for 16 hrs. The solvent was removed under reduced pressure. The crude product was dissolved in EtOAc (200 mL), and it was washed with saturated NaHCO₃ aqueous solution (100 mL). The organic layers were washed with brine (80 mL), dried over Na₂SO₄, filtered through a Celite® pad and the filtrate was concentrated under reduced pressure to give the title compound (21.00 g, 107.60 mmol, 90% yield) as a brown oil, which was used for next step directly.

LC-MS(ESI+): m/z 196.0 (M+H)⁺.

¹H NMR (400 MHz, DMSO-d₆) δ=9.03 (s, 1H), 8.92 (d, J=4.8 Hz, 1H), 7.80-7.65 (m, 1H), 3.88 (s, 6H).

### 3,4-bis(methoxycarbonyl)pyridine 1-oxide

To a solution of dimethyl pyridine-3,4-dicarboxylate (11.00 g, 56.36 mmol) in DCM (120 mL) was added m-CPBA (17.16 g, 84.54 mmol, 85%) in portions over 5 minutes at 0° C. Then, the reaction was stirred at room temperature for 5 hrs. The mixture was diluted with DCM (200 mL), and it was washed with saturated aqueous sodium sulfite solution (100 mL), saturated aqueous sodium carbonate solution (200 mL) in sequence. The organic fraction was washed with brine (150 mL), dried over Na₂SO₄, filtered through a Celite® pad and the filtrate was concentrated under reduced pressure to give a residue, which was purified by column chromatography on silica gel eluting with ethyl acetate (from 0% to 50%) in petroleum ether to give the title compound (11.5 g, 54.46 mmol, 97% yield) as a yellow solid.

LC-MS(ESI+): m/z 212.0 (M+H)⁺.

¹H NMR (400 MHz, CDCl₃) δ=8.35 (d, J=1.6 Hz, 1H), 8.30-8.20 (m, 1H), 7.70 (d, J=6.8 Hz, 1H), 3.96 (s, 3H), 3.93 (s, 3H).

### Dimethyl 2-chloropyridine-3,4-dicarboxylate

A solution of 3,4-bis(methoxycarbonyl)pyridine 1-oxide (10.00 g, 47.36 mmol) in POCl₃ (100 mL) was stirred at 110° C. for 6 hrs. After cooling to room temperature, the volatiles were removed in vacuo. The residue was diluted with DCM (30 mL), quenched with water (100 mL) slowly at 0° C. Then, saturated NaHCO₃ aqueous solution was added to adjust pH=7. The aqueous phase was extracted with DCM (80 mL×3). The combined organic layers were washed with brine (100 mL), dried over Na₂SO₄, filtered through a Celite® pad and the filtrate was concentrated under reduced pressure to give a residue, which was purified by column chromatography on silica gel eluting with ethyl acetate (from 0% to 5%) in petroleum ether to give the title compound (3.9 g, 16.98 mmol, 36% yield) as a yellow solid.

LC-MS(ESI+): m/z 230.0 (M+H)⁺.

¹H NMR (400 MHz, CDCl₃) δ=8.59 (d, J=4.8 Hz, 1H), 7.79 (d, J=5.2 Hz, 1H), 4.01 (s, 3H), 3.95 (s, 3H).

### (2-chloropyridine-3,4-diyl)dimethanol

To a solution of dimethyl 2-chloropyridine-3,4-dicarboxylate (6.4 g, 27.87 mmol) in EtOH (60 mL) was added NaBH₄ (6.33 g, 167.22 mmol) in portions at 0° C. The mixture was stirred at room temperature for 2 hrs. Formic acid was added to adjust pH=5, and the volatiles were removed under reduced pressure. The residue was purified by column chromatography on silica gel eluting with ethyl acetate (from 30% to 67%) in petroleum ether to give the title compound to give the title compound (2.3 g, 13.25 mmol, 48% yield) as a yellow solid.

LC-MS(ESI+): m/z 174.1 (M+H)⁺.

¹H NMR (400 MHz, DMSO-d₆) δ=8.31 (d, J=4.8 Hz, 1H), 7.53 (d, J=5.2 Hz, 1H), 5.63-5.45 (m, 1H), 5.25-5.10 (m, 1H), 4.75 (d, J=5.2 Hz, 2H), 4.59 (d, J=5.2 Hz, 2H).

### 2-chloro-3,4-bis(chloromethyl)pyridine

To a solution of (2-chloropyridine-3,4-diyl)dimethanol (2.30 g, 13.25 mmol) in DCM (20 mL) was added SOCl₂ (20 mL). The mixture was stirred at 35° C. for 2 hrs under N₂. The volatiles were removed in vacuo. Saturated aqueous sodium carbonate solution was added to the residue to adjust the pH=8. The resulting solution was extracted with DCM (50 mL×2). The combined organic layers were washed with brine (50 mL), dried over Na₂SO₄, filtered through a Celite® pad and the filtrate was concentrated under reduced pressure to give a residue, which was purified by column chromatography on silica gel eluting with ethyl acetate (from 0% to 10%) in petroleum ether to give the title compound (1.00 g, 4.75 mmol, 36% yield) as a yellow solid.

LC-MS(ESI+): m/z 210.0 (M+H)⁺.

¹H NMR (400 MHz, DMSO-d₆) δ=8.47 (d, J=4.8 Hz, 1H), 7.61 (d, J=4.8 Hz, 1H), 4.98 (s, 2H), 4.96 (s, 2H).

### 1-chloro-6-(4-(1-methyl-4-(trifluoromethyl)-1H-imidazol-2-yl)phenyl)-6,7-dihydro-5H-cyclopenta[c]pyridine-6-carbonitrile

To a solution of 2-(4-(1-methyl-4-(trifluoromethyl)-1H-imidazol-2-yl)phenyl)acetonitrile (700.0 mg, 2.64 mmol) and 2-chloro-3,4-bis(chloromethyl)pyridine (834.0 mg, 3.96 mmol) and in THF (10 mL) was added lithium bis(trimethylsilyl)amide (LiHMDS) (1 M in THF) (5.28 mL, 5.28 mmol) dropwise at 0° C. The mixture was stirred at room temperature for 2 hrs. To the reaction mixture was added water (40 mL) and the aqueous phase was extracted with EtOAc (20 mL×3). The combined organic layers were washed with brine (30 mL), dried over Na₂SO₄, filtered through a Celite® pad and the filtrate was concentrated under reduced pressure to give a residue, which was purified by column chromatography on silica gel eluting with ethyl acetate (from 0% to 30%) in petroleum ether to give the title compound (310 mg, 0.77 mmol, 29% yield) as a yellow solid.

LC-MS(ESI+): m/z 403.0 (M+H)⁺.

### 1-(2-isopropylpyridin-3-yl)-6-(4-(1-methyl-4-(trifluoromethyl)-1H-imidazol-2-yl)phenyl)-6,7-dihydro-5H-cyclopenta[c]pyridine-6-carbonitrile

To a solution of 1-chloro-6-(4-(1-methyl-4-(trifluoromethyl)-1H-imidazol-2-yl)phenyl)-6,7-dihydro-5H-cyclopenta[c]pyridine-6-carbonitrile (350.0 mg, 0.87 mmol) in dioxane (10 mL) and H₂O (2 mL) were added 2-isopropyl-3-(4,4,5,5-tetramethyl-1,3,2-dioxaborolan-2-yl)pyridine (2.15 g, 8.70 mmol), Pd(dppf)Cl₂ (66.0 mg, 0.09 mmol) and K₂CO₃ (240.0 mg, 1.74 mmol). The reaction was stirred at 100° C. for 16 hrs. To the cooled reaction mixture was added water (30 mL) and the aqueous phase was extracted with EtOAc (20 mL×2). The combined organic layers were washed with brine (30 mL), dried over Na₂SO₄, filtered through a Celite® pad and the filtrate was concentrated under reduced pressure to give a residue, which was purified by column chromatography on silica gel eluting with ethyl acetate (from 0% to 100%) in petroleum ether to give the title compound (120.0 mg, 0.25 mmol, 29% yield) as a brown solid.

LC-MS(ESI+): m/z 488.2 (M+H)⁺.

### 1-(2-isopropylpyridin-3-yl)-6-(4-(1-methyl-4-(trifluoromethyl)-1H-imidazol-2-yl)phenyl)-6,7-dihydro-5H-cyclopenta[c]pyridine-6-carboxylic Acid

To a solution of 1-(2-isopropylpyridin-3-yl)-6-(4-(1-methyl-4-(trifluoromethyl)-1H-imidazol-2-yl)phenyl)-6,7-dihydro-5H-cyclopenta[c]pyridine-6-carbonitrile (105.0 mg, 0.22 mmol) in EtOH (1 mL) and H₂O (1 mL) was added KOH (62.0 mg, 1.10 mmol). The reaction was stirred at 110° C. for 16 hrs. The volatiles were removed in vacuo. To the residue was added water (10 mL) and the aqueous phase was extracted with EtOAc (20 mL×2). The combined organic layers were washed with brine (15 mL), dried over Na₂SO₄, filtered through a Celite® pad and the filtrate was concentrated under reduced pressure to give a residue, which was purified by pre-TLC (DCM/MeOH=10/1) to give the title compound (34.0 mg, 0.07 mmol, 32% yield) as a brown solid.

LC-MS(ESI+): m/z 507.1 (M+H)⁺.

### 1-(2-isopropylpyridin-3-yl)-6-(4-(1-methyl-4-(trifluoromethyl)-1H-imidazol-2-yl)phenyl)-6,7-dihydro-5H-cyclopenta[c]pyridine

To a solution of 1-(2-isopropylpyridin-3-yl)-6-(4-(1-methyl-4-(trifluoromethyl)-1H-imidazol-2-yl)phenyl)-6,7-dihydro-5H-cyclopenta[c]pyridine-6-carboxylic acid (30.0 mg, 0.06 mmol) in DMSO (30 mL) and H₂O (10 mL) was added LiCl (12.7 mg, 0.30 mmol). The reaction was stirred at 140° C. for 24 hrs. To the cooled reaction mixture was added water (20 mL) and the aqueous phase was extracted with EtOAc (20 mL×2). The combined organic layers were washed with brine (10 mL), dried over Na₂SO₄, filtered through a Celite® pad and the filtrate was concentrated under reduced pressure to give a residue, which was purified by pre-TLC (DCM/EA=1/1) to give the title compound.

LC-MS(ESI+): m/z 463.1 (M+H)⁺.

¹H NMR (400 MHz, DMSO-d₆) δ=8.66-8.55 (m, 1H), 8.49 (d, J=5.2 Hz, 1H), 7.91 (s, 1H), 7.75-7.53 (m, 3H), 7.43 (d, J=8.0 Hz, 2H), 7.39 (d, J=4.8 Hz, 1H), 7.35-7.22 (m, 1H), 3.88-3.70 (m, 4H), 3.56-3.45 (m, 1H), 3.22-2.85 (m, 4H), 1.08 (d, J=6.4 Hz, 6H).

### Example B36: Synthesis of Intermediate 27-4

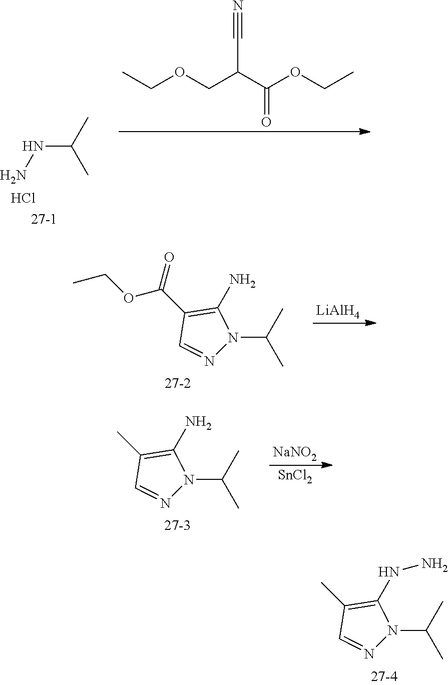

### Ethyl 5-amino-1-(prop-2-yl)pyrazole-4-carboxylate

A mixture of ethyl (2E)-2-cyano-3-ethoxyprop-2-enoate (7.65 g, 45.21 mmol), prop-2-yldiazene hydrochloride (5.00 g, 45.21 mmol) and K₂CO₃ (6.25 g, 45.21 mmol) in ethanol/methanol (50 mL/5 mL) was stirred at 80° C. for 18 hours. To the cooled reaction mixture was added water (50 mL) and the aqueous phase was extracted with EtOAc (50 mL×2). The combined organic layers were washed with brine (50 mL), dried over Na₂SO₄, filtered through a Celite® pad and the filtrate was concentrated under reduced pressure to give a residue, which was purified by column chromatography on silica gel eluting with ethyl acetate (from 0% to 60%) in petroleum ether to give the title compound (6.60 g, 33.47 mmol, 74% yield) as a yellow oil.

¹H NMR (400 MHz, CDCl₃) δ=7.64 (s, 1H), 5.04 (br.s, 2H), 4.35-4.24 (m, 2H), 4.21-4.08 (m, 1H), 1.46 (d, J=6.4 Hz, 6H), 1.33 (t, J=7.0 Hz, 3H).

### 4-methyl-2-(prop-2-yl)pyrazol-3-amine

To a solution of ethyl 5-amino-1-(prop-2-yl)pyrazole-4-carboxylate (6.40 g, 32.45 mmol) in dioxane (70 mL) was added lithium aluminium tetrahydride (4.93 g, 129.82 mmol) in portions at 0° C. The reaction mixture was stirred at 100° C. for 4 hrs. To the reaction mixture were added water (4.9 mL), 10% sodium hydroxide solution (4.9 mL) and water (14.7 mL) in sequence at 0° C. After stirring at room temperature for 0.5 hr, Na₂SO₄ was added into the mixture. The suspension was filtered through a Celite® pad and the filtrate was concentrated under reduced pressure to give a residue, which was purified by column chromatography on silica gel eluting with MeOH (from 0% to 30%) in DCM to give the title compound (4.00 g, 28.74 mmol, 89% yield) as a yellow oil.

LC-MS(ESI+): m/z 140.3 (M+H)⁺.

¹H NMR (400 MHz, CDCl₃) δ=7.20 (d, J=2.8 Hz, 1H), 4.45-4.33 (m, 1H), 3.21 (br.s, 2H), 1.93 (d, J=2.0 Hz, 3H) 1.55-1.40 (m, 6H).

### 5-hydrazinyl-1-isopropyl-4-methyl-1H-pyrazole

To a solution of 4-methyl-2-(prop-2-yl)pyrazol-3-amine (2.00 g, 14.37 mmol) in 6N HCl (25 mL) was added the solution of NaNO₂ (10.99 g, 14.37 mmol) in H₂O (15 mL) dropwise at −10° C. After stirring at −10° C. for 1 hr, a solution of SnCl₂ (5.45 g, 28.74 mmol) in 6N HCl (25 mL) was added dropwise. The reaction mixture was warmed to room temperature and stirred for another 1 hr. The mixture was filtered through a Celite® pad and the filtrate was purified by reversed phase column (H₂O/ACN from 100:0) to give the title compound (1.5 g, 9.73 mmol, 67.7% yield) as a white solid.

LC-MS(ESI+): m/z 155.3 (M+H)⁺.

### Example B37: Synthesis of Compound 27

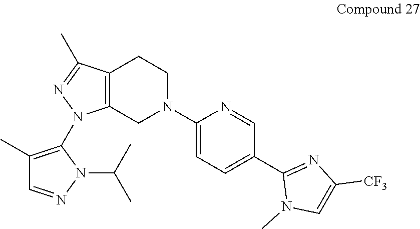 [SMILES: Cc1cnn(C(C)C)c1-n1nc(C)c2c1CN(c1ccc(-c3nc(C(F)(F)F)cn3C)cn1)CC2]

The synthetic route for Compound 27 was similar to that of Compound 13 by replacing intermediate 13-3 with 27-4, and replacing intermediate H with intermediate C, respectively. The crude product was purified by pre-TLC(PE/EA=1/1) to give the title compound.

LC-MS(ESI+): m/z 485.2 (M+H)⁺.

¹H NMR (400 MHz, DMSO-d₆) δ=8.38 (d, J=2.0 Hz, 1H), 7.95-7.80 (m, 2H), 7.49 (s, 1H), 7.12 (d, J=9.2 Hz, 1H), 4.76-4.45 (m, 2H), 4.02-3.96 (m, 2H), 3.95-3.83 (m, 1H), 3.73 (s, 3H), 2.72-2.55 (m, 2H), 2.16 (s, 3H), 1.87 (s, 3H), 1.30 (d, J=6.8 Hz, 6H).

### Example B38: Synthesis of Compound 28

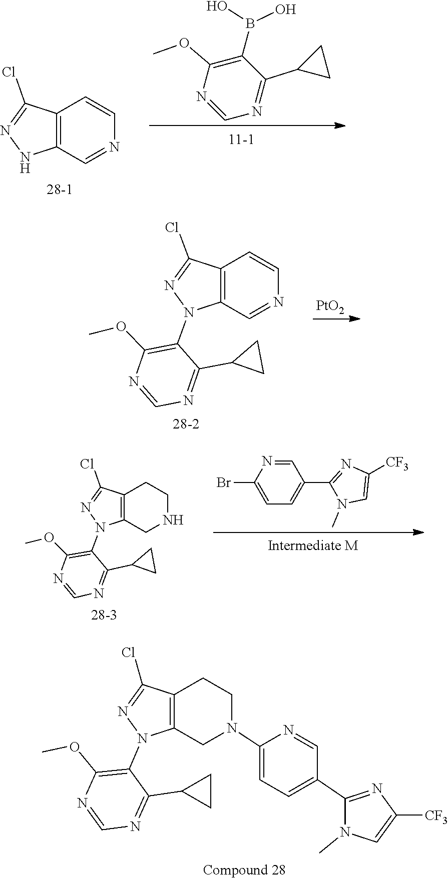

### 3-chloro-1-(4-cyclopropyl-6-methoxypyrimidin-5-yl)-1H-pyrazolo[3,4-c]pyridine

To a solution of 3-chloro-1H-pyrazolo[3,4-c]pyridine (500.0 mg, 3.26 mmol) and (4-cyclopropyl-6-methoxypyrimidin-5-yl)boronic acid (1260.9 mg, 6.50 mmol) in dioxane (10 mL) was added Copper(II) acetate monohydrate (194.0 mg, 0.97 mmol) and DBU (990.0 mg, 6.50 mmol). The mixture was stirred at room temperature for 16 hrs. To the reaction mixture was added water (50 mL) and the aqueous phase was extracted with EtOAc (50 mL×3). The combined organic layers were washed with brine (50 mL), dried over Na₂SO₄, filtered through a Celite® pad and the filtrate was concentrated under reduced pressure to give a residue, which was purified by column chromatography on silica gel eluting with ethyl acetate (from 0% to 25%) in petroleum ether to give the title compound (260.0 mg, 0.86 mmol, 26% yield) as a white solid.

LC-MS (ESI+): m/z 302.0 (M+H)⁺.

¹H NMR (400 MHz, DMSO-d₆) δ=8.97 (s, 1H), 8.81 (s, 1H), 8.49 (d, J=5.6 Hz, 1H), 7.95-7.80 (m, 1H), 3.90 (s, 3H), 1.63-1.50 (m, 1H), 1.23-0.75 (m, 4H).

### 3-chloro-1-(4-cyclopropyl-6-methoxypyrimidin-5-yl)-4,5,6,7-tetrahydro-1H-pyrazolo[3,4-c]pyridine

A mixture of 3-chloro-1-(4-cyclopropyl-6-methoxypyrimidin-5-yl)-1H-pyrazolo[3,4-c]pyridine (260.0 mg, 0.86 mmol) and Platinum(IV) oxide (117.0 mg, 0.51 mmol) in EtOH (6 mL) was stirred at room temperature for 16 hrs under H₂. The mixture was filtered through a Celite® pad and the filtrate was concentrated under reduced pressure to give a residue, which was purified by pre-TLC (DCM/MeOH=20/1) to give the title compound (200.0 mg, 0.65 mmol, 76% yield) as a white solid.

LC-MS (ESI+): m/z 306.0 (M+H)⁺.

¹H NMR (400 MHz, DMSO-d₆) δ=8.70 (s, 1H), 3.92 (s, 3H), 3.65-3.50 (m, 2H), 3.00-2.88 (m, 2H), 2.48-2.40 (m, 2H), 1.56-1.50 (m, 1H), 1.14-0.99 (m, 4H).

### 3-chloro-1-(4-cyclopropyl-6-methoxypyrimidin-5-yl)-6-(5-(1-methyl-4-(trifluoromethyl)-1H-imidazol-2-yl)pyridin-2-yl)-4,5,6,7-tetrahydro-1H-pyrazolo[3,4-c]pyridine

To a solution of 3-chloro-1-(4-cyclopropyl-6-methoxypyrimidin-5-yl)-4,5,6,7-tetrahydro-1H-pyrazolo[3,4-c]pyridine (70.0 mg, 0.23 mmol) in toluene (2 mL) were added 2-bromo-5-(1-methyl-4-(trifluoromethyl)-1H-imidazol-2-yl)pyridine (140.0 mg, 0.46 mmol), Pd(OAc)₂ (5 mg, 0.02 mmol), X-Phos (218.0 mg, 0.46 mmol) and t-BuONa (44.0 mg, 0.46 mmol). Then, the reaction was stirred at 100° C. for 16 hrs. To the cooled reaction mixture was added water (20 mL) and the aqueous phase was extracted with EtOAc (20 mL×3). The combined organic layers were washed with brine (30 mL×2), dried over Na₂SO₄, filtered through a Celite® pad and the filtrate was concentrated under reduced pressure to give a residue, which was purified by pre-TLC (DCM/EA=3/1) to give the title compound.

LC-MS(ESI+): m/z 531.1 (M+H)⁺.

¹H NMR (400 MHz, DMSO-d₆) δ=8.76 (s, 1H), 8.43 (d, J=2.4 Hz, 1H), 7.95-7.83 (m, 2H), 7.11 (d, J=8.8 Hz, 1H), 4.70-4.50 (m, 2H), 4.02-3.97 (m, 2H), 3.96 (s, 3H), 3.74 (s, 3H), 2.64 (s, 2H), 1.65-1.50 (m, 1H), 1.23-0.98 (m, 4H).

### Example B39: Synthesis of Compound 29

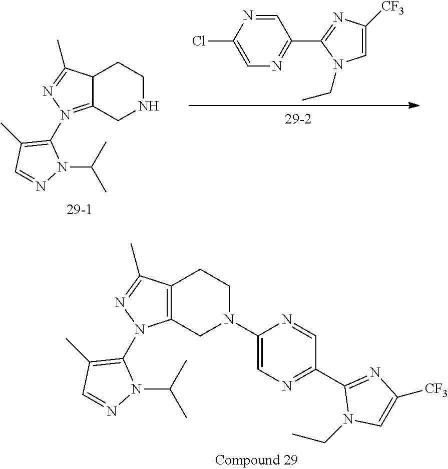

The synthetic route for intermediate 29-1 was similar to that of intermediate 11-3 by replacing intermediate 13-3 with 27-4.

The synthetic route for Intermediate 29-2 was similar to that of Intermediate C by replacing 6-fluoronicotinaldehyde with 5-chloropyrazine-2-carbaldehyde and replacing Mel with EtI, respectively.

### 6-(5-(1-ethyl-4-(trifluoromethyl)-1H-imidazol-2-yl)pyrazin-2-yl)-1-(1-isopropyl-4-methyl-1H-pyrazol-5-yl)-3-methyl-4,5,6,7-tetrahydro-1H-pyrazolo[3,4-c]pyridine

To a solution of 1-(1-isopropyl-4-methyl-1H-pyrazol-5-yl)-3-methyl-4,5,6,7-tetrahydro-1H-pyrazolo[3,4-c]pyridine (70.0 mg, 0.27 mmol), 2-chloro-5-(1-ethyl-4-(trifluoromethyl)-1H-imidazol-2-yl)pyrazine (112.0 mg, 0.40 mmol) in DMF (6 mL) was added TEA (81 mg, 0.81 mmol). The mixture was stirred at 100° C. for 16 hrs. To the cooled reaction mixture was added water (20 mL) and extracted with EtOAc (20 mL×3). The combined organic layers were washed with brine (30 mL×2), dried over Na₂SO₄, filtered through a Celite® pad and the filtrate was concentrated under reduced pressure to give a residue, which was purified by prep-TLC (PE/EA=3/1) to give the title compound.

LC-MS(ESI+): m/z 500.3 (M+H)⁺.

¹H NMR (400 MHz, DMSO-d₆) δ=8.66-8.60 (m, 1H), 8.56 (d, J=1.2 Hz, 1H), 8.00 (d, J=1.2 Hz, 1H), 7.49 (s, 1H), 4.60-4.50 (m, 2H), 4.44 (q, J=7.0 Hz, 2H), 4.16-4.00 (m, 1H), 3.99-3.90 (m, 2H), 2.72-2.60 (m, 2H), 2.17 (s, 3H), 1.87 (s, 3H), 1.36-1.29 (m, 9H).

### Example B40: Synthesis of Compound 30

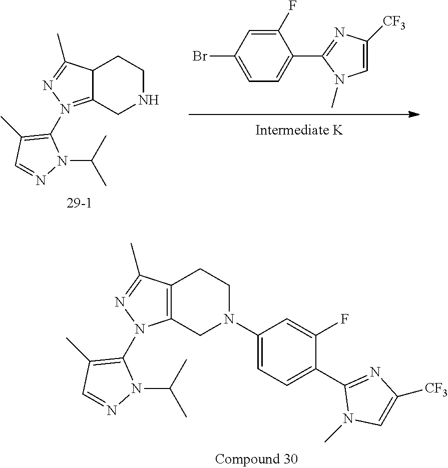

### 6-(3-fluoro-4-(1-methyl-4-(trifluoromethyl)-1H-imidazol-2-yl)phenyl)-1-(1-isopropyl-4-methyl-1H-pyrazol-5-yl)-3-methyl-4,5,6,7-tetrahydro-1H-pyrazolo[3,4-c]pyridine

To a solution of 1-(1-isopropyl-4-methyl-1H-pyrazol-5-yl)-3-methyl-4,5,6,7-tetrahydro-1H-pyrazolo[3,4-c]pyridine (40.0 mg, 0.15 mmol) and 2-(4-bromo-2-fluorophenyl)-1-methyl-4-(trifluoromethyl)-1H-imidazole (75.0 mg, 0.23 mmol) in toluene (3 mL) were added Pd(OAc)₂ (3.5 mg, 0.02 mmol), X-Phos (147.0 mg, 0.31 mmol) and sodium tert-butoxide (30.0 mg, 0.31 mmol). The mixture was stirred at 100° C. for 16 hrs. To the cooled reaction mixture was added water (30 mL) and extracted with EtOAc (20 mL×2). The combined organic layers were washed with brine (20 mL), dried over Na₂SO₄, filtered through a Celite® pad and the filtrate was concentrated under reduced pressure to give a residue, which was purified by pre-TLC (DCM/EtOAc=2/1) to give the title compound.

LC-MS(ESI+): m/z 502.2 (M+H)⁺.

¹H NMR (400 MHz, DMSO-d₆) δ=7.92 (s, 1H), 7.49 (s, 1H), 7.40-7.26 (m, 1H), 6.99-6.95 (m, 1H), 6.91-6.85 (m, 1H), 4.26-4.19 (m, 2H), 3.99-3.93 (m, 1H), 3.82-3.65 (m, 2H), 3.55 (s, 3H), 2.67-2.61 (m, 2H), 2.17 (s, 3H), 1.85 (s, 3H), 1.31-1.23 (m, 6H).

### Example B41: Synthesis of Intermediate 31-2

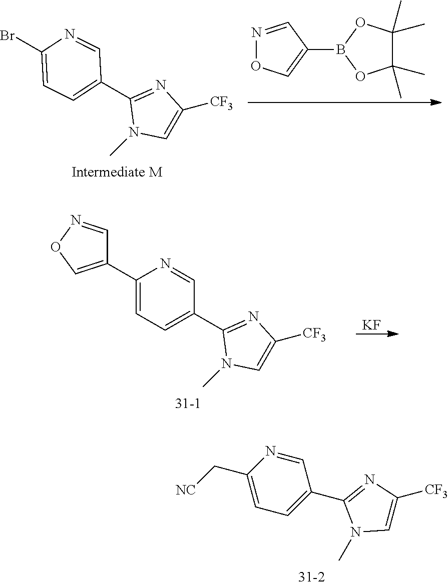

### 4-(5-(1-methyl-4-(trifluoromethyl)-1H-imidazol-2-yl)pyridin-2-yl)isoxazole

To a solution of 2-bromo-5-(1-methyl-4-(trifluoromethyl)-1H-imidazol-2-yl)pyridine (3.21 g, 10.49 mmol) and 4-(4,4,5,5-tetramethyl-1,3,2-dioxaborolan-2-yl)isoxazole (3.00 g, 15.38 mmol) in DMSO (30 mL) and H₂O (8 mL) were added Pd(dppf)Cl₂ (768.0 mg, 1.05 mmol) and potassium fluoride (1.20 g, 20.65 mmol). The mixture was stirred at 110° C. for 16 hrs under N₂. To the cooled reaction mixture was added water (50 mL) and extracted with EtOAc (50 mL×2). The combined organic layers were washed with brine (50 mL), dried over Na₂SO₄, filtered through a Celite® pad and the filtrate was concentrated under reduced pressure to give a residue, which was purified by column chromatography on silica gel eluting with ethyl acetate (from 0% to 100%) in petroleum ether to give the title compound (1.60 g, 5.44 mmol, 52% yield) as a brown solid.

LC-MS(ESI−): m/z 293.1 (M−H)⁻.

### 2-(5-(1-methyl-4-(trifluoromethyl)-1H-imidazol-2-yl)pyridin-2-yl)acetonitrile

To a solution of 4-(5-(1-methyl-4-(trifluoromethyl)-1H-imidazol-2-yl)pyridin-2-yl)isoxazole (1.60 g, 5.44 mmol) in MeOH (12 mL) and H₂O (4 mL) was added potassium fluoride (790.0 mg, 13.60 mmol). The mixture was stirred at 90° C. for 6 hrs. To the cooled reaction mixture was added water (20 mL) and extracted with DCM (30 mL×2). The combined organic layers were washed with brine (15 mL), dried over Na₂SO₄, filtered through a Celite® pad and the filtrate was concentrated under reduced pressure to give a residue, which was purified by column chromatography on silica gel eluting with ethyl acetate (from 0% to 30%) in petroleum ether to give the title compound (1.06 g, 3.98 mmol, 73% yield) as a brown solid.

¹H NMR (400 MHz, DMSO-d₆) δ=8.92-8.05 (m, 1H), 8.21-8.18 (m, 1H), 8.02 (s, 1H), 7.57 (d, J=8.4 Hz, 1H), 4.33 (s, 2H), 3.83 (s, 3H).

### Example B42: Synthesis of Compound 31

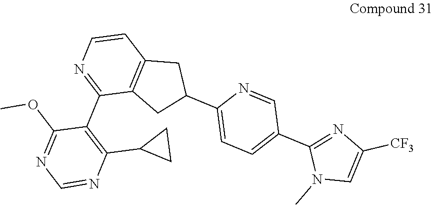 [SMILES: COc1ncnc(C2CC2)c1-c1nccc2c1CC(c1ccc(-c3nc(C(F)(F)F)cn3C)cn1)C2]

The synthetic route for Compound 31 was similar to that of Compound 26 by replacing intermediate 26-1 with intermediate 31-2 and replacing intermediate A with intermediate D, respectively. The crude product was purified by prep-TLC (DCM/EA=3/1) to give the title compound.

LC-MS(ESI+): m/z 493.2 (M+H)⁺.

¹H NMR (400 MHz, DMSO-d₆) δ=8.86-8.78 (m, 1H), 8.68-8.60 (m, 1H), 8.54-8.46 (m, 1H), 8.15-8.05 (m, 1H), 8.02-7.95 (m, 1H), 7.54-7.46 (m, 1H), 7.39-7.34 (m, 1H), 4.00-3.94 (m, 1H), 3.87-3.78 (m, 6H), 3.54-3.36 (m, 2H), 3.15-2.96 (m, 2H), 1.70-1.51 (m, 1H), 1.11-0.87 (m, 4H).

### Example B43: Synthesis of Compound 32

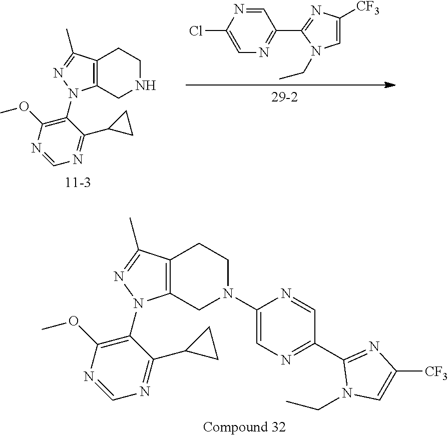 [SMILES: COc1ncnc(C2CC2)c1-n1nc(C)c2c1CNCC2 ; CCn1cc(C(F)(F)F)nc1-c1cnc(Cl)cn1 ; CCn1cc(C(F)(F)F)nc1-c1cnc(N2CCc3c(C)nn(-c4c(OC)ncnc4C4CC4)c3C2)cn1]

### 1-(4-cyclopropyl-6-methoxypyrimidin-5-yl)-6-(5-(1-ethyl-4-(trifluoromethyl)-1H-imidazol-2-yl)pyrazin-2-yl)-3-methyl-4,5,6,7-tetrahydro-1H-pyrazolo[3,4-c]pyridine

To a solution of 1-(4-cyclopropyl-6-methoxypyrimidin-5-yl)-3-methyl-4,5,6,7-tetrahydro-1H-pyrazolo[3,4-c]pyridine (70.0 mg, 0.25 mmol), 2-chloro-5-(1-ethyl-4-(trifluoromethyl)-1H-imidazol-2-yl)pyrazine (145.0 mg, 0.52 mmol) in DMF (6 mL) was added TEA (106.4 mg, 1.051 mmol). The mixture was stirred at 100° C. for 16 hrs. To the cooled reaction mixture was added water (20 mL) and extracted with EtOAc (20 mL×2). The combined organic layers were washed with brine (20 mL×2), dried over Na₂SO₄, filtered through a Celite® pad and the filtrate was concentrated under reduced pressure to give a residue, which was purified by pre-TLC (DCM/EA=2/1) to give the title compound.

LC-MS(ESI+): m/z 526.1 (M+H)⁺.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having the structure of Formula (I), or a pharmaceutically acceptable salt thereof, 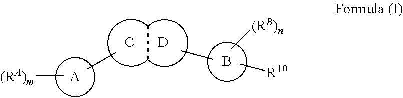
wherein,
ring A is aryl or heteroaryl;
ring B is phenyl, phenyl isostere, or heteroaryl;
ring C is an optionally substituted 5 to 6 membered heteroaryl;
ring D is an optionally substituted 5 to 6 membered heterocycloalkyl or an optionally substituted 5 to 6 membered cycloalkyl;
each of R^{A} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{A1};
or two R^{A} are taken together with the intervening atoms to which they are attached to form a cycloalkyl, heterocycloalkyl, aryl, or heteroaryl, wherein each of which independently optionally substituted with one or more R^{A1};
each R^{A1} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl;
wherein each of the alkyl, heteroalkyl, alkenyl, alkynyl, cycloalkyl, heterocycloalkyl, aryl, and heteroaryl is independently optionally substituted with one or more R;
m is 0, 1, 2, 3, 4, or 5;
each of R^{B} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl, wherein each of the alkyl, heteroalkyl, alkenyl, alkynyl, cycloalkyl, heterocycloalkyl, aryl, and heteroaryl is independently optionally substituted with one or more R^{B1};
n is 0, 1, 2, 3, or 4;
each R^{B1} is independently selected from halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, or heteroaryl;
wherein each of the alkyl, heteroalkyl, alkenyl, alkynyl, cycloalkyl, heterocycloalkyl, aryl, and heteroaryl is independently optionally substituted with one or more R;
R¹⁰ is cycloalkyl, heterocycloalkyl, aryl, or heteroaryl, each of which is independently optionally substituted with one or more R^{B1};
each R^{a} is independently C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
each R^{b} is independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
R^{c} and R^{d} are each independently hydrogen, C₁-C₆ alkyl, C₁-C₆ haloalkyl, C₁-C₆ hydroxyalkyl, C₁-C₆ aminoalkyl, C₁-C₆ heteroalkyl, C₂-C₆ alkenyl, C₂-C₆ alkynyl, cycloalkyl, heterocycloalkyl, aryl, heteroaryl, C₁-C₆ alkylene(cycloalkyl), C₁-C₆ alkylene(heterocycloalkyl), C₁-C₆ alkylene(aryl), or C₁-C₆ alkylene(heteroaryl), wherein each alkyl, alkylene, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
each R is independently halogen, —CN, —OH, —SF₅, —SH, —S(═O)C₁-C₃ alkyl, —S(═O)₂C₁-C₃ alkyl, —S(═O)₂NH₂, —S(═O)₂NHC₁—C₃ alkyl, —S(═O)₂N(C₁-C₃ alkyl)₂, —S(═O)(═NC₁—C₃ alkyl)(C₁-C₃ alkyl), —NH₂, —NHC₁—C₃ alkyl, —N(C₁-C₃ alkyl)₂, —N═S(═O)(C₁-C₃ alkyl)₂, —C(═O)C₁-C₃ alkyl, —C(═O)OH, —C(═O)OC₁—C₃ alkyl, —C(═O)NH₂, —C(═O)NHC₁—C₃ alkyl, —C(═O)N(C₁-C₃ alkyl)₂, —P(═O)(C₁-C₃ alkyl)₂, C₁-C₃ alkyl, C₁-C₃alkoxy, C₁-C₃ haloalkyl, C₁-C₃ haloalkoxy, C₁-C₃ hydroxyalkyl, C₁-C₃ aminoalkyl, C₁-C₃ heteroalkyl, or C₃-C₆cycloalkyl;
or two R on the same atom form an oxo.

2. The compound of claim 1, or a pharmaceutically acceptable salt thereof, wherein the ring A is phenyl or 5-6 membered monocyclic heteroaryl.

3. The compound of claim 1 or 2, or a pharmaceutically acceptable salt thereof, wherein the ring A is phenyl or 5-6 membered monocyclic heteroaryl.

4. The compound of any one of claim 1-3, or a pharmaceutically acceptable salt thereof, wherein 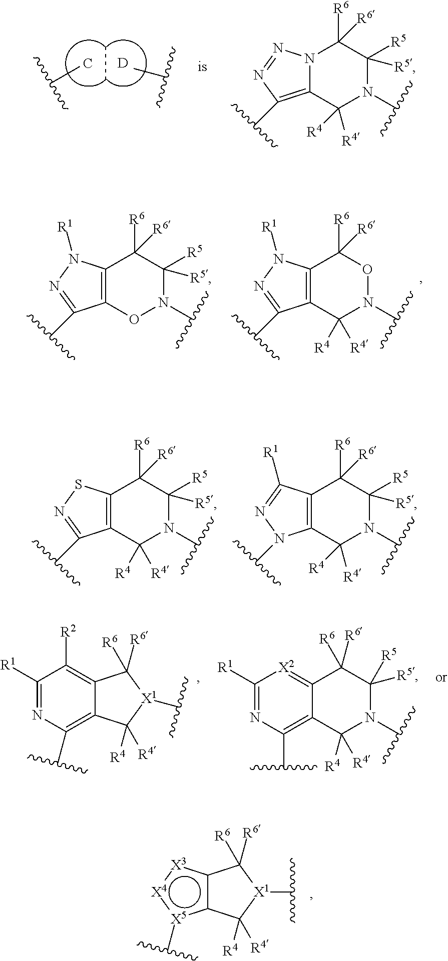
wherein
X¹ is N or CR^{X};
R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R;
X² is N or CR²;
X³ is N, NR^{1N}, CR¹, S or O;
X⁴ is N, NR^{2N}, CR², S or O;
X⁵ is N or C;
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

5. The compound of any one of claim 1 to 4, or a pharmaceutically acceptable salt thereof, wherein

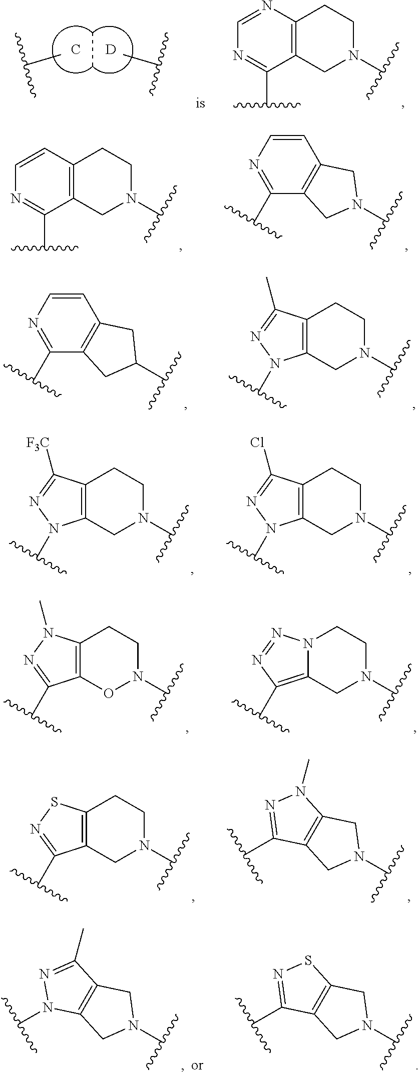

6. The compound of any one of claim 1 to 4, or a pharmaceutically acceptable salt thereof, wherein the compound has a structure of Formula (IIA)

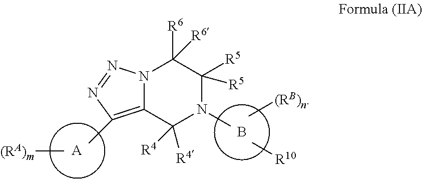

7. The compound of claim 1 or 2, or a pharmaceutically acceptable salt thereof, wherein the compound has a structure of Formula (IIB)

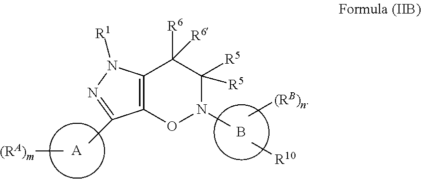

8. The compound of any one of claim 1 to 4, or a pharmaceutically acceptable salt thereof, wherein the compound has a structure of Formula (IIC)

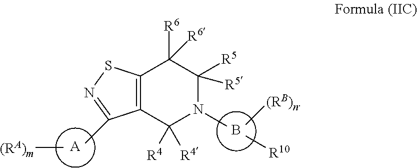

9. The compound of any one of claim 1 to 4, or a pharmaceutically acceptable salt thereof, wherein the compound has a structure of Formula (IID)

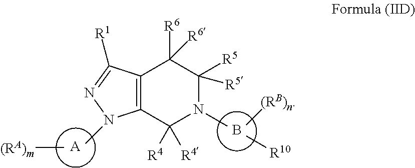

10. The compound of any one of claim 1 to 4, or 7, or a pharmaceutically acceptable salt thereof, wherein the compound has a structure of Formula (IID′) 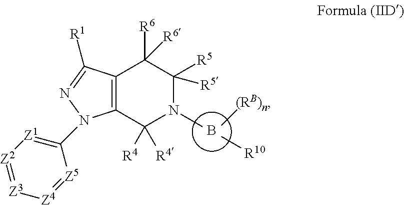
wherein,
Z¹ is N or CR^{Z1};
Z² is N or CR^{Z2};
Z³ is N or CR^{Z3};
Z⁴ is N or CR⁴;
Z⁵ is N or CR^{Z4};
each of R^{Z1}, R^{Z2}, R^{Z3}, R^{Z4} and R^{Z5} is independently hydrogen or R^{A}, provided that when Z¹ is CR^{Z1} and Z⁵ is CR^{Z5}, at least one of R^{Z1} and R^{Z5} is selected from R^{A};
R¹ is halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

11. The compound of any one of claim 1 to 4, or 7, or a pharmaceutically acceptable salt thereof, wherein the compound has a structure of Formula (IID″) 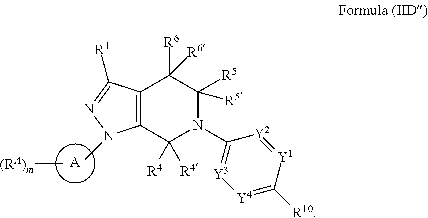
ring A is 5-membered heteroaryl;
Y¹ is N or CR^{Y1};
Y² is N or CR^{Y2};
Y³ is N or CR^{Y3};
Y⁴ is N or R⁴;
each of R^{Y1}, R^{Y2}, R^{Y3} and R^{Y4} is independently hydrogen or R^{B};
R¹ is hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

12. The compound of any one of claim 1 to 4, or a pharmaceutically acceptable salt thereof, wherein the compound has a structure of Formula (IIE) 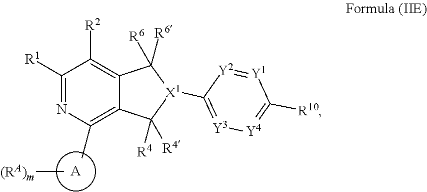
Y¹ is N or CR^{Y1};
Y² is N or CR^{Y2};
Y³ is N or CR^{Y3};
Y⁴ is N or CR^{Y4};
each of R^{Y1}, R^{Y2}, R^{Y3} and R^{Y4} is independently hydrogen or R^{B};
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
X¹ is N or CR^{X};
R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

13. The compound of claim 1 or 2, or a pharmaceutically acceptable salt thereof, wherein the compound has a structure of Formula (IIF) 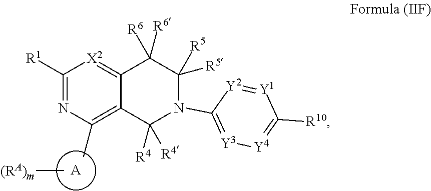
Y¹ is N or CR^{Y1};
Y² is N or CR^{Y2};
Y³ is N or CR^{Y3};
Y⁴ is N or R^{Y4};
X² is N or CR²;
each of R^{Y1}, R^{Y2}, R^{Y3} and R^{Y4} is independently hydrogen or R^{B};
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R;
each of R⁵ and R^{5′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁵ and R^{5′} are taken together to form an oxo; or R⁵ and R^{5′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

14. The compound of any one of claim 2 to 4, 12, or 11, or a pharmaceutically acceptable salt thereof, wherein R² is hydrogen, halogen, C₁₋₆ alkyl, or C₁-C₆ haloalkyl, wherein the alkyl is optionally substituted with one or more R.

15. The compound of any one of claim 2 to 4, 12, or 11, or a pharmaceutically acceptable salt thereof, wherein R² is hydrogen.

16. The compound of any one of claim 2 to 4, 6 to 11, or 13 to 15, or a pharmaceutically acceptable salt thereof, wherein R⁵ is selected from hydrogen, halogen, C₁₋₆ alkyl, and C₁-C₆ haloalkyl, wherein the alkyl is optionally substituted with one or more R.

17. The compound of any one of claim 2 to 4, 6 to 11, or 13 to 16, or a pharmaceutically acceptable salt thereof, wherein R^{5′} is selected from hydrogen, halogen, C₁₋₆ alkyl, and C₁-C₆ haloalkyl, wherein the alkyl is optionally substituted with one or more R.

18. The compound of any one of claim 1 to 4, or a pharmaceutically acceptable salt thereof, wherein the compound has a structure of Formula (IIG) 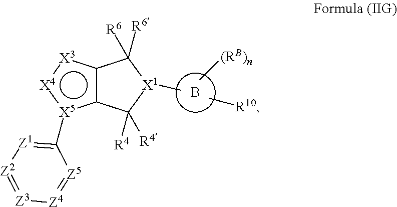
Z¹ is N or CR^{Z1};
Z² is N or CR^{Z2};
Z³ is N or CR^{Z3};
Z⁴ is N or CR^{Z4};
Z⁵ is N or CR^{Z4};
each of R^{Z1}, R^{Z2}, R^{Z3}, R^{Z4} and R^{Z5} is independently hydrogen or R^{A}, provided that at least one of R^{Z1} and R^{Z5} is present and is selected from R^{A};
X³ is N, NR^{1N}, CR¹, S or O;
X⁴ is N, NR^{2N}, CR², S or O;
X⁵ is N or C;
each of R¹ and R² is independently hydrogen, halogen, —CN, OH, —SF₅, —SH, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, or heterocycloalkyl, wherein each of the alkyl, heteroalkyl, cycloalkyl, or heterocycloalkyl is optionally substituted with one or more R;
each of R^{1N} and R^{2N} is independently hydrogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R;
X¹ is N or CR^{X};
R^{X} is hydrogen, halogen, C₁₋₆ alkyl, C₁-C₆ haloalkyl, C₁-C₆ hydroxyalkyl, C₁-C₆ aminoalkyl, or C₁-C₆ heteroalkyl, wherein each of the alkyl or heteroalkyl is optionally substituted with one or more R;
each of R⁴ and R^{4′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂-6 alkenyl, C₂-6 alkynyl, cycloalkyl, and heterocycloalkyl, wherein each of the alkyl, heteroalkyl, cycloalkyl, or heterocycloalkyl is optionally substituted with one or more R; or R⁴ and R^{4′} are taken together to form an oxo; or R⁴ and R^{4′} are taken together with the carbon to which they are attached to form a 3-6 membered cycloalkyl or 3-6 membered heterocycloalkyl, each of which is optionally substituted with one or more R; and
each of R⁶ and R^{6′} is independently selected from hydrogen, halogen, —CN, —OR^{a}, —SR^{a}, —NR^{c}R^{d}, C₁₋₆ alkyl, C₁-C₆ haloalkyl, C₁-C₆ hydroxyalkyl, C₁-C₆ aminoalkyl, C₁-C₆ heteroalkyl, C₂₋₆ alkenyl, C₂₋₆ alkynyl, cycloalkyl, and heterocycloalkyl, wherein each of the alkyl, heteroalkyl, cycloalkyl, or heterocycloalkyl is optionally substituted with one or more R; or R⁶ and R^{6′} are taken together to form an oxo; or R⁶ and R^{6′} are taken together with the carbon to which they are attached to form a 3-6 membered cycloalkyl or 3-6 membered heterocycloalkyl, each of which is optionally substituted with one or more R.

19. The compound of claim 18, or a pharmaceutically acceptable salt thereof, wherein the

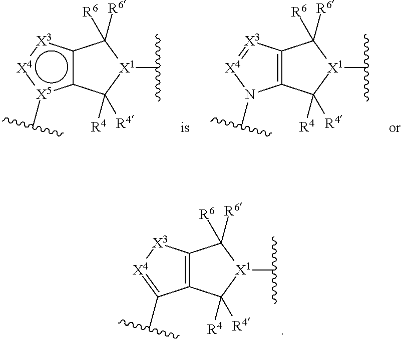

20. The compound of claim 18 or 19, or a pharmaceutically acceptable salt thereof, wherein
X³ is N, NR^{1N}, or CR¹.

21. The compound of any one of claim 18 to 20, or a pharmaceutically acceptable salt thereof, wherein R^{1N} is hydrogen, C₁₋₆ alkyl, or C₁-C₆ haloalkyl, wherein the alkyl is optionally substituted with one or more R.

22. The compound of any one of claim 1 to 4, 7, or 9 to 20, or a pharmaceutically acceptable salt thereof, wherein R¹ is halogen, C₁₋₆ alkyl, or C₁-C₆ haloalkyl, wherein the alkyl is optionally substituted with one or more R.

23. The compound of any one of claim 18 to 22, or a pharmaceutically acceptable salt thereof, wherein X⁴ is N, NR^{2N}, or CR².

24. The compound of any one of claim 10 or 18 to 23, or a pharmaceutically acceptable salt thereof, wherein each of R^{Z1}, R^{Z2}, R^{Z3} and R^{Z4} is hydrogen.

25. The compound of any one of claim 10, or 18 to 24, or a pharmaceutically acceptable salt thereof, wherein Z¹ is N.

26. The compound of any one of claim 10, or 18 to 24, or a pharmaceutically acceptable salt thereof, wherein Z¹ is CR^{Z1}.

27. The compound of claim 26, or a pharmaceutically acceptable salt thereof, wherein R^{Z1} is R^{A}, wherein R^{A} is selected from halogen, —OR^{a}, C₁-C₆ alkyl, C₁-C₆ haloalkyl, and cycloalkyl, wherein the alkyl and cycloalkyl is optionally substituted with one or more R^{A1}.

28. The compound of any one of claim 10 or 18 to 24, or a pharmaceutically acceptable salt thereof, wherein Z² is N.

29. The compound of any one of claim 10 or 18 to 24, or a pharmaceutically acceptable salt thereof, wherein Z² is CR^{Z2}.

30. The compound of claim 29, or a pharmaceutically acceptable salt thereof, wherein R^{Z2} is R^{A}, wherein R^{A} is selected from halogen, —OR^{a}, C₁-C₆ alkyl, C₁-C₆ haloalkyl, and cycloalkyl, wherein the alkyl and cycloalkyl is optionally substituted with one or more R^{A1}.

31. The compound of any one of claim 10 or 18 to 24, or a pharmaceutically acceptable salt thereof, wherein Z³ is N.

32. The compound of any one of claim 10 or 18 to 24, or a pharmaceutically acceptable salt thereof, wherein Z³ is CR^{Z3}.

33. The compound of claim 32, or a pharmaceutically acceptable salt thereof, wherein R^{Z3} is R^{A}, wherein R^{A} is selected from halogen, —OR^{a}, C₁-C₆ alkyl, C₁-C₆ haloalkyl, and cycloalkyl, wherein the alkyl and cycloalkyl is optionally substituted with one or more R^{A1}.

34. The compound of any one of claim 10 or 18 to 24, or a pharmaceutically acceptable salt thereof, wherein Z⁴ is N.

35. The compound of any one of claim 10 or 18 to 24, or a pharmaceutically acceptable salt thereof, wherein Z⁴ is CR^{Z4}.

36. The compound of claim 35, or a pharmaceutically acceptable salt thereof, wherein R^{Z4} is R^{A}, wherein R^{A} is selected from halogen, —OR^{a}, C₁-C₆ alkyl, C₁-C₆ haloalkyl, and cycloalkyl, wherein the alkyl and cycloalkyl is optionally substituted with one or more R^{A1}.

37. The compound of any one of claim 10 or 18 to 24, or a pharmaceutically acceptable salt thereof, wherein Z⁵ is N.

38. The compound of any one of claim 10 or 18 to 24, or a pharmaceutically acceptable salt thereof, wherein Z⁵ is CR^{Z5}.

39. The compound of claim 38, or a pharmaceutically acceptable salt thereof, wherein R^{Z5} is R^{A}, wherein R^{A} is selected from halogen, —OR^{a}, C₁-C₆ alkyl, C₁-C₆ haloalkyl, and cycloalkyl, wherein the alkyl and cycloalkyl is optionally substituted with one or more R^{A1}.

40. The compound of any one of claim 12 and 18 to 39, or a pharmaceutically acceptable salt thereof, wherein X¹ is CR^{X}.

41. The compound of claim 40, or a pharmaceutically acceptable salt thereof, wherein R^{X} is hydrogen, halogen, C₁₋₆ alkyl, or C₁-C₆ haloalkyl, wherein the alkyl is optionally substituted with one or more R.

42. The compound of any one of claim 1 to 4, 6, or 8 to 40, or a pharmaceutically acceptable salt thereof, wherein R⁴ is selected from hydrogen, halogen, C₁₋₆ alkyl, and C₁-C₆ haloalkyl, wherein the alkyl is optionally substituted with one or more R.

43. The compound of any one of claim 1 to 4, 6, or 8 to 42, or a pharmaceutically acceptable salt thereof, wherein R^{4′} is selected from hydrogen, halogen, C₁₋₆ alkyl, and C₁-C₆ haloalkyl, wherein the alkyl is optionally substituted with one or more R.

44. The compound of any one of claim 1 to 4, or 6 to 43, or a pharmaceutically acceptable salt thereof, wherein R⁶ is selected from hydrogen, halogen, C₁₋₆ alkyl, and C₁-C₆ haloalkyl, wherein the alkyl is optionally substituted with one or more R.

45. The compound of any one of claim 1 to 4, or 6 to 44, or a pharmaceutically acceptable salt thereof, wherein R^{6′} is hydrogen, halogen, C₁₋₆ alkyl, and C₁-C₆ haloalkyl, wherein the alkyl is optionally substituted with one or more R.

46. The compound of any one of claim 11 to 17, or 22 to 45, or a pharmaceutically acceptable salt thereof, wherein each of R^{Y1}, R^{Y2}, R^{Y3} and R^{Y4} is hydrogen.

47. The compound of any one of claim 11 to 17, or 22 to 45, or a pharmaceutically acceptable salt thereof, wherein Y¹ is N.

48. The compound of any one of claim 11 to 17, or 22 to 46, or a pharmaceutically acceptable salt thereof, wherein Y¹ is CR^{Y1}.

49. The compound of claim 48, or a pharmaceutically acceptable salt thereof, wherein R^{Y1} is R^{B}, wherein R^{B} is selected from halogen, C₁-C₆ alkyl, and C₁-C₆ haloalkyl, wherein the alkyl is optionally substituted with one or more R^{B1}.

50. The compound of any one of claim 11 to 17, 22 to 45, or 47 to 49, or a pharmaceutically acceptable salt thereof, wherein Y² is N.

51. The compound of any one of claim 11 to 17, 22 to 45, or 47 to 49, or a pharmaceutically acceptable salt thereof, wherein Y² is CR^{Y2}.

52. The compound of claim 51, or a pharmaceutically acceptable salt thereof, wherein R^{Y2} is R^{B}, wherein R^{B} is selected from halogen, C₁-C₆ alkyl, and C₁-C₆ haloalkyl, wherein the alkyl is optionally substituted with one or more R^{B1}.

53. The compound of any one of claim 11 to 17, 22 to 45, or 47 to 52, or a pharmaceutically acceptable salt thereof, wherein Y³ is N.

54. The compound of any one of claim 11 to 17, 22 to 45, or 47 to 52, or a pharmaceutically acceptable salt thereof, wherein Y³ is CR³.

55. The compound of claim 54, or a pharmaceutically acceptable salt thereof, wherein R^{Y3} is R^{B}, wherein R^{B} is selected from halogen, C₁-C₆ alkyl, and C₁-C₆ haloalkyl, wherein the alkyl is optionally substituted with one or more R^{B1}.

56. The compound of any one of claim 11 to 17, 22 to 45, or 47 to 55, or a pharmaceutically acceptable salt thereof, wherein Y⁴ is N.

57. The compound of any one of claim 11 to 17, 22 to 45, or 47 to 55, or a pharmaceutically acceptable salt thereof, wherein Y⁴ is CR^{Y4}.

58. The compound of claim 57, or a pharmaceutically acceptable salt thereof, wherein R^{Y4} is R^{B}, wherein R^{B} is selected from halogen, C₁-C₆ alkyl, and C₁-C₆ haloalkyl, wherein the alkyl is optionally substituted with one or more R^{B1}.

59. The compound of any one of claim 1 to 9, 11 to 17, 22 to 24, or 40 to 58, or a pharmaceutically acceptable salt thereof, wherein ring A is phenyl.

60. The compound of any one of claim 1 to 9, 11 to 17, 22 to 24, or 40 to 58, or a pharmaceutically acceptable salt thereof, wherein ring A is 5-6 membered monocyclic heteroaryl.

61. The compound of claim 60, or a pharmaceutically acceptable salt thereof, wherein ring A is pyridine, pyrimidine, pyrazine, pyridazine, triazine, imidazole, pyrazole, triazole, oxazole, isoxazole, thiophene, or thiazole.

62. The compound of claim 60, or a pharmaceutically acceptable salt thereof, wherein

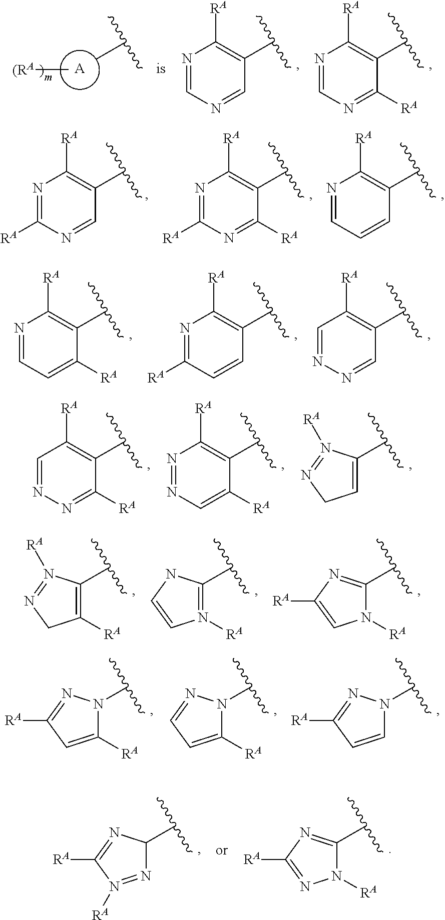

63. The compound of claim 60, or a pharmaceutically acceptable salt thereof, wherein 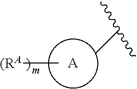
is selected from:

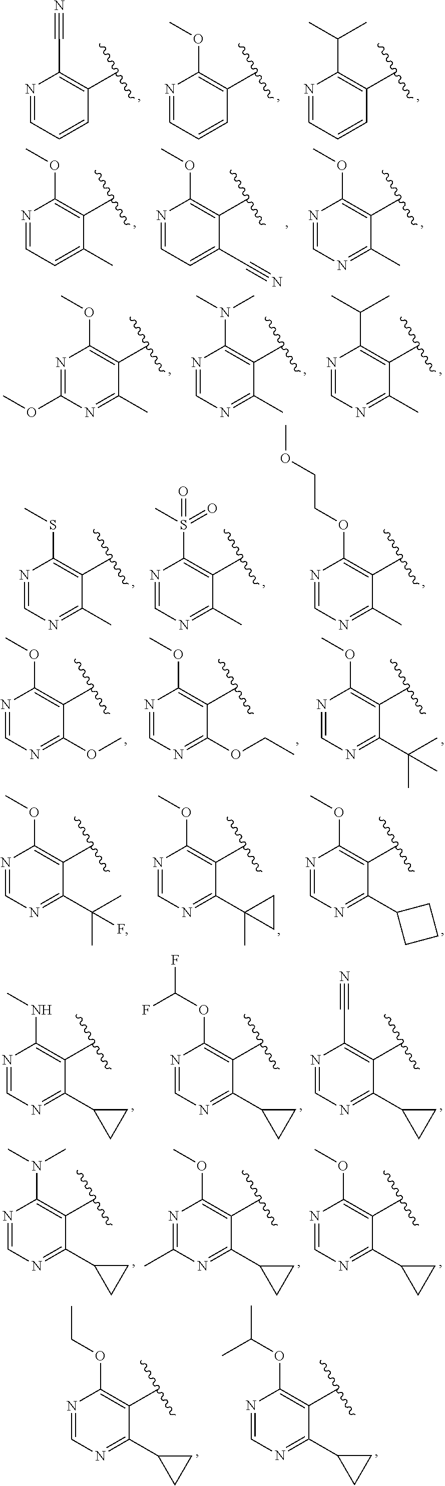

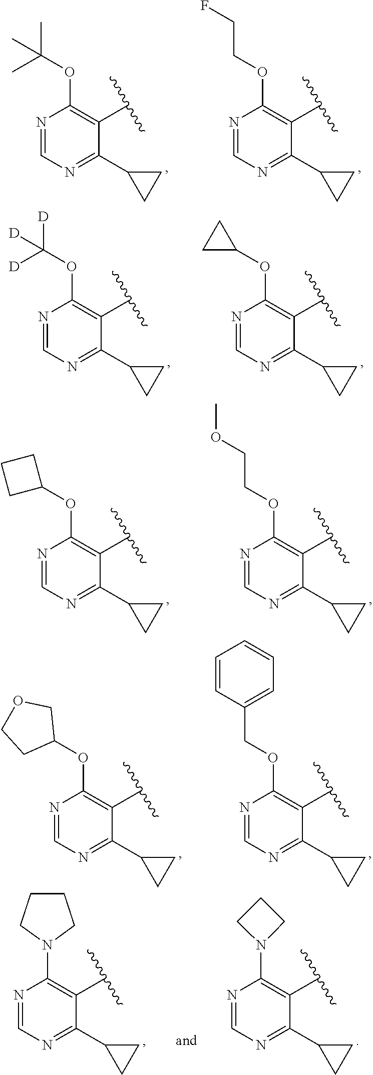

64. The compound of claim 60, or a pharmaceutically acceptable salt thereof, wherein 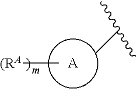
is selected from

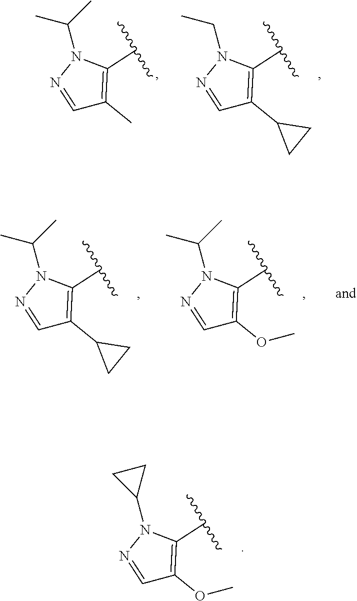

65. The compound of claim 60, or a pharmaceutically acceptable salt thereof, wherein 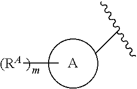
is selected from:

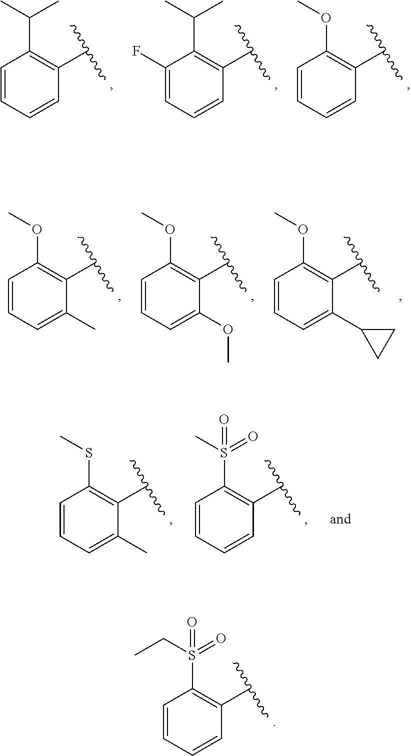

66. The compound of any one of claim 1 to 9 or 14 to 65, or a pharmaceutically acceptable salt thereof, wherein ring B is phenyl.

67. The compound of any one of claim 1 to 9 or 14 to 65, or a pharmaceutically acceptable salt thereof, wherein ring B is phenyl isostere.

68. The compound of claim 67, pharmaceutically acceptable salt thereof, wherein the phenyl isostere is cubane.

69. The compound of any one of claim 1 to 9 or 14 to 65, or a pharmaceutically acceptable salt thereof, wherein ring B is 5-6 membered monocyclic heteroaryl.

70. The compound of claim 69, or a pharmaceutically acceptable salt thereof, wherein ring B is pyridine, pyrimidine, pyrazine, pyridazine, triazine, imidazole, pyrazole, triazole, oxazole, isoxazole, thiophene, or thiazole.

71. The compound of claim 69, or a pharmaceutically acceptable salt thereof, wherein 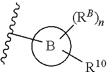
is selected from:

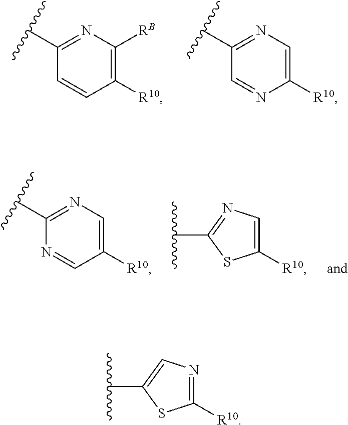

72. The compound of any one of claim 1 to 71, or a pharmaceutically acceptable salt thereof, wherein R¹⁰ is heteroaryl, wherein the heteroaryl is optionally substituted with one or more R^{B1}.

73. The compound of claim 72, or a pharmaceutically acceptable salt thereof, wherein R¹⁰ is an optionally substituted 5 membered monocyclic heteroaryl with 1 to 4 heteroatoms selected from N, O, S, and P.

74. The compound of claim 73, or a pharmaceutically acceptable salt thereof, wherein R¹⁰ is imidazole, pyrazole, triazole, or tetrazole, each of which optionally substituted.

75. The compound of claim 72, or a pharmaceutically acceptable salt thereof, wherein R¹⁰ is optionally substituted fused 5-6, 6-6 or 6-5 heteroaryl.

76. The compound of any one of claim 1 to 75, or a pharmaceutically acceptable salt thereof, wherein R^{B1} is halogen, C₁-C₆ alkyl, or C₁-C₆ haloalkyl; wherein alkyl is optionally substituted with one or more R.

77. The compound of any one of claim 1 to 72, or a pharmaceutically acceptable salt thereof, wherein R¹⁰ is selected from:

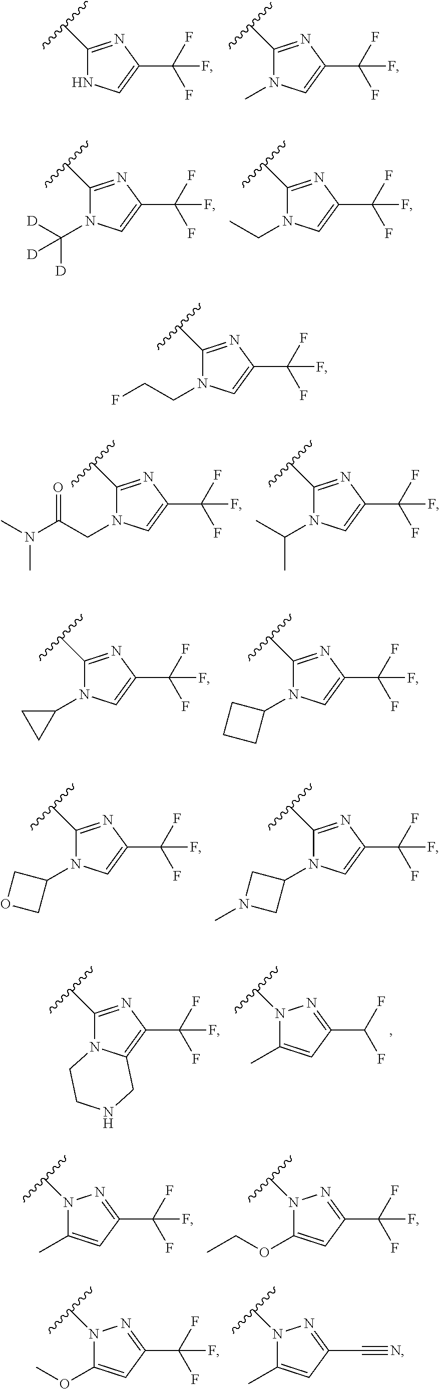

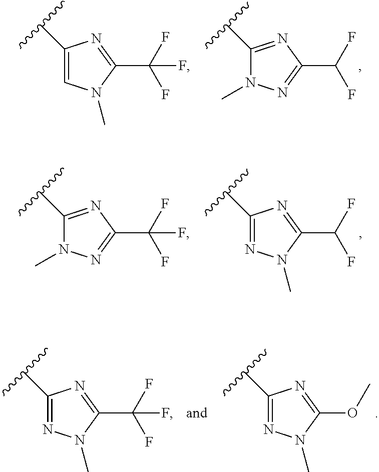

78. A compound of Table 1 or Table 2 or a pharmaceutically acceptable salt thereof.

79. A pharmaceutical composition comprising a compound of any one of claim 1 to 78, or a pharmaceutically acceptable salt thereof, and a pharmaceutically acceptable carrier or excipient.

80. A method of modulating ubiquitin specific protease 1 (USP1) in a subject, the method comprising administering to the subject a compound of any one of claim 1 to 78, or a pharmaceutically acceptable salt thereof, or a pharmaceutical composition of claim 79.

81. A method of inhibiting ubiquitin specific protease 1 (USP1) in a subject, the method comprising administering to the subject a compound of any one of claim 1 to 78, or a pharmaceutically acceptable salt thereof, or a pharmaceutical composition of claim 79.

82. A method of inhibiting or reducing DNA repair activity modulated by ubiquitin specific protease 1 (USP1) in a subject, the method comprising administering to the subject in need thereof an effective amount of a compound of any one of claim 1 to 78, or a pharmaceutically acceptable salt thereof, or a pharmaceutical composition of claim 79.

83. A method of treating a disease or disorder associated with ubiquitin specific protease 1 (USP1) in a subject, the method comprising administering to the subject a compound of any one of claim 1 to 78, or a pharmaceutically acceptable salt thereof, or a pharmaceutical composition of claim 79.

84. A method of treating a disease or disorder associated with modulation of ubiquitin specific protease 1 (USP1) in a subject, the method comprising administering to the subject a compound of any one of claim 1 to 78, or a pharmaceutically acceptable salt thereof, or a pharmaceutical composition of claim 79.

85. The method of claim 83 or 84, wherein the disease or disorder is cancer.

86. A method of treating cancer in a subject, the method comprising administering to the subject in need thereof an effective amount of a compound of any one of claim 1 to 78, or a pharmaceutically acceptable salt thereof, or a pharmaceutical composition of claim 79.

87. The method of claim 85 or 86, wherein the cancer is selected from the group consisting of lung cancer, non-small cell lung cancer (NSCLC), colon cancer, bladder cancer, osteosarcoma, ovarian cancer, skin cancer, and breast cancer.

88. The method of claim 85 or 86, wherein the cancer is ovarian cancer.

89. The method of claim 85 or 86, wherein the cancer is a breast cancer.

90. The method of claim 89, wherein the cancer is an ovarian cancer or breast cancer.

91. The method of any one of claim 85 to 90, wherein the cancer comprises cancer cells with elevated levels of RAD 18.

92. The method of any one of claim 85 to 91, wherein the cancer is a DNA damage repair pathway deficient cancer.

93. The method of any one of claim 85 to 92, wherein the cancer is a PARP inhibitor resistant or refractory cancer.

94. The method of any one of claim 85 to 93, wherein the cancer is a BRCA1 mutant cancer and/or a BRCA2 mutant cancer.

95. The method of claim 94, wherein the cancer is a BRAC1-deficient cancer.
